(19) United States
(12) Patent Application Publication
(10) Pub. No.: US 2026/0185095 A1
(43) Pub. Date: Jul. 2, 2026

(54) Compositions for Modulating Ataxin 2 Expression

(71) Applicant: Ionis Pharmaceuticals, Inc., Carlsbad, CA (US)

(72) Inventors: Susan M. Freier, San Diego, CA (US); Gene Hung, San Diego, CA (US); C. Frank Bennett, Carlsbad, CA (US)

(73) Assignee: Ionis Pharmaceuticals, Inc., Carlsbad, CA (US)

(21) Appl. No.: 19/545,773

(22) Filed: Feb. 20, 2026

Related U.S. Application Data
(63) Continuation of application No. 18/493,610, filed on Oct. 24, 2023, now Pat. No. 12,584,128.
Application No. 18/493,610 is a continuation of application No. 17/393,738, filed on Aug. 4, 2021, now Pat. No. 11,834,660.
Application No. 17/393,738 is a continuation of application No. 16/387,308, filed on Apr. 17, 2019, now Pat. No. 11,111,494.
Application No. 16/387,308 is a division of application No. 15/127,358, now Pat. No. 10,308,934.
(60) Provisional application No. 61/955,705, filed on Mar. 19, 2014.
(60) Provisional application No. 61/982,131, filed on Apr. 21, 2014.

(51) Int. Cl. C12N 15/113

(52) U.S. Cl. CPC C12N 15/113; C12N 2310/11; C12N 2310/315; C12N 2310/321; C12N 2310/3231; C12N 2310/3341; C12N 2310/341; C12N 2310/345; C12N 2310/346

(57) ABSTRACT

Disclosed herein are antisense compounds and methods for decreasing Ataxin 2 mRNA and protein expression. Such methods, compounds, and compositions are useful to treat, prevent, or ameliorate Ataxin 2 associated diseases, disorders, and conditions. Such Ataxin 2 associated diseases include spinocerebellar ataxia type 2 (SCA2), amyotropic sclerosis (ALS), and parkinsonism.

### SEQUENCE LISTING

The present application is being filed along with a Sequence Listing in electronic format. The Sequence Listing is provided as a file entitled BIOL0239SEQ.xml created Jun. 23, 2023, which is 299,836 bytes in size. The information in the electronic format of the sequence listing is incorporated herein by reference in its entirety.

### FIELD

Provided are compositions and methods for reducing expression of Ataxin 2 (ATXN2) mRNA and protein in an animal. Such methods are useful to treat, prevent, or ameliorate neurodegenerative diseases, including spinocerebellar ataxia type 2 (SCA2), amyotrophic lateral sclerosis (ALS), and parkinsonism by inhibiting expression of Ataxin 2 by inhibiting expression of Ataxin 2 in an animal.

### BACKGROUND

Spinocerebellar ataxia type 2 (SCA2) is an autosomal dominant neurodegenerative disease characterized by progressive functional and cell loss of neurons in the cerebellum, brain stem and spinal cord. The cause of SCA2 is CAG expansion in the ATXN2 gene resulting in polyglutamine (polyQ) expansion in the ataxin-2 protein. Patients with SCA2 are characterized by progressive cerebellar ataxia, slow saccadic eye movements and other neurologic features such as neuropathy (Pulst, S. M. (ed.), Genetics of Movement Disorders. Elsevier, Inc., Amsterdam, 2003, pp. 19-34.). Moderate CAG expansion in the ATXN2 gene is also associated with parkinsonism or amyotrophic lateral sclerosis (ALS) indistinguishable from the idiopathic forms of these diseases (Kim et al., Arch. Neurol., 2007, 64: 1510-1518; Ross et al., Hum. Mol. Genet., 2011, 20: 3207-3212; Corrado et al., Hum. Genet., 2011, 130: 575-580; Elden et al., Nature, 2010, 466: 1069-1075; Van Damme et al., Neurology, 2011, 76: 2066-2072).

The pathogenic functions of polyQ disease proteins that occur with polyQ expansion may be attributed to the gain of toxicity associated with the development of intranuclear inclusion bodies or with soluble toxic oligomers (Lajoie et al., PLoS One, 2011, 5: e15245). While SCA2 patient brains are characterized by loss of Purkinje cells, SCA2 Purkinje cells lack inclusion bodies indicating polyQ-expanded ataxin-2 may cause toxicity that is unrelated to inclusion body formation (Huynh et al., Ann. Neurol., 1999, 45: 232-241). Functions gained in polyQ-expanded ataxin-2 may include anomalous accumulation in Golgi bodies (Huynh et al., Hum. Mol. Genet., 2003, 12: 1485-1496), gain-of-normal functions (Duvick et al., Neuron, 2010, 67: 929-935) and sequestering of transcription factors (TFs) and glyceraldehyde-3-phosphate dehydrogenase like for other polyQ proteins (Yamanaka et al., Methods Mol. Biol., 2010: 648, 215-229; Koshy et al., Hum. Mol. Genet., 1996, 5: 1311-1318; Burke et al., Nat. Med., 1996, 2: 347-350). Some normal functions of ataxin-2 have been characterized. Ataxin-2 is present in stress granules and P-bodies suggesting functions in sequestering mRNAs and protein translation regulation during stress (Nonhoff et al., Mol. Biol. Cell, 2007, 18: 1385-1396). Ataxin-2 overexpression interfered with the P-body assembly, while underexpression interfered with stress granule assembly (Nonhoff et al., Mol. Biol. Cell, 2007, 18: 1385-1396). Interactions with polyA-binding protein 1, the RNA splicing factor A2BP1/Fox1 and polyribosomes further support roles for ataxin-2 in RNA metabolism (Shibata et al., Hum. Mol. Genet., 2000, 9: 1303-1313; Ciosk et al., Development, 2004, 131: 4831-4841; Satterfield et al., Hum. Mol. Genet., 2006, 15: 2523-2532). Ataxin-2 is a regulator of EGF receptor internalization and signaling by the way of its interactions with SRC kinase and the endocytic protein CIN85 (Nonis et al., Cell Signal., 2008, 20: 1725-1739). Ataxin-2 also interacts with the ALS-related protein TDP-43 in an RNA-dependent manner and familial and sporadic ALS associates with the occurrence of long normal CAG repeat expansion ATXN2 (Elden et al., Nature, 2010, 466: 1069-1075; Van Damme et al., Neurology, 2011, 76: 2066-2072).

Currently there is a lack of acceptable options for treating such neurodegenerative diseases. It is therefore an object herein to provide methods for the treatment of such diseases.

### SUMMARY

Provided herein are methods, compounds, and compositions for modulating expression of Ataxin 2 (ATXN2) mRNA and protein. In certain embodiments, compounds useful for modulating expression of Ataxin 2 mRNA and protein are antisense compounds. In certain embodiments, the antisense compounds are modified oligonucleotides.

In certain embodiments, modulation can occur in a cell or tissue. In certain embodiments, the cell or tissue is in an animal. In certain embodiments, the animal is a human. In certain embodiments, Ataxin 2 mRNA levels are reduced. In certain embodiments, Ataxin 2 protein levels are reduced. Such reduction can occur in a time-dependent manner or in a dose-dependent manner.

Also provided are methods, compounds, and compositions useful for preventing, treating, and ameliorating diseases, disorders, and conditions. In certain embodiments, such Ataxin 2 related diseases, disorders, and conditions are neurodegenerative diseases. In certain embodiments, such neurodegenerative diseases, disorders, and conditions include spinocerebellar ataxia type 2 (SCA2), amyotrophic lateral sclerosis (ALS), and parkinsonism.

Such diseases, disorders, and conditions can have one or more risk factors, causes, or outcomes in common. Certain risk factors and causes for development of neurodegenerative disorder include growing older, having a personal or family history, or genetic predisposition. Certain symptoms and outcomes associated with development of a neurodegenerative disorder include but are not limited to: ataxia, speech and swallowing difficulties, rigidity, tremors, ophthalmoplegia, saccadic slowing, peripheral neuropathy, atrophy, dystonia, chorea, and dementia.

In certain embodiments, methods of treatment include administering an Ataxin 2 antisense compound to an individual in need thereof. In certain embodiments, methods of treatment include administering an Ataxin 2 modified oligonucleotide to an individual in need thereof.

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Additionally, as used herein, the use of “and” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this disclosure, including, but not limited to, patents, patent applications, published patent applications, articles, books, treatises, and GENBANK Accession Numbers and associated sequence information obtainable through databases such as National Center for Biotechnology Information (NCBI) and other data referred to throughout in the disclosure herein are hereby expressly incorporated by reference for the portions of the document discussed herein, as well as in their entirety.

### Definitions

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis.

Unless otherwise indicated, the following terms have the following meanings:

“2′-O-methoxyethyl” (also 2′-MOE and 2′-OCH₂CH₂—OCH₃ and MOE) refers to an O-methoxyethyl modification of the 2′ position of a furanose ring. A 2′-O-methoxyethyl modified sugar is a modified sugar.

“2′-MOE nucleoside” (also 2′-O-methoxyethyl nucleoside) means a nucleoside comprising a 2′-MOE modified sugar moiety.

“2′-substituted nucleoside” means a nucleoside comprising a substituent at the 2′-position of the furanose ring other than H or OH. In certain embodiments, 2′ substituted nucleosides include nucleosides with bicyclic sugar modifications.

“5-methylcytosine” means a cytosine modified with a methyl group attached to the 5 position. A 5-methylcytosine is a modified nucleobase.

“About” means within ±7% of a value. For example, if it is stated, “the compounds affected at least about 70% inhibition of Ataxin 2”, it is implied that the Ataxin 2 levels are inhibited within a range of 63% and 77%.

“Administered concomitantly” refers to the co-administration of two pharmaceutical agents in any manner in which the pharmacological effects of both are manifest in the patient at the same time. Concomitant administration does not require that both pharmaceutical agents be administered in a single pharmaceutical composition, in the same dosage form, or by the same route of administration. The effects of both pharmaceutical agents need not manifest themselves at the same time. The effects need only be overlapping for a period of time and need not be coextensive.

“Administering” means providing a pharmaceutical agent to an animal, and includes, but is not limited to administering by a medical professional and self-administering.

“Amelioration” refers to a lessening, slowing, stopping, or reversing of at least one indicator of the seventy of a condition or disease. The severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antibody” refers to a molecule characterized by reacting specifically with an antigen in some way, where the antibody and the antigen are each defined in terms of the other. Antibody may refer to a complete antibody molecule or any fragment or region thereof, such as the heavy chain, the light chain, Fab region, and Fc region.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding. Examples of antisense compounds include single-stranded and double-stranded compounds, such as, antisense oligonucleotides, siRNAs, shRNAs, ssRNAs, and occupancy-based compounds.

“Antisense inhibition” means reduction of target nucleic acid levels in the presence of an antisense compound complementary to a target nucleic acid compared to target nucleic acid levels or in the absence of the antisense compound.

“Antisense mechanisms” are all those mechanisms involving hybridization of a compound with a target nucleic acid, wherein the outcome or effect of the hybridization is either target degradation or target occupancy with concomitant stalling of the cellular machinery involving, for example, transcription or splicing.

“Antisense oligonucleotide” means a single-stranded oligonucleotide having a nucleobase sequence that permits hybridization to a corresponding segment of a target nucleic acid.

“Ataxin 2” means the mammalian gene Ataxin 2 (ATXN2), including the human gene Ataxin 2 (ATXN2). Human Ataxin 2 has been mapped to human chromosome 12q24.1.

“Ataxin 2 associated disease” means any disease associated with any Ataxin 2 nucleic acid or expression product thereof. Such diseases may include a neurodegenerative disease. Such neurodegenerative diseases may include spinocerebellar ataxia type 2 (SCA2), amyotrophic lateral sclerosis (ALS), and parkinsonism.

“Ataxin 2 mRNA” means any messenger RNA expression product of a DNA sequence encoding Ataxin 2.

“Ataxin 2 nucleic acid” means any nucleic acid encoding Ataxin 2. For example, in certain embodiments, an Ataxin 2 nucleic acid includes a DNA sequence encoding Ataxin 2, an RNA sequence transcribed from DNA encoding Ataxin 2 (including genomic DNA comprising introns and exons), and an mRNA sequence encoding Ataxin 2. “Ataxin 2 mRNA” means an mRNA encoding an Ataxin 2 protein.

“Ataxin 2 protein” means the polypeptide expression product of an Ataxin 2 nucleic acid.

“Base complementarity” refers to the capacity for the precise base pairing of nucleobases of an antisense oligonucleotide with corresponding nucleobases in a target nucleic acid (i.e., hybridization), and is mediated by Watson-Crick, Hoogsteen or reversed Hoogsteen hydrogen binding between corresponding nucleobases.

“Bicyclic sugar” means a furanose ring modified by the bridging of two atoms. A bicyclic sugar is a modified sugar.

“Bicyclic nucleoside” (also BNA) means a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring.

“Cap structure” or “terminal cap moiety” means chemical modifications, which have been incorporated at either terminus of an antisense compound.

“cEt” or “constrained ethyl” means a bicyclic nucleoside having a sugar moiety comprising a bridge connecting the 4′-carbon and the 2′-carbon, wherein the bridge has the formula: 4′-CH(CH₃)—O-2′.

“Constrained ethyl nucleoside” (also cEt nucleoside) means a nucleoside comprising a bicyclic sugar moiety comprising a 4′-CH(CH₃)—O-2′ bridge.

“Chemically distinct region” refers to a region of an antisense compound that is in some way chemically different than another region of the same antisense compound. For example, a region having 2′-O-methoxyethyl nucleosides is chemically distinct from a region having nucleosides without 2′-O-methoxyethyl modifications.

“Chimeric antisense compound” means an antisense compound that has at least two chemically distinct regions, each position having a plurality of subunits.

“Co-administration” means administration of two or more pharmaceutical agents to an individual. The two or more pharmaceutical agents may be in a single pharmaceutical composition, or may be in separate pharmaceutical compositions. Each of the two or more pharmaceutical agents may be administered through the same or different routes of administration. Co-administration encompasses parallel or sequential administration.

“Complementarity” means the capacity for pairing between nucleobases of a first nucleic acid and a second nucleic acid.

“Comprise,” “comprises,” and “comprising” will be understood to imply the inclusion of a stated step or element or group of steps or elements but not the exclusion of any other step or element or group of steps or elements.

“Contiguous nucleobases” means nucleobases immediately adjacent to each other.

“Designing” or “designed to” refer to the process of designing an oligomeric compound that specifically hybridizes with a selected nucleic acid molecule.

“Diluent” means an ingredient in a composition that lacks pharmacological activity, but is pharmaceutically necessary or desirable. For example, in drugs that are injected, the diluent may be a liquid, e.g. saline solution.

“Dose” means a specified quantity of a pharmaceutical agent provided in a single administration, or in a specified time period. In certain embodiments, a dose may be administered in one, two, or more boluses, tablets, or injections. For example, in certain embodiments where subcutaneous administration is desired, the desired dose requires a volume not easily accommodated by a single injection, therefore, two or more injections may be used to achieve the desired dose. In certain embodiments, the pharmaceutical agent is administered by infusion over an extended period of time or continuously. Doses may be stated as the amount of pharmaceutical agent per hour, day, week, or month.

“Effective amount” in the context of modulating an activity or of treating or preventing a condition means the administration of that amount of pharmaceutical agent to a subject in need of such modulation, treatment, or prophylaxis, either in a single dose or as part of a series, that is effective for modulation of that effect, or for treatment or prophylaxis or improvement of that condition. The effective amount may vary among individuals depending on the health and physical condition of the individual to be treated, the taxonomic group of the individuals to be treated, the formulation of the composition, assessment of the individual's medical condition, and other relevant factors.

“Efficacy” means the ability to produce a desired effect.

“Expression” includes all the functions by which a gene's coded information is converted into structures present and operating in a cell. Such structures include, but are not limited to the products of transcription and translation.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region may be referred to as a “gap” and the external regions may be referred to as the “wings.”

“Gap-narrowed” means a chimeric antisense compound having a gap segment of 9 or fewer contiguous 2′-deoxyribonucleosides positioned between and immediately adjacent to 5′ and 3′ wing segments having from 1 to 6 nucleosides.

“Gap-widened” means a chimeric antisense compound having a gap segment of 12 or more contiguous 2′-deoxyribonucleosides positioned between and immediately adjacent to 5′ and 3′ wing segments having from 1 to 6 nucleosides.

“Hybridization” means the annealing of complementary nucleic acid molecules. In certain embodiments, complementary nucleic acid molecules include, but are not limited to, an antisense compound and a target nucleic acid. In certain embodiments, complementary nucleic acid molecules include, but are not limited to, an antisense oligonucleotide and a nucleic acid target.

“Identifying an animal having an Ataxin 2 associated disease” means identifying an animal having been diagnosed with an Ataxin 2 associated disease or predisposed to develop an Ataxin 2 associated disease. Individuals predisposed to develop an Ataxin 2 associated disease include those having one or more risk factors for developing an Ataxin 2 associated disease, including, growing older, having a personal or family history, or genetic predisposition of one or more Ataxin 2 associated diseases. Such identification may be accomplished by any method including evaluating an individual's medical history and standard clinical tests or assessments, such as genetic testing.

“Immediately adjacent” means there are no intervening elements between the immediately adjacent elements.

“Individual” means a human or non-human animal selected for treatment or therapy.

“Inhibiting Ataxin 2” means reducing the level or expression of an Ataxin 2 mRNA and/or protein. In certain embodiments, Ataxin 2 mRNA and/or protein levels are inhibited in the presence of an antisense compound targeting Ataxin 2, including an antisense oligonucleotide targeting Ataxin 2, as compared to expression of Ataxin 2 mRNA and/or protein levels in the absence of an Ataxin 2 antisense compound, such as an antisense oligonucleotide.

“Inhibiting the expression or activity” refers to a reduction or blockade of the expression or activity and does not necessarily indicate a total elimination of expression or activity.

“Internucleoside linkage” refers to the chemical bond between nucleosides.

“Linked nucleosides” means adjacent nucleosides linked together by an internucleoside linkage.

“Locked nucleic acid” or “LNA” or “LNA nucleosides” means nucleic acid monomers having a bridge connecting two carbon atoms between the 4′ and 2′position of the nucleoside sugar unit, thereby forming a bicyclic sugar. Examples of such bicyclic sugar include, but are not limited to A) α-L-Methyleneoxy (4′-CH₂—O-2′) LNA, (B) β-D-Methyleneoxy (4′-CH₂—O-2′) LNA, (C) Ethyleneoxy (4′-(CH₂)₂—O-2′) LNA, (D) Aminooxy (4′-CH₂—O—N(R)-2′) LNA and (E) Oxyamino (4′-CH₂—N(R)—O-2′) LNA, as depicted below.

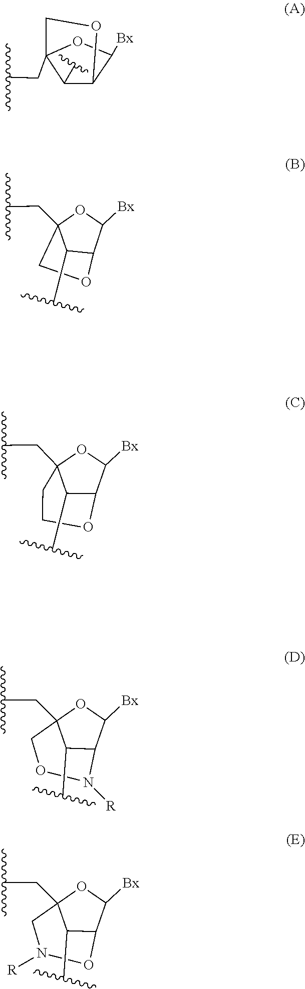

As used herein, LNA compounds include, but are not limited to, compounds having at least one bridge between the 4′ and the 2′ position of the sugar wherein each of the bridges independently comprises 1 or from 2 to 4 linked groups independently selected from —[C(R₁)(R₂)]ₙ—, —C(R₁)═C(R₂)—, —C(R₁)═N—, —C(═NR₁)—, —C(═O)—, —C(═S)—, —O—, —Si(R₁)₂—, —S(═O)ₓ— and —N(R₁)—; wherein: x is 0, 1, or 2; n is 1, 2, 3, or 4; each R₁ and R₂ is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, a heterocycle radical, a substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

Examples of 4′-2′ bridging groups encompassed within the definition of LNA include, but are not limited to one of formulae: —[C(R₁)(R₂)]ₙ—, —[C(R₁)(R₂)]ₙ—O—, —C(R₁R₂)—N(R₁)—O— or —C(R₁R₂)—O—N(R₁)—. Furthermore, other bridging groups encompassed with the definition of LNA are 4′-CH₂-2′, 4′-(CH₂)₂-2′, 4-(CH₂)₃-2′, 4′-CH₂—O-2′, 4′-(CH₂)₂—O-2′, 4′-CH₂—O—N(R₁)-2′ and 4′-CH₂—N(R₁)—O-2′-bridges, wherein each R₁ and R₂ is, independently, H, a protecting group or C₁-C₁₂ alkyl.

Also included within the definition of LNA according to the invention are LNAs in which the 2′-hydroxyl group of the ribosyl sugar ring is connected to the 4′ carbon atom of the sugar ring, thereby forming a methyleneoxy (4′-CH₂—O-2′) bridge to form the bicyclic sugar moiety. The bridge can also be a methylene (—CH₂—) group connecting the 2′ oxygen atom and the 4′ carbon atom, for which the term methyleneoxy (4′-CH₂—O-2′) LNA is used. Furthermore; in the case of the bicylic sugar moiety having an ethylene bridging group in this position, the term ethyleneoxy (4′-CH₂CH₂—O-2′) LNA is used. α-L-methyleneoxy (4′-CH₂—O-2′), an isomer of methyleneoxy (4′-CH₂—O-2′) LNA is also encompassed within the definition of LNA, as used herein.

“Mismatch” or “non-complementary nucleobase” refers to the case when a nucleobase of a first nucleic acid is not capable of pairing with the corresponding nucleobase of a second or target nucleic acid.

“Modified internucleoside linkage” refers to a substitution or any change from a naturally occurring internucleoside bond (i.e., a phosphodiester internucleoside bond).

“Modified nucleobase” means any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

A “modified nucleoside” means a nucleoside having, independently, a modified sugar moiety and/or modified nucleobase.

“Modified nucleotide” means a nucleotide having, independently, a modified sugar moiety, modified internucleoside linkage, and/or modified nucleobase.

“Modified oligonucleotide” means an oligonucleotide comprising at least one modified internucleoside linkage, modified sugar, and/or modified nucleobase.

“Modified sugar” means substitution and/or any change from a natural sugar moiety.

“Monomer” means a single unit of an oligomer. Monomers include, but are not limited to, nucleosides and nucleotides, whether naturally occurring or modified.

“Motif” means the pattern of unmodified and modified nucleosides in an antisense compound.

“Natural sugar moiety” means a sugar moiety found in DNA (2′-H) or RNA (2′-OH).

“Naturally occurring internucleoside linkage” means a 3′ to 5′ phosphodiester linkage.

“Non-complementary nucleobase” refers to a pair of nucleobases that do not form hydrogen bonds with one another or otherwise support hybridization.

“Nucleic acid” refers to molecules composed of monomeric nucleotides. A nucleic acid includes, but is not limited to, ribonucleic acids (RNA), deoxyribonucleic acids (DNA), single-stranded nucleic acids, double-stranded nucleic acids, small interfering ribonucleic acids (siRNA), and microRNAs (miRNA).

“Nucleobase” means a heterocyclic moiety capable of pairing with a base of another nucleic acid.

“Nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

“Nucleobase sequence” means the order of contiguous nucleobases independent of any sugar, linkage, and/or nucleobase modification.

“Nucleoside” means a nucleobase linked to a sugar.

“Nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo, or tricyclo sugar mimetics, e.g., non furanose sugar units. Nucleotide mimetic includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system. “Mimetic” refers to groups that are substituted for a sugar, a nucleobase, and/or internucleoside linkage. Generally, a mimetic is used in place of the sugar or sugar-internucleoside linkage combination, and the nucleobase is maintained for hybridization to a selected target.

“Nucleotide” means a nucleoside having a phosphate group covalently linked to the sugar portion of the nucleoside.

“Off-target effect” refers to an unwanted or deleterious biological effect associated with modulation of RNA or protein expression of a gene other than the intended target nucleic acid.

“Oligomeric compound” or “oligomer” means a polymer of linked monomeric subunits which is capable of hybridizing to at least a region of a nucleic acid molecule.

“Oligonucleotide” means a polymer of linked nucleosides each of which can be modified or unmodified, independent one from another.

“Parenteral administration” means administration through injection (e.g., bolus injection) or infusion. Parenteral administration includes subcutaneous administration, intravenous administration, intramuscular administration, intraarterial administration, intraperitoneal administration, or intracranial administration, e.g., intrathecal or intracerebroventricular administration.

“Peptide” means a molecule formed by linking at least two amino acids by amide bonds. Without limitation, as used herein, peptide refers to polypeptides and proteins.

“Pharmaceutical agent” means a substance that provides a therapeutic benefit when administered to an individual. For example, in certain embodiments, an antisense oligonucleotide targeted to Ataxin 2 is a pharmaceutical agent.

“Pharmaceutical composition” means a mixture of substances suitable for administering to a subject. For example, a pharmaceutical composition may comprise an antisense oligonucleotide and a sterile aqueous solution.

“Pharmaceutically acceptable derivative” encompasses pharmaceutically acceptable salts, conjugates, prodrugs or isomers of the compounds described herein.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevent” or “preventing” refers to delaying or forestalling the onset or development of a disease, disorder, or condition for a period of time from minutes to days, weeks to months, or indefinitely.

“Prodrug” means a therapeutic agent that is prepared in an inactive form that is converted to an active form (i.e., drug) within the body or cells thereof by the action of endogenous enzymes or other chemicals and/or conditions.

“Prophylactically effective amount” refers to an amount of a pharmaceutical agent that provides a prophylactic or preventative benefit to an animal.

“Region” is defined as a portion of the target nucleic acid having at least one identifiable structure, function, or characteristic.

“Ribonucleotide” means a nucleotide having a hydroxy at the 2′ position of the sugar portion of the nucleotide. Ribonucleotides may be modified with any of a variety of substituents.

“Salts” mean a physiologically and pharmaceutically acceptable salts of antisense compounds, i.e., salts that retain the desired biological activity of the parent oligonucleotide and do not impart undesired toxicological effects thereto.

“Segments” are defined as smaller or sub-portions of regions within a target nucleic acid.

“Shortened” or “truncated” versions of antisense oligonucleotides taught herein have one, two or more nucleosides deleted.

“Side effects” means physiological responses attributable to a treatment other than desired effects. In certain embodiments, side effects include, without limitation, injection site reactions, liver function test abnormalities, renal function abnormalities, liver toxicity, renal toxicity, central nervous system abnormalities, and myopathies.

“Single-stranded oligonucleotide” means an oligonucleotide which is not hybridized to a complementary strand.

“Sites,” as used herein, are defined as unique nucleobase positions within a target nucleic acid.

“Slows progression” means decrease in the development of the disease.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e., under physiological conditions in the case of in vivo assays and therapeutic treatments.

“Stringent hybridization conditions” or “stringent conditions” refer to conditions under which an oligomeric compound will hybridize to its target sequence, but to a minimal number of other sequences.

“Subject” means a human or non-human animal selected for treatment or therapy.

“Target” refers to a protein, the modulation of which is desired.

“Target gene” refers to a gene encoding a target.

“Targeting” or “targeted” means the process of design and selection of an antisense compound that will specifically hybridize to a target nucleic acid and induce a desired effect.

“Target nucleic acid,” “target RNA,” and “target RNA transcript” and “nucleic acid target” all mean a nucleic acid capable of being targeted by antisense compounds.

“Target region” means a portion of a target nucleic acid to which one or more antisense compounds is targeted.

“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.

“Therapeutically effective amount” means an amount of a pharmaceutical agent that provides a therapeutic benefit to an individual.

“Treat” or “treating” or “treatment” refers administering a composition to effect an alteration or improvement of the disease or condition.

“Unmodified nucleobases” mean the purine bases adenine (A) and guanine (G), and the pyrimidine bases thymine (T), cytosine (C) and uracil (U).

“Unmodified nucleotide” means a nucleotide composed of naturally occuring nucleobases, sugar moieties, and internucleoside linkages. In certain embodiments, an unmodified nucleotide is an RNA nucleotide (i.e. β-D-ribonucleosides) or a DNA nucleotide (i.e. β-D-deoxyribonucleoside).

“Wing segment” means a plurality of nucleosides modified to impart to an oligonucleotide properties such as enhanced inhibitory activity, increased binding affinity for a target nucleic acid, or resistance to degradation by in vivo nucleases.

### Certain Embodiments

Certain embodiments provide methods, compounds, and compositions for inhibiting Ataxin 2 mRNA and protein expression. Certain embodiments provide methods, compounds, and composition for decreasing Ataxin 2 mRNA and protein levels.

Certain embodiments provide antisense compounds targeted to an Ataxin 2 nucleic acid. In certain embodiments, the Ataxin 2 nucleic acid is the sequence set forth in GENBANK Accession No. NM_002973.3 (incorporated herein as SEQ ID NO: 1), the complement of GENBANK Accession No. NT_009775.17 truncated from nucleotides 2465000 to U.S. Pat. No. 2,616,000 (incorporated herein as SEQ ID NO: 2) and GENBANK Accession No. BX410018.2 (incorporated herein as SEQ ID NO: 3).

Certain embodiments provide methods for the treatment, prevention, or amelioration of diseases, disorders, and conditions associated with Ataxin 2 in an individual in need thereof. Also contemplated are methods for the preparation of a medicament for the treatment, prevention, or amelioration of a disease, disorder, or condition associated with Ataxin 2. Ataxin 2 associated diseases, disorders, and conditions include neurodegenerative diseases. In certain embodiments, Ataxin 2 associated diseases include spinocerebellar ataxia type 2 (SCA2), amyotrophic lateral sclerosis (ALS), and parkinsonism.

Certain embodiments provide compounds, comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or at least 20 consecutive nucleobases of any of the nucleobase sequences of SEQ ID NOs: 11-165.

In certain embodiments the nucleobase sequence of the modified oligonucleotide is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% complementary to SEQ ID NO: 1, SEQ ID NO: 2, or SEQ ID NO: 3.

In certain embodiments, the compound is a single-stranded modified oligonucleotide.

In certain embodiments, at least one internucleoside linkage of the modified oligonucleotide is a modified internucleoside linkage.

In certain embodiments, at least one modified internucleoside linkage is a phosphorothioate internucleoside linkage.

In certain embodiments, each modified internucleoside linkage is a phosphorothioate internucleoside linkage.

In certain embodiments, at least one internucleoside linkage is a phosphodiester internucleoside linkage.

In certain embodiments, at least one internucleoside linkage is a phosphorothioate linkage and at least one internucleoside linkage is a phosphodiester linkage.

In certain embodiments, at least one nucleoside comprises a modified nucleobase.

In certain embodiments, the modified nucleobase is a 5-methylcytosine.

In certain embodiments, at least one nucleoside of the modified oligonucleotide comprises a modified sugar.

In certain embodiments, at least one modified sugar is a bicyclic sugar.

In certain embodiments, the bicyclic sugar comprises a a chemical link between the 2′ and 4′ position of the sugar 4′-CH2-N(R)—O-2′ bridge wherein R is, independently, H, C1-C12 alkyl, or a protecting group.

In certain embodiments, the bicyclic sugar comprises a 4′-CH2-N(R)—O-2′ bridge wherein R is, independently, H, C1-C12 alkyl, or a protecting group.

In certain embodiments, at least one modified sugar comprises a 2′-O-methoxyethyl group.

In certain embodiments, the modified sugar comprises a 2′-O(CH₂)₂—OCH₃ group.

In certain embodiments, the modified oligonucleotide comprises:
a gap segment consisting of 10 linked deoxynucleosides;
a 5′ wing segment consisting of 5 linked nucleosides; and
a 3′ wing segment consisting of 5 linked nucleosides;
wherein the gap segment is positioned between the 5′ wing segment and the 3′ wing segment and wherein each nucleoside of each wing segment comprises a modified sugar.

In certain embodiments, the modified oligonucleotide consists of 20 linked nucleosides.

Certain embodiments provide compositions comprising any compound described herein or salt thereof and at least one of a pharmaceutically acceptable carrier or diluent.

Certain embodiments provide methods comprising administering to an animal any compound or composition described herein.

In certain embodiments, the animal is a human.

In certain embodiments, administering the compound prevents, treats, ameliorates, or slows progression of an Ataxin 2 associated disease, disorder or condition.

In certain embodiments, the Ataxin 2 disease, disorder or condition spinocerebellar ataxia type 2 (SCA2), amyotrophic lateral sclerosis (ALS), and parkinsonism.

Certain embodiments provide use of any of the compounds or compositions of described herein for the manufacture of a medicament for treating a neurodegenerative disorder.

### Antisense Compounds

Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound may be “antisense” to a target nucleic acid, meaning that is is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to an Ataxin 2 nucleic acid is 12 to 30 subunits in length. In certain embodiments, an antisense compound targeted to an Ataxin 2 nucleic acid is 12 to 25 subunits in length. In certain embodiments, an antisense compound targeted to an Ataxin 2 nucleic acid is 12 to 22 subunits in length. In certain embodiments, an antisense compound targeted to an Ataxin 2 nucleic acid is 14 to 20 subunits in length. In certain embodiments, an antisense compound targeted to an Ataxin 2 nucleic acid is 15 to 25 subunits in length. In certain embodiments, an antisense compound targeted to an Ataxin 2 nucleic acid is 18 to 22 subunits in length. In certain embodiments, an antisense compound targeted to an Ataxin 2 nucleic acid is 19 to 21 subunits in length. In certain embodiments, the antisense compound is 8 to 80, 12 to 50, 13 to 30, 13 to 50, 14 to 30, 14 to 50, 15 to 30, 15 to 50, 16 to 30, 16 to 50, 17 to 30, 17 to 50, 18 to 30, 18 to 50, 19 to 30, 19 to 50, or 20 to 30 linked subunits in length.

In certain embodiments, an antisense compound targeted to an Ataxin 2 nucleic acid is 12 subunits in length. In certain embodiments, an antisense compound targeted to an Ataxin 2 nucleic acid is 13 subunits in length. In certain embodiments, an antisense compound targeted to an Ataxin 2 nucleic acid is 14 subunits in length. In certain embodiments, an antisense compound targeted to an Ataxin 2 nucleic acid is 15 subunits in length. In certain embodiments, an antisense compound targeted to an Ataxin 2 nucleic acid is 16 subunits in length. In certain embodiments, an antisense compound targeted to an Ataxin 2 nucleic acid is 17 subunits in length. In certain embodiments, an antisense compound targeted to an Ataxin 2 nucleic acid is 18 subunits in length. In certain embodiments, an antisense compound targeted to an Ataxin 2 nucleic acid is 19 subunits in length. In certain embodiments, an antisense compound targeted to an Ataxin 2 nucleic acid is 20 subunits in length. In certain embodiments, an antisense compound targeted to an Ataxin 2 nucleic acid is 21 subunits in length. In certain embodiments, an antisense compound targeted to an Ataxin 2 nucleic acid is 22 subunits in length. In certain embodiments, an antisense compound targeted to an Ataxin 2 nucleic acid is 23 subunits in length. In certain embodiments, an antisense compound targeted to an Ataxin 2 nucleic acid is 24 subunits in length. In certain embodiments, an antisense compound targeted to an Ataxin 2 nucleic acid is 25 subunits in length. In certain embodiments, an antisense compound targeted to an Ataxin 2 nucleic acid is 26 subunits in length. In certain embodiments, an antisense compound targeted to an Ataxin 2 nucleic acid is 27 subunits in length. In certain embodiments, an antisense compound targeted to an Ataxin 2 nucleic acid is 28 subunits in length. In certain embodiments, an antisense compound targeted to an Ataxin 2 nucleic acid is 29 subunits in length. In certain embodiments, an antisense compound targeted to an Ataxin 2 nucleic acid is 30 subunits in length. In certain embodiments, the antisense compound targeted to an Ataxin 2 nucleic acid is 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In certain embodiments the antisense compound is an antisense oligonucleotide, and the linked subunits are nucleosides.

In certain embodiments antisense oligonucleotides targeted to an Ataxin 2 nucleic acid may be shortened or truncated. For example, a single subunit may be deleted from the 5′ end (5′ truncation), or alternatively from the 3′ end (3′ truncation). A shortened or truncated antisense compound targeted to an Ataxin 2 nucleic acid may have two subunits deleted from the 5′ end, or alternatively may have two subunits deleted from the 3′ end, of the antisense compound. Alternatively, the deleted nucleosides may be dispersed throughout the antisense compound, for example, in an antisense compound having one nucleoside deleted from the 5′ end and one nucleoside deleted from the 3′ end.

When a single additional subunit is present in a lengthened antisense compound, the additional subunit may be located at the 5′ or 3′ end of the antisense compound. When two or more additional subunits are present, the added subunits may be adjacent to each other, for example, in an antisense compound having two subunits added to the 5′ end (5′ addition), or alternatively to the 3′ end (3′ addition), of the antisense compound. Alternatively, the added subunits may be dispersed throughout the antisense compound, for example, in an antisense compound having one subunit added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Antisense Compound Motifs

In certain embodiments, antisense compounds targeted to an Ataxin 2 nucleic acid have chemically modified subunits arranged in patterns, or motifs, to confer to the antisense compounds properties such as enhanced inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may optionally serve as a substrate for the cellular endonuclease RNase H, which cleaves the RNA strand of an RNA:DNA duplex.

Antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may in some embodiments include β-D-ribonucleosides, β-D-deoxyribonucleosides, 2′-modified nucleosides (such 2′-modified nucleosides may include 2′-MOE, and 2′-O—CH₃, among others), and bicyclic sugar modified nucleosides (such bicyclic sugar modified nucleosides may include those having a 4′-(CH₂)n-O-2′ bridge, where n=1 or n=2 and 4′-CH₂—O—CH₂-2′). In certain embodiments, wings may include several modified sugar moieties, including, for example 2′-MOE. In certain embodiments, wings may include several modified and unmodified sugar moieties. In certain embodiments, wings may include various combinations of 2′-MOE nucleosides and 2′-deoxynucleosides.

Each distinct region may comprise uniform sugar moieties, variant, or alternating sugar moieties. The wing-gap-wing motif is frequently described as “X-Y-Z”, where “X” represents the length of the 5′ wing, “Y” represents the length of the gap, and “Z” represents the length of the 3′ wing. “X” and “Z” may comprise uniform, variant, or alternating sugar moieties. In certain embodiments, “X” and “Y” may include one or more 2′-deoxynucleosides. “Y” may comprise 2′-deoxynucleosides. As used herein, a gapmer described as “X-Y-Z” has a configuration such that the gap is positioned immediately adjacent to each of the 5′ wing and the 3′ wing. Thus, no intervening nucleotides exist between the 5′ wing and gap, or the gap and the 3′ wing. Any of the antisense compounds described herein can have a gapmer motif. In certain embodiments, “X” and “Z” are the same; in other embodiments they are different.

In certain embodiments, gapmers provided herein include, for example 20-mers having a motif of 5-10-5.

In certain embodiments, gapmers provided herein include, for example 19-mers having a motif of 5-9-5.

In certain embodiments, gapmers provided herein include, for example 18-mers having a motif of 5-8-5.

In certain embodiments, gapmers provided herein include, for example 18-mers having a motif of 4-8-6.

In certain embodiments, gapmers provided herein include, for example 18-mers having a motif of 6-8-4.

In certain embodiments, gapmers provided herein include, for example 18-mers having a motif of 5-7-6.

### Target Nucleic Acids, Target Regions and Nucleotide Sequences

Nucleotide sequences that encode Ataxin 2 include, without limitation, the following: GENBANK Accession No. NM_002973.3 (incorporated herein as SEQ ID NO: 1), the complement of GENBANK Accession No. NT_009775.17 truncated from nucleotides 2465000 to U.S. Pat. No. 2,616,000 (incorporated herein as SEQ ID NO: 2) and GENBANK Accession No. BX410018.2 (incorporated herein as SEQ ID NO: 3).

It is understood that the sequence set forth in each SEQ ID NO in the Examples contained herein is independent of any modification to a sugar moiety, an internucleoside linkage, or a nucleobase. As such, antisense compounds defined by a SEQ ID NO may comprise, independently, one or more modifications to a sugar moiety, an internucleoside linkage, or a nucleobase. Antisense compounds described by Isis Number (Isis No) indicate a combination of nucleobase sequence and motif.

In certain embodiments, a target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region. The structurally defined regions for Ataxin 2 can be obtained by accession number from sequence databases such as NCBI and such information is incorporated herein by reference. In certain embodiments, a target region may encompass the sequence from a 5′ target site of one target segment within the target region to a 3′ target site of another target segment within the same target region.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region may contain one or more target segments. Multiple target segments within a target region may be overlapping. Alternatively, they may be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceeding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

Suitable target segments may be found within a 5′ UTR, a coding region, a 3′ UTR, an intron, an exon, or an exon/intron junction. Target segments containing a start codon or a stop codon are also suitable target segments. A suitable target segment may specifically exclude a certain structurally defined region such as the start codon or stop codon.

The determination of suitable target segments may include a comparison of the sequence of a target nucleic acid to other sequences throughout the genome. For example, the BLAST algorithm may be used to identify regions of similarity amongst different nucleic acids. This comparison can prevent the selection of antisense compound sequences that may hybridize in a non-specific manner to sequences other than a selected target nucleic acid (i.e., non-target or off-target sequences).

There may be variation in activity (e.g., as defined by percent reduction of target nucleic acid levels) of the antisense compounds within an active target region. In certain embodiments, reductions in Ataxin 2 mRNA levels are indicative of inhibition of Ataxin 2 expression. Reductions in levels of an Ataxin 2 protein are also indicative of inhibition of target mRNA expression. Phenotypic changes are indicative of inhibition of Ataxin 2 expression. Improvement in neurological function is indicative of inhibition of Ataxin 2 expression. Improved motor function and memory are indicative of inhibition of Ataxin 2 expression.

### Hybridization

In some embodiments, hybridization occurs between an antisense compound disclosed herein and an Ataxin 2 nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense compounds provided herein are specifically hybridizable with an Ataxin 2 nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as an Ataxin 2 nucleic acid).

Non-complementary nucleobases between an antisense compound and an Ataxin 2 nucleic acid may be tolerated provided that the antisense compound remains able to specifically hybridize to a target nucleic acid. Moreover, an antisense compound may hybridize over one or more segments of an Ataxin 2 nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to an Ataxin 2 nucleic acid, a target region, target segment, or specified portion thereof. Percent complementarity of an antisense compound with a target nucleic acid can be determined using routine methods.

For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e., 100% complementary) to a target nucleic acid, or specified portion thereof. For example, an antisense compound may be fully complementary to an Ataxin 2 nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e., linked) or non-contiguous. In one embodiment, a non-complementary nucleobase is located in the wing segment of a gapmer antisense oligonucleotide.

In certain embodiments, antisense compounds that are, or are up to 11, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as an Ataxin 2 nucleic acid, or specified portion thereof.

In certain embodiments, antisense compounds that are, or are up to 11, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as an Ataxin 2 nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 9 nucleobase portion of a target segment. In certain embodiments, the antisense compounds are complementary to at least a 10 nucleobase portion of a target segment. In certain embodiments, the antisense compounds, are complementary to at least an 11 nucleobase portion of a target segment. In certain embodiments, the antisense compounds, are complementary to at least a 12 nucleobase portion of a target segment. In certain embodiments, the antisense compounds, are complementary to at least a 13 nucleobase portion of a target segment. In certain embodiments, the antisense compounds, are complementary to at least a 14 nucleobase portion of a target segment. In certain embodiments, the antisense compounds, are complementary to at least a 15 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

### Identity

The antisense compounds provided herein may also have a defined percent identity to a particular nucleotide sequence, SEQ ID NO, or compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, 75%, 80%, 85%, 90%, 95%, 96%, 97%, 98%, 99% or 100% identical to one or more of the antisense compounds or SEQ ID NOs, or a portion thereof, disclosed herein.

In certain embodiments, a portion of the antisense compound is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

In certain embodiments, a portion of the antisense oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occuring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to an Ataxin 2 nucleic acid comprise one or more modified internucleoside linkages. In certain embodiments, the modified internucleoside linkages are interspersed throughout the antisense compound. In certain embodiments, the modified internucleoside linkages are phosphorothioate linkages. In certain embodiments, each internucleoside linkage of an antisense compound is a phosphorothioate internucleoside linkage.

### Modified Sugar Moieties

Antisense compounds can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or S), 4-S, 2′-F, 2′-OCH₃, 2′-OCH₂CH₃, 2′-OCH₂CH₂F and 2′-O(CH₂)₂OCH₃ substituent groups. The substituent at the 2′ position can also be selected from allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(R)—(CH₂)₂—N(Rₘ)(Rₙ), where each Rₗ, Rₘ and Rₙ is, independently, H or substituted or unsubstituted C₁-C₁₀ alkyl.

As used herein, “bicyclic nucleosides” refer to modified nucleosides comprising a bicyclic sugar moiety. Examples of bicyclic nucleosides include without limitation nucleosides comprising a bridge between the 4′ and the 2′ ribosyl ring atoms. In certain embodiments, antisense compounds provided herein include one or more bicyclic nucleosides comprising a 4′ to 2′ bridge. Examples of such 4′ to 2′ bridged bicyclic nucleosides, include but are not limited to one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see published International Application WO/2009/006478, published Jan. 8, 2009); 4′-CH₂—N(OCH₃)-2′ (and analogs thereof see published International Application WO/2008/150729, published Dec. 11, 2008); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Chattopadhyaya et al., J. Org. Chem., 2009, 74, 118-134); and 4′-CH₂—C—(═CH₂)-2′ (and analogs thereof see published International Application WO 2008/154401, published on Dec. 8, 2008).

Further reports related to bicyclic nucleosides can also be found in published literature (see for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invest. Drugs, 2001, 2, 558-561; Braasch et al., Chem. Biol., 2001, 8, 1-7; and Orum et al., Curr. Opinion Mol. Ther., 2001, 3, 239-243; U.S. Pat. Nos. 6,268,490; 6,525,191; 6,670,461; 6,770,748; 6,794,499; 7,034,133; 7,053,207; 7,399,845; 7,547,684; and 7,696,345; U.S. Patent Publication No. US2008-0039618; US2009-0012281; U.S. Patent Serial Nos. 60/989,574; 61/026,995; 61/026,998; 61/056,564; 61/086,231; 61/097,787; and 61/099,844; Published PCT International applications WO 1994/014226; WO 2004/106356; WO 2005/021570; WO 2007/134181; WO 2008/150729; WO 2008/154401; and WO 2009/006478. Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226). In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═O)—, —C(═NRₐ)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is —[C(Rₐ)(R_{b})]ₙ—, —[C(Rₐ)(R_{b})]ₙ—O—, —C(RₐR_{b})—N(R)—O— or —C(RₐR_{b})—O—N(R)—. In certain embodiments, the bridge is 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R)-2′ and 4′-CH₂—N(R)—O-2′- wherein each R is, independently, H, a protecting group or C₁-C₁₂ alkyl.

In certain embodiments, bicyclic nucleosides are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, (A) α-L-methyleneoxy (4′-CH₂—O-2′) BNA, (B) β-D-methyleneoxy (4′-CH₂—O-2′) BNA, (C) ethyleneoxy (4′-(CH₂)₂—O-2′) BNA, (D) aminooxy (4′-CH₂—O—N(R)-2′) BNA, (E) oxyamino (4′-CH₂—N(R)—O-2′) BNA, and (F) methyl(methyleneoxy) (4′-CH(CH₃)—O-2′) BNA, (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, and (J) propylene carbocyclic (4′-(CH₂)₃-2′) BNA as depicted below.

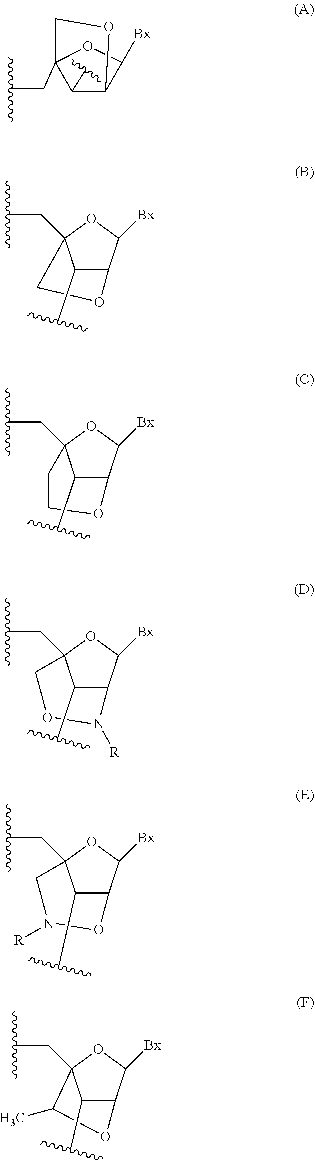

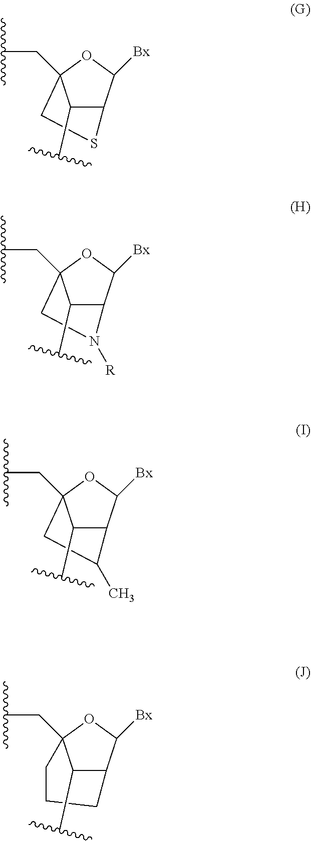

wherein Bx is the base moiety and R is independently H, a protecting group or C₁-C₁₂ alkyl.

In certain embodiments, bicyclic nucleosides are provided having Formula I:

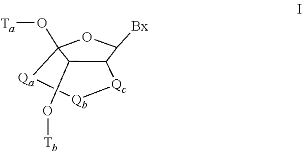

wherein:
   Bx is a heterocyclic base moiety;
   Qₐ-Q_{b}-Q_{c}- is —CH₂—N(R_{c})—CH₂—, —C(═O)—N(R_{c})—CH₂—, —CH₂—O—N(R_{c})—, —CH₂—N(R_{c})—O— or —N(R_{c})—O—CH₂;
   R_{c} is C₁-C₁₂ alkyl or an amino protecting group; and
   Tₐ and T_{b} are each, independently H, a hydroxyl protecting group, a conjugate group, a reactive phosphorus group, a phosphorus moiety or a covalent attachment to a support medium.

In certain embodiments, bicyclic nucleosides are provided having Formula II:

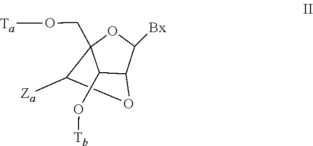

wherein:
   Bx is a heterocyclic base moiety;
   Tₐ and T_{b} are each, independently H, a hydroxyl protecting group, a conjugate group, a reactive phosphorus group, a phosphorus moiety or a covalent attachment to a support medium;
   Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol or substituted thio.

In one embodiment, each of the substituted groups is, independently, mono or poly substituted with substituent groups independently selected from halogen, oxo, hydroxyl, OJ_{c}, NJ_{c}J_{d}, SJ_{c}, N₃, OC(═X)J_{c}, and NJₑC(═X)NJ_{c}J_{d}, wherein each J_{c}, J_{d} and Jₑ is, independently, H, C₁-C₆ alkyl, or substituted C₁-C₆ alkyl and X is O or NJ_{c}.

In certain embodiments, bicyclic nucleosides are provided having Formula III:

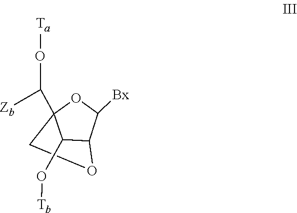

wherein:
   Bx is a heterocyclic base moiety;
   Tₐ and T_{b} are each, independently H, a hydroxyl protecting group, a conjugate group, a reactive phosphorus group, a phosphorus moiety or a covalent attachment to a support medium;
   Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl (C(═O)—).

In certain embodiments, bicyclic nucleosides are provided having Formula IV:

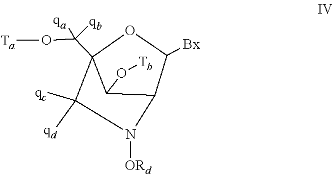

wherein:
   Bx is a heterocyclic base moiety;
   Tₐ and T_{b} are each, independently H, a hydroxyl protecting group, a conjugate group, a reactive phosphorus group, a phosphorus moiety or a covalent attachment to a support medium;
   R_{d} is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;
   each qₐ, q_{b}, q_{c} and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

In certain embodiments, bicyclic nucleosides are provided having Formula V:

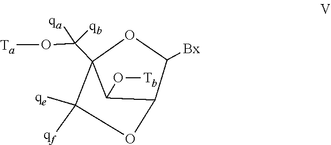

wherein:
   Bx is a heterocyclic base moiety;
   Tₐ and T_{b} are each, independently H, a hydroxyl protecting group, a conjugate group, a reactive phosphorus group, a phosphorus moiety or a covalent attachment to a support medium;
   qₐ, q_{b}, qₑ and q_{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;
   or qₑ and q_{f} together are ═C(q_{g})(qₕ);
   q_{g} and qₕ are each, independently, H, halogen, C₁-C₁₂ alkyl or substituted C₁-C₁₂ alkyl.

The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) BNA and 2′-thio-BNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-BNA, a novel comformationally restricted high-affinity oligonucleotide analog has been described in the art (Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported.

In certain embodiments, bicyclic nucleosides are provided having Formula VI:

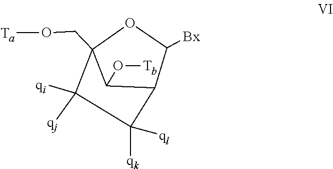

wherein:
   Bx is a heterocyclic base moiety;
   Tₐ and T_{b} are each, independently H, a hydroxyl protecting group, a conjugate group, a reactive phosphorus group, a phosphorus moiety or a covalent attachment to a support medium;
   each qᵢ, qₗ, qₖ and qₗ is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ; and
   qᵢ and qⱼ or qₗ and qₖ together are ═C(q_{g})(qₕ), wherein q_{g} and qₕ are each, independently, H, halogen, C₁-C₁₂ alkyl or substituted C₁-C₁₂ alkyl.

One carbocyclic bicyclic nucleoside having a 4′-(CH₂)₃-2′ bridge and the alkenyl analog bridge 4′-CH═CH—CH₂-2′ have been described (Freier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc., 2007, 129(26), 8362-8379).

As used herein, “4′-2′ bicyclic nucleoside” or “4′ to 2′ bicyclic nucleoside” refers to a bicyclic nucleoside comprising a furanose ring comprising a bridge connecting two carbon atoms of the furanose ring connects the 2′ carbon atom and the 4′ carbon atom of the sugar ring.

As used herein, “monocylic nucleosides” refer to nucleosides comprising modified sugar moieties that are not bicyclic sugar moieties. In certain embodiments, the sugar moiety, or sugar moiety analogue, of a nucleoside may be modified or substituted at any position.

As used herein, “2′-modified sugar” means a furanosyl sugar modified at the 2′ position. In certain embodiments, such modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH₂)ₙO]ₘCH₃, O(CH₂)ₙNH₂, O(CH₂)ₙCH₃, O(CH₂)ₙF, O(CH₂)ₙONH₂, OCH₂C(═O)N(H)CH₃, and O(CH₂)ₙON[(CH₂)ₙCH₃]₂, where n and m are from 1 to about 10. Other 2′-substituent groups can also be selected from: C₁-C₁₂ alkyl, substituted alkyl, alkenyl, alkynyl, alkaryl, aralkyl, O-alkaryl or O-aralkyl, SH, SCH₃, OCN, Cl, Br, CN, F, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an antisense compound, and other substituents having similar properties. In certain embodiments, modifed nucleosides comprise a 2′-MOE side chain (Baker et al., J. Biol. Chem., 1997, 272, 11944-12000). Such 2′-MOE substitution have been described as having improved binding affinity compared to unmodified nucleosides and to other modified nucleosides, such as 2′-O-methyl, O-propyl, and O-aminopropyl. Oligonucleotides having the 2′-MOE substituent also have been shown to be antisense inhibitors of gene expression with promising features for in vivo use (Martin, Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

As used herein, a “modified tetrahydropyran nucleoside” or “modified THP nucleoside” means a nucleoside having a six-membered tetrahydropyran “sugar” substituted in for the pentofuranosyl residue in normal nucleosides (a sugar surrogate). Modified THP nucleosides include, but are not limited to, what is referred to in the art as hexitol nucleic acid (HNA), anitol nucleic acid (ANA), manitol nucleic acid (MNA) (see Leumann, Bioorg. Med. Chem., 2002, 10, 841-854), fluoro HNA (F-HNA) or those compounds having Formula VII:

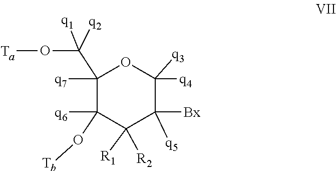

wherein independently for each of said at least one tetrahydropyran nucleoside analog of Formula VII:
   Bx is a heterocyclic base moiety;
   Tₐ and T_{b} are each, independently, an internucleoside linking group linking the tetrahydropyran nucleoside analog to the antisense compound or one of Tₐ and T_{b} is an internucleoside linking group linking the tetrahydropyran nucleoside analog to the antisense compound and the other of Tₐ and T_{b} is H, a hydroxyl protecting group, a linked conjugate group or a 5′ or 3′-terminal group;
   q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl; and each of R₁ and R₂ is selected from hydrogen, hydroxyl, halogen, substituted or unsubstituted alkoxy, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl.

In certain embodiments, the modified THP nucleosides of Formula VII are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, THP nucleosides of Formula VII are provided wherein one of R₁ and R₂ is fluoro. In certain embodiments, R₁ is fluoro and R₂ is H; R₁ is methoxy and R₂ is H, and R₁ is H and R₂ is methoxyethoxy.

As used herein, “2′-modified” or “2′-substituted” refers to a nucleoside comprising a sugar comprising a substituent at the 2′ position other than H or OH. 2′-modified nucleosides, include, but are not limited to, bicyclic nucleosides wherein the bridge connecting two carbon atoms of the sugar ring connects the 2′ carbon and another carbon of the sugar ring; and nucleosides with non-bridging 2′substituents, such as allyl, amino, azido, thio, O-allyl, O—C₁-C₁₀ alkyl, —OCF₃, O—(CH₂)₂O—CH₃, 2′-O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl. 2′-modifed nucleosides may further comprise other modifications, for example at other positions of the sugar and/or at the nucleobase.

As used herein, “2′-F” refers to a nucleoside comprising a sugar comprising a fluoro group at the 2′ position.

As used herein, “2′-OMe” or “2′-OCH₃” or “2′-O-methyl” each refers to a nucleoside comprising a sugar comprising an —OCH₃ group at the 2′ position of the sugar ring.

As used herein, “MOE” or “2′-MOE” or “2′-OCH₂CH₂OCH₃” or “2′-O-methoxyethyl” each refers to a nucleoside comprising a sugar comprising a —OCH₂CH₂OCH₃ group at the 2′ position of the sugar ring.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

Many other bicyclo and tricyclo sugar surrogate ring systems are also known in the art that can be used to modify nucleosides for incorporation into antisense compounds (see for example review article: Leumann, Bioorg. Med. Chem., 2002, 10, 841-854).

Such ring systems can undergo various additional substitutions to enhance activity.

Methods for the preparations of modified sugars are well known to those skilled in the art.

In nucleotides having modified sugar moieties, the nucleobase moieties (natural, modified or a combination thereof) are maintained for hybridization with an appropriate nucleic acid target.

In certain embodiments, antisense compounds comprise one or more nucleosides having modified sugar moieties. In certain embodiments, the modified sugar moiety is 2′-MOE. In certain embodiments, the 2′-MOE modified nucleosides are arranged in a gapmer motif. In certain embodiments, the modified sugar moiety is a bicyclic nucleoside having a (4′-CH(CH₃)—O-2′) bridging group. In certain embodiments, the (4′-CH(CH₃)—O-2′) modified nucleosides are arranged throughout the wings of a gapmer motif.

### Compositions and Methods for Formulating Pharmaceutical Compositions

Antisense oligonucleotides may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

An antisense compound targeted to an Ataxin 2 nucleic acid can be utilized in pharmaceutical compositions by combining the antisense compound with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense compound targeted to an Ataxin 2 nucleic acid and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

### Conjugated Antisense Compounds

Antisense compounds may be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

### Cell Culture and Antisense Compounds Treatment

The effects of antisense compounds on the level, activity or expression of Ataxin 2 nucleic acids can be tested in vitro in a variety of cell types. Cell types used for such analyses are available from commercial vendors (e.g. American Type Culture Collection, Manassas, VA; Zen-Bio, Inc., Research Triangle Park, NC; Clonetics Corporation, Walkersville, MD) and are cultured according to the vendor's instructions using commercially available reagents (e.g. Invitrogen Life Technologies, Carlsbad, CA). Illustrative cell types include, but are not limited to, HepG2 cells, Hep3B cells, and primary hepatocytes.

### In Vitro Testing of Antisense Oligonucleotides

Described herein are methods for treatment of cells with antisense oligonucleotides, which can be modified appropriately for treatment with other antisense compounds.

Cells may be treated with antisense oligonucleotides when the cells reach approximately 60-80% confluency in culture.

One reagent commonly used to introduce antisense oligonucleotides into cultured cells includes the cationic lipid transfection reagent LIPOFECTIN (Invitrogen, Carlsbad, CA). Antisense oligonucleotides may be mixed with LIPOFECTIN in OPTI-MEM 1 (Invitrogen, Carlsbad, CA) to achieve the desired final concentration of antisense oligonucleotide and a LIPOFECTIN concentration that may range from 2 to 12 μg/mL per 100 nM antisense oligonucleotide.

Another reagent used to introduce antisense oligonucleotides into cultured cells includes LIPOFECTAMINE (Invitrogen, Carlsbad, CA). Antisense oligonucleotide is mixed with LIPOFECTAMINE in OPTI-MEM 1 reduced serum medium (Invitrogen, Carlsbad, CA) to achieve the desired concentration of antisense oligonucleotide and a LIPOFECTAMINE concentration that may range from 2 to 12 μg/mL per 100 nM antisense oligonucleotide.

Another technique used to introduce antisense oligonucleotides into cultured cells includes electroporation.

Cells are treated with antisense oligonucleotides by routine methods. Cells may be harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTAMINE. Antisense oligonucleotides are used at higher concentrations ranging from 625 to 20,000 nM when transfected using electroporation.

### RNA Isolation

RNA analysis can be performed on total cellular RNA or poly(A)+mRNA. Methods of RNA isolation are well known in the art. RNA is prepared using methods well known in the art, for example, using the TRIZOL Reagent (Invitrogen, Carlsbad, CA) according to the manufacturer's recommended protocols.

### Analysis of Inhibition of Target Levels or Expression

Inhibition of levels or expression of an Ataxin 2 nucleic acid can be assayed in a variety of ways known in the art. For example, target nucleic acid levels can be quantitated by, e.g., Northern blot analysis, competitive polymerase chain reaction (PCR), or quantitative real-time PCR. RNA analysis can be performed on total cellular RNA or poly(A)+mRNA. Methods of RNA isolation are well known in the art. Northern blot analysis is also routine in the art. Quantitative real-time PCR can be conveniently accomplished using the commercially available ABI PRISM 7600, 7700, or 7900 Sequence Detection System, available from PE-Applied Biosystems, Foster City, CA and used according to manufacturer's instructions.

### Quantitative Real-Time PCR Analysis of Target RNA Levels

Quantitation of target RNA levels may be accomplished by quantitative real-time PCR using the ABI PRISM 7600, 7700, or 7900 Sequence Detection System (PE-Applied Biosystems, Foster City, CA) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents may be obtained from Invitrogen (Carlsbad, CA). RT real-time-PCR reactions are carried out by methods well known to those skilled in the art.

Gene (or RNA) target quantities obtained by real time PCR are normalized using either the expression level of a gene whose expression is constant, such as cyclophilin A, or by quantifying total RNA using RIBOGREEN (Invitrogen, Inc. Carlsbad, CA). Cyclophilin A expression is quantified by real time PCR, by being run simultaneously with the target, multiplexing, or separately. Total RNA is quantified using RIBOGREEN RNA quantification reagent (Invetrogen, Inc. Eugene, OR). Methods of RNA quantification by RIBOGREEN are taught in Jones, L. J., et al, (Analytical Biochemistry, 1998, 265, 368-374). A CYTOFLUOR 4000 instrument (PE Applied Biosystems) is used to measure RIBOGREEN fluorescence.

Probes and primers are designed to hybridize to an Ataxin 2 nucleic acid. Methods for designing real-time PCR probes and primers are well known in the art, and may include the use of software such as PRIMER EXPRESS Software (Applied Biosystems, Foster City, CA).

### Analysis of Protein Levels

Antisense inhibition of Ataxin 2 nucleic acids can be assessed by measuring Ataxin 2 protein levels. Protein levels of Ataxin 2 can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, MI), or can be prepared via conventional monoclonal or polyclonal antibody generation methods well known in the art.

### In Vivo Testing of Antisense Compounds

Antisense compounds, for example, antisense oligonucleotides, are tested in animals to assess their ability to inhibit expression of Ataxin 2 and produce phenotypic changes, such as, improved motor function and cognition. In certain embodiments, motor function is measured by walking initiation analysis, rotarod, grip strength, pole climb, open field performance, balance beam, hindpaw footprint testing in the animal.

Testing may be performed in normal animals, or in experimental disease models. For administration to animals, antisense oligonucleotides are formulated in a pharmaceutically acceptable diluent, such as phosphate-buffered saline. Administration includes parenteral routes of administration, such as intraperitoneal, intravenous, and subcutaneous. Calculation of antisense oligonucleotide dosage and dosing frequency is within the abilities of those skilled in the art, and depends upon factors such as route of administration and animal body weight. Following a period of treatment with antisense oligonucleotides, RNA is isolated from CNS tissue or CSF and changes in Ataxin 2 nucleic acid expression are measured.

### Certain Indications

In certain embodiments, provided herein are methods, compounds, and compositions of treating an individual comprising administering one or more pharmaceutical compositions described herein. In certain embodiments, the individual has a neurodegenerative disease. In certain embodiments, the individual is at risk for developing a neurodegenerative disease, including, but not limited to, spinocerebellar ataxia type 2 (SCA2), amyotrophic lateral sclerosis (ALS), and parkinsonism. In certain embodiments, the individual has been identified as having an Ataxin 2 associated disease. In certain embodiments, provided herein are methods for prophylactically reducing Ataxin 2 expression in an individual. Certain embodiments include treating an individual in need thereof by administering to an individual a therapeutically effective amount of an antisense compound targeted to an Ataxin 2 nucleic acid.

In one embodiment, administration of a therapeutically effective amount of an antisense compound targeted to an Ataxin 2 nucleic acid is accompanied by monitoring of Ataxin 2 levels in an individual, to determine an individual's response to administration of the antisense compound. An individual's response to administration of the antisense compound may be used by a physician to determine the amount and duration of therapeutic intervention.

In certain embodiments, administration of an antisense compound targeted to an Ataxin 2 nucleic acid results in reduction of Ataxin 2 expression by at least 15, 20, 25, 30, 35, 40, 45, 50, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or a range defined by any two of these values. In certain embodiments, administration of an antisense compound targeted to an Ataxin 2 nucleic acid results in improved motor function in an animal. In certain embodiments, administration of an Ataxin 2 antisense compound improves motor function by at least 15, 20, 25, 30, 35, 40, 45, 50, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%, or a range defined by any two of these values.

In certain embodiments, pharmaceutical compositions comprising an antisense compound targeted to Ataxin 2 are used for the preparation of a medicament for treating a patient suffering or susceptible to a neurodegenerative disease including spinocerebellar ataxia type 2 (SCA2), amyotrophic lateral sclerosis (ALS), and parkinsonism.

### EXAMPLES

### Non-Limiting Disclosure and Incorporation by Reference

While certain compounds, compositions, and methods described herein have been described with specificity in accordance with certain embodiments, the following examples serve only to illustrate the compounds described herein and are not intended to limit the same. Each of the references recited in the present application is incorporated herein by reference in its entirety.

### Example 1: Antisense Inhibition of Human Ataxin 2 in HepG2 Cells by MOE Gapmers

Antisense oligonucleotides were designed targeting an ataxin 2 nucleic acid and were tested for their effects on ataxin 2 mRNA in vitro. The antisense oligonucleotides were tested in a series of experiments that had similar culture conditions. The results for each experiment are presented in separate tables shown below. Cultured HepG2 cells at a density of 20,000 cells per well were transfected using electroporation with 4,500 nM antisense oligonucleotide. After a treatment period of approximately 24 hours, RNA was isolated from the cells and ataxin 2 mRNA levels were measured by quantitative real-time PCR. Human primer probe set RTS3642 (forward sequence ACCAAAGAGTAGTTAATGGAGGTGTTC, designated herein as SEQ ID NO: 5; reverse sequence AGAAGGTGGGCGAGAGGAA, designated herein as SEQ ID NO: 6; probe sequence CTGGCCATCGCCTTGCCCA, designated herein as SEQ ID NO: 7) was used to measure mRNA levels. Ataxin 2 mRNA levels were adjusted according to total RNA content, as measured by RIBOGREEN®. Results are presented as percent inhibition of ataxin 2, relative to untreated control cells.

The chimeric antisense oligonucleotides in the Tables below were designed as 5-10-5 MOE gapmers. The gapmers are 20 nucleosides in length, wherein the central gap segment is comprised often 2′-deoxynucleosides and is flanked by wing segments on the 5′ direction and the 3′ direction comprising five nucleosides each. Each nucleoside in the 5′ wing segment and each nucleoside in the 3′ wing segment has a 2′-MOE modification. The internucleoside linkages throughout each gapmer are phosphorothioate linkages. All cytosine residues throughout each gapmer are 5-methylcytosines. “Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either the human ataxin 2 mRNA, designated herein as SEQ ID NO: 1 (GENBANK Accession No. NM_002973.3) or the human ataxin 2 genomic sequence, designated herein as SEQ ID NO: 2 (the complement of GENBANK Accession No. NT_009775.17 truncated from nucleotides 2465000 to 2616000). Some oligonucleotides do not target either SEQ ID NO: 1 or SEQ ID NO: 2, but instead target a variant gene sequence, SEQ ID NO: 3 (GENBANK Accession No. BX410018.2). ‘n/a’ indicates that the antisense oligonucleotide does not target that particular gene sequence with 10000 complementarity.

TABLE 1
Inhibition of ataxin 2 mRNA by 5-10-5 MOE gapmers targeting SEQ ID NO: 1 and 2

| | SEQ | SEQ | | | | | |
|---|---|---|---|---|---|---|---|
| | ID | ID | | | SEQ | SEQ | |
| | NO: | NO: | | | ID | ID | |
| | 1 | 1 | | | NO: 2 | NO: 2 | SEQ |
| ISIS | Start | Stop | | % | Start | Stop | ID |
| NO | Site | Site | Sequence | inhibition | Site | Site | NO |
| 564118 | 606 | 625 | CCGGCTCGCACGCCGGGCGG | 57 | 2596 | 2615 | 11 |
| 564119 | 612 | 631 | CATACACCGGCTCGCACGCC | 63 | 2602 | 2621 | 12 |
| 564120 | 637 | 656 | GGCTTCAGCGACATGGTGAG | 78 | 2627 | 2646 | 13 |
| 564121 | 880 | 899 | CGACCTCTGCCCAGGCCGGG | 67 | n/a | n/a | 14 |
| 564122 | 935 | 954 | TGCATAGATTCCATCAAAAG | 90 | 47454 | 47473 | 15 |
| 564123 | 959 | 978 | AAGTATATGAACCATCCTCA | 67 | 47478 | 47497 | 16 |
| 564124 | 997 | 1016 | TTCACTTGTACTTCACATTT | 85 | 48696 | 48715 | 17 |
| 564125 | 1084 | 1103 | TCTGTACTTTTCTCATGTGC | 88 | 49258 | 49277 | 18 |
| 564126 | 1090 | 1109 | CTGGATTCTGTACTTTTCTC | 89 | 49264 | 49283 | 19 |
| 564127 | 1123 | 1142 | CTCTCCATTATTTCTTCACG | 92 | 49297 | 49316 | 20 |
| 564128 | 1168 | 1187 | TCTTTAAACTGTACCACAAC | 86 | 49342 | 49361 | 21 |
| 564129 | 1210 | 1229 | GAGTCAGTAAAAGCATCTCT | 84 | n/a | n/a | 22 |
| 564130 | 1264 | 1283 | CAGGGCTCCAGGTCCTTCTC | 83 | 76401 | 76420 | 23 |
| 564131 | 1270 | 1289 | GCATCCCAGGGCTCCAGGTC | 86 | 76407 | 76426 | 24 |
| 564132 | 1363 | 1382 | TCTTCATTATATCGAAACAT | 84 | 80718 | 80737 | 25 |
| 564133 | 1477 | 1496 | GCTAACTGGTTTGCCCTTGC | 98 | 81637 | 81656 | 26 |
| 564134 | 1556 | 1575 | GTATTTTTCTTCCTCACTCC | 82 | 81716 | 81735 | 27 |
| 564135 | 1562 | 1581 | TGCTGTGTATTTTTCTTCCT | 89 | 81722 | 81741 | 28 |
| 564136 | 1748 | 1767 | GAAATCTGAAGTGTGAGAAG | 61 | 83359 | 83378 | 29 |
| 564137 | 1789 | 1808 | CCTCCATTAACTACTCTTTG | 90 | 83400 | 83419 | 30 |
| 564138 | 1795 | 1814 | GGAACACCTCCATTAACTAC | 66 | n/a | n/a | 31 |
| 564139 | 1807 | 1826 | GGCGATGGCCAGGGAACACC | 95 | 85303 | 85322 | 32 |
| 564140 | 1844 | 1863 | GTAGCGAGAAGGTGGGCGAG | 88 | 85340 | 85359 | 33 |
| 564141 | 1862 | 1881 | AGAGTTGGGACCTGACTGGT | 84 | 85358 | 85377 | 34 |
| 564142 | 1868 | 1887 | TGGAAGAGAGTTGGGACCTG | 84 | 85364 | 85383 | 35 |
| 564143 | 1963 | 1982 | GGAGCTGGAGAACCATGAGC | 91 | 85459 | 85478 | 36 |
| 564144 | 1969 | 1988 | GAGACAGGAGCTGGAGAACC | 86 | 85465 | 85484 | 37 |
| 564145 | 2101 | 2120 | TTGTGGGATACAAATTCTAG | 56 | 88211 | 88230 | 38 |
| 564146 | 2185 | 2204 | GGAACCCCACTGACCACTGA | 70 | n/a | n/a | 39 |
| 564147 | 2401 | 2420 | TCTTGAAGCCTGGAATCTTT | 61 | 91671 | 91690 | 40 |
| 564148 | 2560 | 2579 | AACCTAAAATCATTCTTAAA | 21 | n/a | n/a | 41 |
| 564149 | 2596 | 2615 | AGTTGATCCATAGATTCAGA | 74 | 112905 | 112924 | 42 |
| 564150 | 2704 | 2723 | CTGGTACAGTTGCTGCTGCT | 91 | 113013 | 113032 | 43 |
| 564151 | 2710 | 2729 | CTGCCACTGGTACAGTTGCT | 85 | 113019 | 113038 | 44 |
| 564152 | 2899 | 2918 | TTTGCATTGGGATTCAATGT | 76 | 114859 | 114878 | 45 |
| 564153 | 2938 | 2957 | GAAGGCTTTGGCTGAGAGAA | 66 | n/a | n/a | 46 |
| 564154 | 2944 | 2963 | GTAGTAGAAGGCTTTGGCTG | 71 | n/a | n/a | 47 |
| 564155 | 2995 | 3014 | TGACCCACCATAGATGGGCT | 38 | 115850 | 115869 | 48 |
| 564156 | 3097 | 3116 | GGTATTGGGTATAAAGGTTG | 57 | n/a | n/a | 49 |
| 564157 | 3103 | 3122 | GTCATAGGTATTGGGTATAA | 76 | 116339 | 116358 | 50 |
| 564158 | 3331 | 3350 | GGATGCTGAGACTGATAATG | 54 | n/a | n/a | 51 |
| 564159 | 3337 | 3356 | ACATGAGGATGCTGAGACTG | 63 | n/a | n/a | 52 |
| 564160 | 3472 | 3491 | AATTTGGGACATGCATACAT | 23 | n/a | n/a | 53 |
| 564161 | 3490 | 3509 | GTCTCCTTGTTGTATGGTAA | 76 | 136963 | 136982 | 54 |
| 564162 | 3658 | 3677 | TGAACAGGACTGGGTGCAGG | 41 | 144433 | 144452 | 55 |
| 564163 | 3715 | 3734 | GACTGCTGCTGTGGACTGGC | 69 | 145447 | 145466 | 56 |
| 564164 | 3903 | 3922 | CTGACTGTACATGAGCCTGA | 50 | 147818 | 147837 | 57 |
| 564165 | 3909 | 3928 | CCATTCCTGACTGTACATGA | 69 | 147824 | 147843 | 58 |
| 564166 | 3927 | 3946 | CAGTTGGATGAGAAGGAACC | 58 | 147842 | 147861 | 59 |
| 564167 | 3933 | 3952 | CATGGGCAGTTGGATGAGAA | 29 | 147848 | 147867 | 60 |
| 564168 | 3971 | 3990 | ACCGCCGGGTGGCTGTGTCG | 40 | 147886 | 147905 | 61 |
| 564169 | 3993 | 4012 | TTTGAGCGAGGGCGGCCTGG | 19 | 147908 | 147927 | 62 |
| 564170 | 4005 | 4024 | GCTGTAGTGCACTTTGAGCG | 73 | 147920 | 147939 | 63 |
| 564171 | 4017 | 4036 | AGACTGGAATGGGCTGTAGT | 58 | 147932 | 147951 | 64 |
| 564172 | 4029 | 4048 | GCGCTGTTGTCGAGACTGGA | 74 | 147944 | 147963 | 65 |
| 564173 | 4035 | 4054 | GGAAATGCGCTGTTGTCGAG | 69 | 147950 | 147969 | 66 |
| 564174 | 4064 | 4083 | GGCTTGTACTGAAGGGTGCG | 23 | n/a | n/a | 67 |
| 564175 | 4070 | 4089 | GTGGTGGGCTTGTACTGAAG | 35 | n/a | n/a | 68 |
| 564176 | 4076 | 4095 | CTGTTGGTGGTGGGCTTGTA | 22 | 148827 | 148846 | 69 |
| 564177 | 4082 | 4101 | CAACTGCTGTTGGTGGTGGG | 39 | 148833 | 148852 | 70 |
| 564178 | 4088 | 4107 | GCCTTACAACTGCTGTTGGT | 62 | 148839 | 148858 | 71 |
| 564179 | 4106 | 4125 | TTCGGTTCCTCCAGGGCAGC | 72 | 148857 | 148876 | 72 |
| 564180 | 4166 | 4185 | TTCTAGTTTTCTGTGCTTCC | 72 | 148917 | 148936 | 73 |
| 564181 | 4367 | 4386 | AATAAATAACTTCCAGTTTC | 59 | 149118 | 149137 | 74 |
| 564182 | 4429 | 4448 | GAATCACTCTTGTTACTTCT | 78 | 149180 | 149199 | 75 |
| 564183 | 4435 | 4454 | CAGCAAGAATCACTCTTGTT | 85 | 149186 | 149205 | 76 |
| 564184 | 4551 | 4570 | TTTATAAATAATAATCCGTC | 4 | 149302 | 149321 | 77 |
| 564185 | 4593 | 4612 | AAGTTGAACCACTGTAGACA | 60 | 149344 | 149363 | 78 |
| 564186 | n/a | n/a | ATCGGCCACCACCCGCGCGC | 55 | 3683 | 3702 | 79 |
| 564187 | n/a | n/a | CAAAGGGTTAATTAGGATCT | 66 | 85057 | 85076 | 80 |
| 564188 | n/a | n/a | CCCAAAGGGTTAATTAGGAT | 94 | 85059 | 85078 | 81 |
| 564189 | n/a | n/a | AGGACAGTCATTTGATTTGT | 72 | 85166 | 85185 | 82 |
| 564190 | n/a | n/a | CTTTGAGGACAGTCATTTGA | 70 | 85171 | 85190 | 83 |
| 564191 | n/a | n/a | CTGACAGAACAAATGATATG | 17 | 85284 | 85303 | 84 |
| 564192 | n/a | n/a | TATTGGGTATAAAGGCTTGA | 31 | 116331 | 116350 | 85 |
| 564193 | n/a | n/a | GGTATTGGGTATAAAGGCTT | 78 | 116333 | 116352 | 86 |
| 564194 | n/a | n/a | CTCTTTTACGCATACAGGCA | 74 | 147789 | 147808 | 87 |
| 564195 | n/a | n/a | AGGAAGGCCAACTGAGTCCT | 70 | 148258 | 148277 | 88 |

TABLE 2
Inhibition of ataxin 2 mRNA by 5-10-5 MOE gapmers targeting SEQ ID NO: 1 and 2

| | SEQ | SEQ | | | | | |
|---|---|---|---|---|---|---|---|
| | ID | ID | | | SEQ | SEQ | |
| | NO: | NO: | | | ID | ID | |
| | 1 | 1 | | | NO: 2 | NO: 2 | SEQ |
| ISIS | Start | Stop | | % | Start | Stop | ID |
| NO | Site | Site | Sequence | inhibition | Site | Site | NO |
| 564158 | 3331 | 3350 | GGATGCTGAGACTGATAATG | 61 | n/a | n/a | 51 |
| 564196 | 70 | 89 | GGTCAGACGGAAGCAGAACG | 9 | 2060 | 2079 | 89 |
| 564197 | 218 | 237 | CCACCTGGCTGCGGCGAAGC | 12 | 2208 | 2227 | 90 |
| 564198 | 392 | 411 | GCCGTTGCCGTTGCTACCAA | 80 | 2382 | 2401 | 91 |
| 564199 | 616 | 635 | GGCCCATACACCGGCTCGCA | 79 | 2606 | 2625 | 92 |
| 564200 | 636 | 655 | GCTTCAGCGACATGGTGAGG | 81 | 2626 | 2645 | 93 |
| 564201 | 732 | 751 | GGACATTGGCAGCCGCGGGC | 83 | 2722 | 2741 | 94 |
| 564202 | 929 | 948 | GATTCCATCAAAAGAAATCG | 67 | n/a | n/a | 95 |
| 564203 | 969 | 988 | CAACTGATGTAAGTATATGA | 45 | 47488 | 47507 | 96 |
| 564204 | 1053 | 1072 | CCAAATCACACTTCGGACTG | 74 | n/a | n/a | 97 |
| 564205 | 1073 | 1092 | CTCATGTGCGGCATCAAGTA | 79 | 49247 | 49266 | 98 |
| 564206 | 1138 | 1157 | CATTTGAACAAAATACTCTC | 71 | 49312 | 49331 | 99 |
| 564207 | 1219 | 1238 | CTGATAGCAGAGTCAGTAAA | 72 | 76356 | 76375 | 100 |
| 564208 | 1521 | 1540 | GGGCCACTCGAGCTTTGTAC | 88 | 81681 | 81700 | 101 |
| 564209 | 1628 | 1647 | AGGAATATATTTATTTTCCC | 52 | 83239 | 83258 | 102 |
| 564210 | 1693 | 1712 | CCCATACGCGGTGAATTCTG | 91 | 83304 | 83323 | 103 |
| 564211 | 1713 | 1732 | TGGAGCCCGATCCAGGCTGG | 77 | 83324 | 83343 | 104 |
| 564212 | 1733 | 1752 | AGAAGTGGATCTTGATGGCA | 54 | 83344 | 83363 | 105 |
| 564213 | 1957 | 1976 | GGAGAACCATGAGCAGAGGG | 83 | 85453 | 85472 | 106 |
| 564214 | 2002 | 2021 | GGCCCTTCTGAAGACATGCG | 85 | n/a | n/a | 107 |
| 564215 | 2079 | 2098 | CACTGGATATGGAACCCCTC | 84 | 88189 | 88208 | 108 |
| 564216 | 2099 | 2118 | GTGGGATACAAATTCTAGGC | 94 | 88209 | 88228 | 109 |
| 564217 | 2177 | 2196 | ACTGACCACTGATGACCACG | 67 | 88287 | 88306 | 110 |
| 564218 | 2215 | 2234 | CTGGGTCTATGAGTTTTAGG | 67 | 91099 | 91118 | 111 |
| 564219 | 2291 | 2310 | TGGAATAATACCAGCTTGGG | 84 | 91175 | 91194 | 112 |
| 564220 | 2311 | 2330 | GGCATGGCAACAGCTTCAGT | 81 | 91195 | 91214 | 113 |
| 564221 | 2331 | 2350 | TAGGAGATGCAGCTGGAATA | 71 | 91215 | 91234 | 114 |
| 564222 | 2397 | 2416 | GAAGCCTGGAATCTTTAGCC | 69 | n/a | n/a | 115 |
| 564223 | 2426 | 2445 | CCCTGCAGGAGAGTTCTGCC | 75 | 91696 | 91715 | 116 |
| 564224 | 2582 | 2601 | TTCAGAAGTAGAACTTGGCT | 76 | 112891 | 112910 | 117 |
| 564225 | 2652 | 2671 | CAATTTTGTCTTTGATCAAA | 56 | 112961 | 112980 | 118 |
| 564226 | 2757 | 2776 | TGTTACTAAGTATTGAAGGG | 53 | 113066 | 113085 | 119 |
| 564227 | 2787 | 2806 | AAGTGACCTCAGGTCCCCTC | 83 | 113096 | 113115 | 120 |
| 564228 | 2883 | 2902 | ATGTTGATTTCCTAACTTGC | 53 | 114843 | 114862 | 121 |
| 564229 | 3019 | 3038 | GTATAAACTGGAGTTGGCTG | 75 | 115874 | 115893 | 122 |
| 564230 | 3039 | 3058 | GTGCAAAACAAACAGGCTGA | 79 | 115894 | 115913 | 123 |
| 564231 | 3059 | 3078 | GACTGGATACATCATATTTG | 18 | 115914 | 115933 | 124 |
| 564232 | 3082 | 3101 | GGTTGCACGCCTGGGCTCAC | 74 | n/a | n/a | 125 |
| 564233 | 3102 | 3121 | TCATAGGTATTGGGTATAAA | 50 | 116338 | 116357 | 126 |
| 564234 | 3122 | 3141 | TTGATTCACTGGCATGGGCG | 77 | 116358 | 116377 | 127 |
| 564235 | 3180 | 3199 | GATGATGCTGGTCTTGCCGC | 49 | 130944 | 130963 | 128 |
| 564236 | 3373 | 3392 | ATCATTCTAGCATTACCCTG | 61 | 131454 | 131473 | 129 |
| 564237 | 3408 | 3427 | ATACTAAACCAGGCTGGGCG | 71 | 131489 | 131508 | 130 |
| 564238 | 3464 | 3483 | ACATGCATACATCGCATGCG | 32 | n/a | n/a | 131 |
| 564239 | 3505 | 3524 | TAGAAAGAAGGGCTTGTCTC | 67 | 136978 | 136997 | 132 |
| 564240 | 3545 | 3564 | CGCATACTGCTGAGCAAGGG | 79 | 144320 | 144339 | 133 |
| 564241 | 3597 | 3616 | TAGCTGAAGGCTGAGGGTGT | 43 | 144372 | 144391 | 134 |
| 564242 | 3630 | 3649 | CACCATGTTGGCTTTGCTGC | 81 | 144405 | 144424 | 135 |
| 564243 | 3650 | 3669 | ACTGGGTGCAGGATGACTTC | 36 | 144425 | 144444 | 136 |
| 564244 | 3729 | 3748 | CGTGGTAAATGGCTGACTGC | 50 | 145461 | 145480 | 137 |
| 564245 | 3772 | 3791 | TTGGAGGCAGGTGTCATGGA | 36 | 145504 | 145523 | 138 |
| 564246 | 3938 | 3957 | TGGCGCATGGGCAGTTGGAT | 67 | 147853 | 147872 | 139 |
| 564247 | 3994 | 4013 | CTTTGAGCGAGGGCGGCCTG | 29 | 147909 | 147928 | 140 |
| 564248 | 4021 | 4040 | GTCGAGACTGGAATGGGCTG | 54 | 147936 | 147955 | 141 |
| 564249 | 4225 | 4244 | ATTCCTATTGGATGTTACAA | 65 | 148976 | 148995 | 142 |
| 564250 | 4252 | 4271 | ATCTTCCACTGCAAGTGAAC | 77 | 149003 | 149022 | 143 |
| 564251 | 4306 | 4325 | TATGGAATTATGGAATAGCC | 65 | 149057 | 149076 | 144 |
| 564252 | 4433 | 4452 | GCAAGAATCACTCTTGTTAC | 77 | 149184 | 149203 | 145 |
| 564253 | 4581 | 4600 | TGTAGACAGTGATCACCTCA | 77 | 149332 | 149351 | 146 |
| 564254 | n/a | n/a | GGCCAAGGCCCACTTGTCTC | 54 | 3485 | 3504 | 147 |
| 564255 | n/a | n/a | CACTGCGGCCTCGAACAGCA | 81 | 3709 | 3728 | 148 |
| 564263 | n/a | n/a | AAATTCCTCATTTTCTTTTC | 68 | 26924 | 26943 | 149 |
| | | | | | 27239 | 27258 | |
| 564264 | n/a | n/a | GTTATAGTAATCTGTAATCA | 71 | 36133 | 36152 | 150 |
| | | | | | 36239 | 36258 | |
| 564265 | n/a | n/a | AGGATTGTAAAATGATACAG | 47 | 65107 | 65126 | 151 |
| | | | | | 65148 | 65167 | |
| 564266 | n/a | n/a | GTAGGATTGTAAAATGATAC | 64 | 65109 | 65128 | 152 |
| | | | | | 65150 | 65169 | |
| 564267 | n/a | n/a | TTATATATGTAAATTATATC | 9 | 95228 | 95247 | 153 |
| | | | | | 95288 | 95307 | |
| 564268 | n/a | n/a | AACCACTGATTTATACACTT | 88 | 95260 | 95279 | 154 |
| | | | | | 95320 | 95339 | |
| 564269 | n/a | n/a | TTAAAAACCACTGATTTATA | 17 | 95265 | 95284 | 155 |
| | | | | | 95325 | 95344 | |
| 564270 | n/a | n/a | ATATAGCACTCTGCTGTATT | 83 | 99282 | 99301 | 156 |
| | | | | | 99340 | 99359 | |
| 564271 | n/a | n/a | TACCAAGCTTGTGGCTTGGG | 32 | 137342 | 137361 | 157 |
| | | | | | 137420 | 137439 | |
| 564272 | n/a | n/a | TTATACCAAGCTTGTGGCTT | 52 | 137345 | 137364 | 158 |
| | | | | | 137423 | 137442 | |

TABLE 3
Inhibition of ataxin 2 mRNA by 5-10-5 MOE gapmers
targeting SEQ ID NO: 3

| | SEQ | SEQ | | | |
|---|---|---|---|---|---|
| | ID | ID | | | |
| | NO: | NO: | | | |
| | 3 | 3 | | % | SEQ |
| ISIS | Start | Stop | | inhibi- | ID |
| No | Site | Site | Sequence | tion | NO |
| 564256 | 311 | 330 | CCTCGATGTTCCACAGGCGC | 83 | 159 |
| 564257 | 715 | 734 | GAGTTCACCTGCATCCAGGG | 81 | 160 |
| 564258 | 736 | 755 | TCCAGTTCCCTCATTGGCTG | 27 | 161 |
| 564259 | 771 | 790 | GGTTCCATCCATTAGATACG | 52 | 162 |
| 564260 | 791 | 810 | TTAAACGAAACATATCTTTG | 10 | 163 |
| 564261 | 815 | 834 | GCCCCTGCGCCATAATTTTT | 3 | 164 |
| 564262 | 835 | 854 | ATAAACTGCTTTCAACGGTG | 2 | 165 |

### Example 2: Dose-Dependent Antisense Inhibition of Human Ataxin 2 in HepG2 Cells by MOE Gapmers

Gapmers from Example 1 exhibiting significant in vitro inhibition of ataxin 2 mRNA were selected and tested at various doses in HepG2 cells. Cells were plated at a density of 20,000 cells per well and transfected using electroporation with 0.625 μM, 1.250 μM, 2.500 μM, 5.000 μM and 10.000 μM concentrations of antisense oligonucleotide, as specified in the Table below. After a treatment period of approximately 16 hours, RNA was isolated from the cells and ataxin 2 mRNA levels were measured by quantitative real-time PCR. Human primer probe set RTS3642 was used to measure mRNA levels. Ataxin 2 mRNA levels were adjusted according to total RNA content, as measured by RIBOGREEN®. Results are presented as percent inhibition of ataxin 2, relative to untreated control cells.

The half maximal inhibitory concentration (IC₅₀) of each oligonucleotide is also presented. Ataxin 2 mRNA levels were significantly reduced in a dose-dependent manner in antisense oligonucleotide treated cells.

TABLE 4
Dose response assay

| ISIS | 0.625 | 1.250 | 2.500 | 5.000 | 10.000 | IC₅₀ |
|---|---|---|---|---|---|---|
| No | μM | μM | μM | μM | μM | (μM) |
| 564133 | 89 | 95 | 98 | 98 | 97 | <0.6 |
| 564188 | 52 | 72 | 81 | 88 | 90 | <0.6 |
| 564127 | 42 | 62 | 65 | 85 | 91 | 0.8 |
| 564150 | 39 | 63 | 74 | 86 | 91 | 0.8 |
| 564143 | 37 | 60 | 76 | 84 | 94 | 0.9 |
| 564122 | 25 | 53 | 69 | 85 | 88 | 1.3 |
| 564126 | 23 | 48 | 61 | 78 | 89 | 1.7 |
| 564144 | 12 | 35 | 53 | 71 | 85 | 2.4 |
| 564135 | 22 | 35 | 53 | 73 | 86 | 2.1 |
| 564125 | 33 | 44 | 64 | 78 | 85 | 1.5 |
| 564129 | 31 | 42 | 54 | 71 | 77 | 1.9 |
| 564216 | 50 | 67 | 82 | 86 | 94 | <0.6 |
| 564210 | 33 | 48 | 72 | 80 | 94 | 1.3 |
| 564208 | 30 | 40 | 67 | 75 | 87 | 1.6 |
| 564268 | 35 | 52 | 69 | 81 | 85 | 1.2 |

### Example 3: Antisense Inhibition of Human Ataxin 2 in a SCA2 BAC Mouse Model

Gapmers from Example 1 exhibiting significant in vitro inhibition of ataxin 2 mRNA were selected and tested in vivo in a SCA2[Q22]-BAC mouse model. This mouse model was created in the Pulst laboratory (University of Utah, Salt Lake City), using mice of FVB/B6 hybrid background, for the study of spinocerebella ataxia type 2 (SCA2). These mice possess the entire 176 kb human ATXN2 gene region, including the 16 kb upstream sequence and the 2.5 kb downstream sequence.

### Treatment

Groups of 3 mice each were administered normal saline (0.9%) or antisense oligonucleotide via intracerebroventricular injections. Five to seven week old mice were individually infused with a mixture of oxygen and 3% isoflurane for 3-4 minutes to cause sedation. The hair on the scalp was then removed with a shearing tool. The mouse was placed in a stereotaxic instrument (Stoelting Just for Mouse). The scalp was cleaned, first with an iodine scrub, and then with 70% ethanol. An incision was made with a #10 scalpel blade from the region just posterior to the place between the eyes to the region 1.5 cm behind. The periosteum was removed with a sterile cotton swab. A Hamilton syringe with a 26-gauge needle was placed in the needle holder of the stereotaxic instrument and filled up to the 10 μL mark with either normal saline (0.9%) or antisense oligonucleotide (250 μg) in saline (0.9%) solution. The needle was positioned on the bregma on the skull, and then positioned 1 mm to the right and 0.46 mm posterior. The tip of the needle was then inserted just through the skull and then positioned 2.5 mm down into the right lateral ventricle. The plunger of the syringe was then depressed to deliver the desired volume of 5-7 μL. After a wait of 4 minutes to allow ventricular pressure to equalize, the needle was removed and the scalp was sutured. The incision was then treated with povidone solution and the mouse returned to its cage on its back for recovery. The mice were monitored daily.

### RNA Analysis

After 7 days, the mice were placed in isoflurane until they were no longer breathing. The brain was then extracted. Three portions of the brain were collected in coronal sections, including one 3 mm section for RNA analysis. RNA was isolated from 30 mg tissue using the RNeasy kit (Qiagen). cDNA was generated using the QuantiTect Reverse Transcription kit (Qiagen). Real-time PCR (qPCR) was conducted by the SYBR Green method with standard curves on the iCycler (Bio-Rad) in 96-well plates in quadruplicate. Reactions were of 20 μL, consisting of 15 ng cDNA, 2 μL of each primer (0.3 μM final), and 10 μL SYBR Green Master Mix (Bio-Rad). Cycling parameters included a 95° denaturation step for 10 seconds, incubation at the annealing temperature for 20 seconds, and a second incubation for 40 seconds at 72°. Each plate included a standard curve using cerebellar RNA prepared from multiple pGL2-5A3 transgenic mice. Single amplicons were verified by denaturation analysis and gel electrophoresis.

The results from the RNA analysis for mouse and human ataxin 2 are presented in the Table below. As indicated, some of the ISIS oligonucleotides decreased human ataxin 2 mRNA in the brains of the mice.

TABLE 5
Percent inhibition of ataxin 2 mRNA compared
to the saline (0.9%) control in SCA[Q22]-BAC mice

| ISIS | Human | Mouse |
|---|---|---|
| No | ataxin 2 | ataxin 2 |
| 564122 | 10 | 15 |
| 564127 | 46 | 65 |
| 564133 | 60 | 62 |
| 564150 | 21 | 53 |
| 564188 | 9 | 23 |
| 564216 | 21 | 55 |

### Example 4: Antisense Inhibition of Human Ataxin 2 in an ATXN2-Q127 Mouse Model

Gapmers from Example 1 exhibiting significant in vitro inhibition of ataxin 2 mRNA were selected and tested in vivo in an ATXN2-Q127 mouse model. This mouse model (Hansen, S. T. et al., Human. Molecular Genetics 2012. 1-13) expresses the full-length-mutant ATXN2^{Q127} complementary DNA under the regulation of the Purkinje cell protein-2 (Pcp2) promoter. This model shows an early-onset progressive motor impairment phenotype accompanied by the formation of diffuse cytoplasmic aggregates in cerebellar Purkinje cells.

### Treatment

Groups of 3 mice each were administered normal saline (0.9%) or antisense oligonucleotide via intracerebroventricular injections. Five to seven week old mice were individually infused with a mixture of oxygen and 3% isoflurane for 3-4 minutes to cause sedation. The hair on the scalp was then removed with a shearing tool. The mouse was placed in a stereotaxic instrument (Stoelting Just for Mouse). The scalp was cleaned, first with an iodine scrub, and then with 70% ethanol. An incision was made with a #10 scalpel blade from the region just posterior to the place between the eyes to the region 1.5 cm behind. The periosteum was removed with a sterile cotton swab. A Hamilton syringe with a 26-gauge needle was placed in the needle holder of the stereotaxic instrument and filled up to the 10 μL mark with either normal saline (0.9%) or antisense oligonucleotide (250 μg) in saline (0.9%) solution. The needle was positioned on the bregma on the skull, and then positioned 1 mm to the right and 0.46 mm posterior. The tip of the needle was then inserted just through the skull and then positioned 2.5 mm down into the right lateral ventricle. The plunger of the syringe was then depressed to deliver the desired volume of 5-7 μL. After a wait of 4 minutes to allow ventricular pressure to equalize, the needle was removed and the scalp was sutured. The incision was then treated with povidone solution and the mouse returned to its cage on its back for recovery. The mice were monitored daily.

### RNA Analysis

After 7 days, the mice were placed in isoflurane until they were no longer breathing. The brain was then extracted. Three portions of the brain were collected in coronal sections, including one 3 mm section for RNA analysis. RNA was isolated from 30 mg tissue using the RNeasy kit (Qiagen). cDNA was generated using the QuantiTect Reverse Transcription kit (Qiagen). Real-time PCR (qPCR) was conducted by the SYBR Green method with standard curves on the iCycler (Bio-Rad) in 96-well plates in quadruplicate. Reactions were of 20 μL, consisting of 15 ng cDNA, 2 μL of each primer (0.3 μM final), and 10 μL SYBR Green Master Mix (Bio-Rad). Cycling parameters included a 95° denaturation step for 10 seconds, incubation at the annealing temperature for 20 seconds, and a second incubation for 40 seconds at 72°. Each plate included a standard curve using cerebellar RNA prepared from multiple pGL2-5A3 transgenic mice. Single amplicons were verified by denaturation analysis and gel electrophoresis. All mRNA levels were normalized to the housekeeping gene, actin.

The results from the RNA analysis for mouse and human ataxin 2 are presented in the Table below. As indicated, some of the ISIS oligonucleotides decreased human ataxin 2 mRNA in the brains of the mice.

qPCR analysis of the marker for microgliosis, AIF/Iba1, to measure inflammation, was also performed. The results are presented in the Table below.

TABLE 6
Percent inhibition of ataxin 2 mRNA compared
to the saline (0.9%) control in ATXN2-Q127 mice

| ISIS | | |
|---|---|---|
| No | Human | Mouse |
| 564133 | 64 | 52 |
| 564127 | 62 | 49 |
| 564216 | 46 | 40 |
| 564210 | 39 | 48 |

TABLE 7
Percent Iba1 mRNA level increase compared to
the saline (0.9%) control in ATXN2-Q127 mice

| ISIS | |
|---|---|
| No | Iba1 |
| 564133 | 9 |
| 564127 | 49 |
| 564216 | 16 |
| 564210 | 96 |

### Example 4: Dose-Dependent Antisense Inhibition of Human Ataxin 2 in an ATXN2-Q127 Mouse Model

ISIS 564133 was tested in different doses in the ATXN2-Q127 mouse model.

### Treatment

Groups of 3 mice each were administered normal saline (0.9%) or ISIS 564133 via intracerebroventricular injections dosed at 50 μg, 100 μg, 200 μg, 250 μg, or 300 μg. The mice were administered in the same manner as described in the studies above and monitored daily.

### RNA Analysis

After 7 days, the mice were placed in isoflurane until they were no longer breathing. The brain was then extracted. Three portions of the brain were collected in coronal sections, including one 3 mm section for RNA analysis, as described above. All mRNA levels were normalized to the housekeeping gene, actin.

The results from the RNA analysis for mouse and human ataxin 2 are presented in the Table below.

TABLE 8
Percent inhibition of ataxin 2 mRNA compared
to the saline (0.9%) control in ATXN2-Q127 mice

| Dose | Human | Mouse |
|---|---|---|
| (μg) | ataxin 2 | ataxin 2 |
| 50 | 60 | 47 |
| 100 | 84 | 35 |
| 200 | 85 | 67 |
| 250 | 79 | 62 |
| 300 | 73 | 41 |

### Example 5: Time-Dependent Antisense Inhibition of Human Ataxin 2 in an ATXN2-Q127 Mouse Model

ISIS 564133 was administered and mRNA level reduction was tested in different time points in the ATXN2-Q127 mouse model.

### Treatment

Groups of 3 mice each were administered normal saline (0.9%) or ISIS 564133 via intracerebroventricular injections dosed at 200 μg. The mice were administered in the same manner as described in the studies above and monitored daily.

### RNA Analysis

After 9 days, 18 days, 27 days, and 84 days, groups of mice were placed in isoflurane until they were no longer breathing. The brain was then extracted. Three portions of the brain were collected in coronal sections, including one 3 mm section for RNA analysis, as described above. All mRNA levels were normalized to the housekeeping gene, actin.

The results from the RNA analysis for human ataxin 2 are presented in the Table below. Western analysis of the corresponding protein samples was performed and confirmed the qPCR results.

TABLE 9
Ataxin 2 mRNA levels in ATXN2-Q127 mice

| | ATXN2 expression |
|---|---|
| Time Point | relative to actin |
| saline (0.9%) | 8.4 |
| control | |
| 9 days | 2.9 |
| 18 days | 0.9 |
| 27 days | 1.4 |
| 84 days | 2.7 |

Immunohistochemical staining of cerebellar Purkinje cells on day 7 was performed using rabbit anti-oligonucleotide antibody generated in-house. The results demonstrated that ISIS oligonucleotide localized in cerebellar Purkinje cells of ATXN-Q127 mice.

### Example 6: Effect of Antisense Inhibition of Human Ataxin 2 in an ATXN2-Q127 Mouse Model

ISIS oligonucleotide was administered in the ATXN2-Q127 mouse model and wild-type mice. On day 3, motor performance was evaluated using the rotarod test.

Groups of ATXN2-Q127 mice were administered normal saline (0.9%) or ISIS 564133 at 50 μg, 100 μg, or 200 μg via intracerebroventricular injections in the same manner as described in the studies above. Groups of wild-type mice were administered normal saline (0.9%) or ISIS oligonucleotide at 200 μg via intracerebroventricular injections dosed in the same manner as described in the studies above. Groups of ATXN2-Q127 mice were administered normal saline (0.9%) or ISIS 546127 or ISIS 564216 at 200 μg via intracerebroventricular injections dosed in the same manner as described in the studies above. After 6 weeks, the mice were subjected to the rotarod test.

### Rotarod Assay

The accelerating rotarod assay was performed on the Rotamex rotarod. Rotarod testing was conducted over five days. On the first day, mice are acclimated to the technician by handling the mice. On the second day mice are introduced to the rotarod in a 4 minutes paradigm including 2 minutes at a constant speed of 10 RPM, then 2 minutes at a speed ranging from 10 to 30 RPM. Testing on days 3-5 were identical, where mice are placed on the rotarod at a speed of 0 RPM, then the rotarod was accelerated to 40 RPM over 6 minutes. This is done twice per day and a mean value of “latency to fall” per day was recorded, in seconds. Latency to fall is defined as the amount of time before the animal falls from the rotarod. It is recorded automatically, when the mouse no longer interrupts infrared beams directed above the rotarod. The time to first passive rotation (when mice stop walking and hold on and revolve with the rod) is also automatically recorded, and generally reflects the latency to fall time. The study consisted of three consecutive trials of 5 minutes each with a 20 minute rest period between trials. On days 3-5, the mice were allowed to rest for 1.5-2 hrs between the two replicate tests conducted on each of those days.

The results from the rotarod test are presented in the Table below. As shown in the Table below, treatment with ASO improves rotarod performance by up to about 20%.

TABLE 10
Rotarod performance test in ATXN2-Q127 mice

| Strain | Number of | | Latency to |
|---|---|---|---|
| of mice | mice | Treatment | fall (seconds) |
| WT | 10 | saline (0.9%) | 199 |
| | | control | |
| | 10 | ISIS 564133 | 189 |
| | | (200 μg) | |
| ATXN-Q127 | 8 | saline (0.9%) | 127 |
| | | control | |
| | 15 | ISIS 564133 | 149 |
| | | (50 μg) | |
| | 16 | ISIS 564133 | 141 |
| | | (100 μg) | |
| | 9 | ISIS 564133 | 100 |
| | | (200 μg) | |
| ATXN-Q127 | 15 | saline (0.9%) | 130 |
| | | control | |
| | 13 | ISIS 564127 | 150 |
| | | (200 μg) | |
| | 15 | ISIS 564216 | 156 |
| | | (200 μg) | |

### Example 7: Effect of Antisense Inhibition of Human Ataxin 2 in an ATXN2-Q127 Mouse Model

ISIS oligonucleotide was administered in the ATXN2-Q127 mouse model and wild-type mice. Cerebellar expression of ataxin 2, as well as several Purkinje cell (PC) genes, was assessed.

Groups of ATXN2-Q127 mice were administered normal saline (0.9%) or ISIS 564133 at 200 μg via intracerebroventricular injections dosed in the same manner as described in the studies above. Groups of wild-type mice were administered normal saline (0.9%) or ISIS 564133 at 200 μg via intracerebroventricular injections dosed in the same manner as described in the studies above. After 5 weeks, the mice were euthanized and cerebellar expression of various gene mRNA levels was assessed.

### RNA Analysis

Groups of mice were placed in isoflurane until they were no longer breathing. The brain was then extracted. Three portions of the brain were collected in coronal sections, including one 3 mm section for RNA analysis, as described above. All mRNA levels were normalized to the housekeeping gene, actin. RNA levels of human ataxin 2, murine ataxin 2, Pcp2, Calb1, Rgs8, and Fam107b were measured. Transcription changes in several of these PC-specific genes have been demonstrated to progressively decrease in models of SCA2 (Hansen, S. T. et al., Hum. Mol. Genet. 2013. 22: 271-283).

The results from the RNA analysis are presented in the Table below and demonstrate that treatment with ISIS oligonucleotides targeting ataxin 2 increased the expression levels of all the PC-specific genes compared to the transgenic control group.

TABLE 11
PC-specific mRNA levels in ATXN2-Q127 mice

| | WT | ATXN-Q127 | |
|---|---|---|---|
| | saline (0.9%) | saline (0.9%) | ISIS 564133 |
| | control | control | (200 μg) |
| human ataxin 2 | 0.21 | 3.57 | 1.31 |
| murine ataxin 2 | 0.79 | 0.84 | 0.6 |
| Pcp2 | 0.77 | 0.36 | 0.48 |
| Rgs8 | 1.45 | 0.25 | 0.35 |
| Calb1 | 1.14 | 0.5 | 0.71 |
| Fam107b | 1.41 | 0.7 | 0.9 |

### Example 8: Effect of Antisense Inhibition of Human Ataxin 2 in an ATXN2-Q127 Mouse Model

ISIS oligonucleotide was administered in the ATXN2-Q127 mouse model and wild-type mice. Motor performance was evaluated using the rotarod test.

Groups of ATXN2-Q127 mice (7.5 weeks of age) were administered normal saline (0.9%) or ISIS 546127 or ISIS 564216 at 200 μg via intracerebroventricular injections dosed in the same manner as described in the studies above. After 5 weeks and 9 weeks, the mice were subjected to the rotarod test.

### Rotarod Assay

The accelerating rotarod assay was performed on the Rotamex rotarod. Rotarod testing was conducted over five days. On the first day, mice are acclimated to the technician by handling the mice. On the second day mice are introduced to the rotarod in a 4 minutes paradigm including 2 minutes at a constant speed of 10 RPM, then 2 minutes at a speed ranging from 10 to 30 RPM. Testing on days 3-5 were identical, where mice are placed on the rotarod at a speed of 0 RPM, then the rotarod was accelerated to 40 RPM over 6 minutes. This is done twice per day and a mean value of “latency to fall” per day was recorded, in seconds. Latency to fall is defined as the amount of time before the animal falls from the rotarod. It is recorded automatically, when the mouse no longer interrupts infrared beams directed above the rotarod. The time to first passive rotation (when mice stop walking and hold on and revolve with the rod) is also automatically recorded, and generally reflects the latency to fall time. The study consisted of three consecutive trials of 5 minutes each with a 20 minute rest period between trials. On days 3-5, the mice were allowed to rest for 1.5-2 hrs between the two replicate tests conducted on each of those days.

The results from the rotarod test are presented in the Table below. As shown in the Table below, treatment with ASO improves rotarod performance by up to about 20% on week 5 and about 27% on week 9.

TABLE 12
Rotarod performance test in ATXN2-Q127 mice.
(mean latency to fall, in seconds)

| Weeks after | | ISIS | ISIS | Saline |
|---|---|---|---|---|
| injection | | 564127 | 564216 | control |
| Week 5 | DAY 3 | 137 | 145 | 123 |
| | DAY 4 | 140 | 141 | 119 |
| | DAY 5 | 155 | 154 | 131 |
| Week 9 | DAY 3 | 131 | 149 | 104 |
| | DAY 4 | 125 | 139 | 104 |
| | DAY 5 | 134 | 139 | 112 |

### Example 9: Effect of Antisense Inhibition of Human Ataxin 2 in an ATXN2-Q127 Mouse Model

ISIS oligonucleotide was administered in the ATXN2-Q127 mouse model. Motor performance was evaluated using the rotarod test.

Seven week old ATXN2-Q127 mice were subjected to the rotarod test, then divided into two groups of 30 mice each, such that average rotarod performance, average weights, and sex composition were equal across both groups. At 8 weeks of age, one group of mice received normal saline via intracerebroventricular (ICV) injection and one group received ISIS 564216 at 210 μg via ICV injection, dosed in the same manner as described in the studies above. Five weeks later (13 weeks of age), the mice were again subjected to the rotarod test. Six weeks post injection (14 weeks of age), the mice received a second ICV injection, identical to the injection received at 8 weeks of age. Five weeks later (19 weeks of age, 11 weeks after the first ICV injection), the mice were subjected to a third rotarod test.

### Rotarod Test

The accelerating rotarod test was performed on the Rotamex rotarod. Rotarod testing was conducted over five days. On the first day, mice were acclimated to the technician by being handled by the technician three times, 5 minutes each time. On the second day, mice were introduced to the rotarod three times, 10 minutes each time at a speed ranging from 0 to 10 RPM. On each of days 3-5, mice were placed on the rotarod at a speed of 0 RPM, then the rotarod was accelerated to 40 RPM over 6 minutes, and this was done for each mouse three times. The three total trials per day were used to calculate a mean value of “latency to fall” per day, in seconds. Latency to fall is defined as the amount of time before the animal falls from the rotarod. It was recorded automatically, when the mouse no longer interrupted infrared beams directed above the rotarod. The time to first passive rotation (when mice stop walking and hold on and revolve with the rod) is also automatically recorded, and generally reflects the latency to fall time.

The results from the rotarod test are presented as the average for each treatment group in the Table below. As shown in the Table below, treatment with ASO improved rotarod performance.

TABLE 13
Rotarod performance test in ATXN2-Q127 mice

| | Weeks after 1^{st} | Weeks after 2^{nd} | Testing | Latency |
|---|---|---|---|---|
| Treatment | injection | injection | day | to fall (s) |
| Saline | 5 | n/a | 3 | 218.5 |
| | | | 4 | 240.9 |
| | | | 5 | 236.5 |
| Isis No. | 5 | n/a | 3 | 240.6 |
| 564216 | | | 4 | 257.9 |
| | | | 5 | 259.6 |
| Saline | 11 | 5 | 3 | 216.2 |
| | | | 4 | 198.7 |
| | | | 5 | 212.1 |
| Isis No. | 11 | 5 | 3 | 194.4 |
| 564216 | | | 4 | 226.0 |
| | | | 5 | 242.8 |

What is claimed is:

1. A compound, comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or at least 20 consecutive nucleobases of any of the nucleobase sequences of SEQ ID NOs: 11-165.

2. The compound of claim 2, wherein the nucleobase sequence of the modified oligonucleotide is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% complementary to SEQ ID NO: 1, SEQ ID NO: 2, or SEQ ID NO: 3.

3. The compound of any preceding claim, consisting of a single-stranded modified oligonucleotide.

4. The compound of any preceding claim, wherein at least one internucleoside linkage is a modified internucleoside linkage.

5. The compound of claim 4, wherein at least one modified internucleoside linkage is a phosphorothioate internucleoside linkage.

6. The compound of claim 4, wherein each modified internucleoside linkage is a phosphorothioate internucleoside linkage.

7. The compound of any preceding claim, wherein at least one internucleoside linkage is a phosphodiester internucleoside linkage.

8. The compound of any preceding claim, wherein at least one internucleoside linkage is a phosphorothioate linkage and at least one internucleoside linkage is a phosphodiester linkage.

9. The compound of any preceding claim, wherein at least one nucleoside comprises a modified nucleobase.

10. The compound of claim 9, wherein the modified nucleobase is a 5-methylcytosine.

11. The compound of any preceding claim, wherein at least one nucleoside of the modified oligonucleotide comprises a modified sugar.

12. The compound of claim 11, wherein the at least one modified sugar is a bicyclic sugar.

13. The compound of claim 12, wherein the bicyclic sugar comprises a 4′-CH(R)—O-2′ bridge wherein R is, independently, H, C₁-C₁₂ alkyl, or a protecting group.

14. The compound of claim 13, wherein R is methyl.

15. The compound of claim 13, wherein R is H.

16. The compound of claim 11, wherein the at least one modified sugar comprises a 2′-O-methoxyethyl group.

17. The compound of any preceding claim, wherein the modified oligonucleotide comprises:
a gap segment consisting of 10 linked deoxynucleosides;
a 5′ wing segment consisting of 5 linked nucleosides; and
a 3′ wing segment consisting of 5 linked nucleosides;
wherein the gap segment is positioned between the 5′ wing segment and the 3′ wing segment and wherein each nucleoside of each wing segment comprises a modified sugar.

18. The compound of any preceding claim, wherein the modified oligonucleotide consists of 20 linked nucleosides.

19. A composition comprising the compound of any preceding claim or salt thereof and at least one of a pharmaceutically acceptable carrier or diluent.

20. A method comprising administering to an animal the compound or composition of any preceding claim.

21. The method of claim 20, wherein the animal is a human.

22. The method of claims 20 and 21, wherein the administering the compound prevents, treats, ameliorates, or slows progression of an Ataxin 2 associated disease, disorder or condition.

23. The method of claim 22, wherein the disease, disorder or condition is spinocerebellar ataxia type 2 (SCA2), amyotrophic lateral sclerosis (ALS), or parkinsonism.

24. Use of the compound or composition of any preceding claim for the manufacture of a medicament for treating a neurodegenerative disorder.


SEQUENCE LISTING (165 sequences)

SEQ ID NO: 1 - RNA, 4712 nt, Homo sapiens
  source 1..4712: mol_type = mRNA
acccccgagaaagcaacccagcgcgccgcccgctcctcacgtgtccctcccggccccggggccacctcacgttctgcttccgtctgacccctccgacttccggtaaagagtccctatccgcacctccgctcccacccggcgcctcggcgcgcccgccctccgatgcgctcagcggccgcagctcctcggagtcccgcggtggccaccgagtctcgccgcttcgccgcagccaggtggcccgggtggcgctcgctccagcggccggcgcggcggagcgggcggggcggcggtggcgcggccccgggaccgtatccctccgccgcccctcccccgcccggccccggcccccctccctcccggcagagctcgcctccctccgcctcagactgttttggtagcaacggcaacggcggcggcgcgtttcggcccggctcccggcggctccttggtctcggcgggcctccccgccccttcgtcgtcctccttctccccctcgccagcccgggcgcccctccggccgcgccaacccgcgcctccccgctcggcgcccgcgcgtccccgccgcgttccggcgtctccttggcgcgcccggctcccggctgtccccgcccggcgtgcgagccggtgtatgggcccctcaccatgtcgctgaagccccagcagcagcagcagcagcagcagcagcagcagcagcagcaacagcagcagcagcagcagcagcagcagccgccgcccgcggctgccaatgtccgcaagcccggcggcagcggccttctagcgtcgcccgccgccgcgccttcgccgtcctcgtcctcggtctcctcgtcctcggccacggctccctcctcggtggtcgcggcgacctccggcggcgggaggcccggcctgggcagaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgcatgtcccaaattaccatacaacaaggagacaagcccttctttctactttgccatttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactgaaaaaaaaaaa

SEQ ID NO: 2 - DNA, 151001 bp, Homo sapiens
  source 1..151001: mol_type = genomic DNA
tcccaaagtgctgggattacaggcgtgagccaccacactggccaaaacttgttcttaagattgtattctgggaccttgattccaatcagagaaaagtgattgtatttttttatttttattttttttagataaagtttcgctcttgttgcccaggctggagtgcagtggtgccctctttggtcactgtaacctccgcctcctgggttcaagcgattctcctgcctcagcatcctgcgtagctgagatcacagatgcccaccaccacgcccagctaattttttcgtatttttagtagcgatggggtttcaccatgttggccacgctggtcttgaactcctgacctcaggtgatccatccgcctcggcctcccagagtgctgggattacaggtgtgagccaccgcgccaggccaagtgtttgtatttctattaaagaaagaatataacgggacaccattgacgacctgctccattgcaggcctccttgctgttcctcagactcccccctcagagcctttgccctcgctgtgccctccacctggagcgtttctccccaggatcctcatgcccatgctcatttgggtccctgccccatgtcaccctctccaggagcttcccctcacagcagccctggcctgtaccacagccgggtacaggtatttttttgtttcaactggttttttagttccagtttcctttaggttactttatttatttatttatttatttattttttgagacggagtctcgctctgtcgcccaggctggagtgcatgatctcggctgactgcaacctccacctcccggattcaagcaattctcctgtatcagcctcccgagtagctgggattacaggcgcccaccaccacacccggctaatttttatatttttggtagagacggggtttcaccatgttggctaggctaggttaatttttaaagggttttgcaatggtcccttgatctactttttaccttagatgggaaataaaactgatttcctacattggcagaatacaatgatcatttttgcctggactatctaggaggttaatttcagttggactactgaaaactgctggttcaatcattctccacgtttatctaagtctttacctttatctggacagttctaggacattgaggggaattttggtgtttcttcccctattatttcctgaagtcatttcactttaaaaaacaatagattcactgctcaaaaaaaaaaaaaaaagttacctactttctacttgcttccagtttaactgcaacacattttaaaaagagtctactgtgctggctgggtaagttaaattaaaacttctaaagggtccaaggtctaaagttcgcacattgttttgaggtcggctctgtctctaccgagggagatcccattatccgtagttctaccagtcccaatcccatatatttcctttagaatctcatgaatgaggaaaaagaagttcaagtgagggaacataggttcaaatgaaggtcagatacctaaaagagttttctggtgactgtgcgcggctggggtggaaaaagtggggaaaaggtacccagatgtgggtggccccggagggttgctccactccagccccggcagggcaggacagcgcggcctgcctggtagatgccccgagccactggagcgcctactgtgtggcgggcgggggacggcaggaaaacggcaggatgctgtgtcccctgaatctggcagggttctaggtgctttacacgtagcaagacacattctccgccccaaggcactcgcagtcagtccattttctgggttgcatcaggtgggggcaaactaggtccccgcagaagtgaagatgctgaaggaatacagtaggagaagaaatgcttctctcctgtcctccacaccaggcaggccccagaggctgagaccgacacgccctccccgaagggcagacccgccttgaggaaggcggatccgggtagggaccgccgcctggccctcacccgacccccgagaaagcaacccagcgcgccgcccgctcctcacgtgtccctcccggccccggggccacctcacgttctgcttccgtctgacccctccgacttccggtaaagagtccctatccgcacctccgctcccacccggcgcctcggcgcgcccgccctccgatgcgctcagcggccgcagctcctcggagtcccgcggtggccaccgagtctcgccgcttcgccgcagccaggtggcccgggtggcgctcgctccagcggccggcgcggcggagcgggcggggcggcggtggcgcggccccgggaccgtatccctccgccgcccctcccccgcccggccccggcccccctccctcccggcagagctcgcctccctccgcctcagactgttttggtagcaacggcaacggcggcggcgcgtttcggcccggctcccggcggctccttggtctcggcgggcctccccgccccttcgtcgtcctccttctccccctcgccagcccgggcgcccctccggccgcgccaacccgcgcctccccgctcggcgcccgcgcgtccccgccgcgttccggcgtctccttggcgcgcccggctcccggctgtccccgcccggcgtgcgagccggtgtatgggcccctcaccatgtcgctgaagccccagcagcagcagcagcagcagcagcagcagcagcagcagcaacagcagcagcagcagcagcagcagcagccgccgcccgcggctgccaatgtccgcaagcccggcggcagcggccttctagcgtcgcccgccgccgcgccttcgccgtcctcgtcctcggtctcctcgtcctcggccacggctccctcctcggtggtcgcggcgacctccggcggcgggaggcccggcctgggcaggtgggtgtcggcaccccagccccctccgctccgggcccggcgtcccctcccccgcggcccgcgccgccgtccccgccccgtgacccgccgggctacccggggtgggctgggggccggcagcgcgggggagactcgctcgggcctgagccccgaggctcggccggtgggcgcagccggggtcctctgggattgtcaggcctgtccagcctcccgcagcatccccgccccctcccccggcggtcaagatggagggagcgggcggcctcccctccccacgcgtgttgggaggggttctcgggtagcggcgatggtcagccccggctcccccttccgcacgatcctccgcccgcagcgtggggatgctcgggcagctcctccactcccggtttaggtgtgaacgttggaggggtctggaggctgtggtggcgttttccggaacatgtccccctccatgggggacatctctggaggggagaagttagggccgcgtcccccgtgccggttaaaggggtaggcaccgggctcctccggaatcatcagggtctgtcggggctctctccccgccccctccgagtcctgggaaagatcggaggacggggtggagacaagtgggccttggcccccgcacccctctgcgttcgtgtccgaggcggcggcgggggctcccgaactcccctgaaatcgtggggctccatgtggcctccggcagcgttccaccctcccccacctggggaagggaaggggtggggagtgcccggccccgtcccggccttcctccttcccccgccagacctctccggcgcgcgggtggtggccgatccgcattgctgttcgaggccgcagtggagaaggcgcctgtggaacatcggtgggtgagggctggacccaggctggaccctggagatccggggtggcggtgctggtggcagggggcgggcaccctgcgcacttatcccaacccccgccccaatttcggaaatgctaggagagagagattgcagcaggggacgtggtcgggttcctgaaggcagaaaggcgggtgtttactagcgtctttttccctcctaagccggggttgtagtaggggctgggggctcagtgttgtcccggctaactgggtttgactcgagggtgtgtttgtgcaggagggcctgttgggggtggcgggcggttgtcagttcgtatttcacgaactaagaaaatgcttagtgttcaaagggagaaggaaacgtcaatagactccattccattgtggccggtgtccttaacttcgggagtgccgccagagcttaccaagggcacgcaagtccatttcccttgtgcctcaagtccatccgtgttgtaggcactactgtgccttctttaggcctaggccgccggcttgacggcgggtgaccggcgtcctccttaaataggcatcttgggctttggaaggtggaataagaggatttttcattcacccgagttttctttttgaaaacacattttcagcaacccatttccaaagaatttttatttacagcagaaattccccatcaagaggaatcagctggtttttaaggaattctgctgccttcaaagggggcggaaacagtcggttatttgactttacacgccccgcccccccttccccttctctgagtctgaagcatcccaaacactacttagccaaactagttcagatgaagtgatcgtttccccaagtagggtaacttcagtttccctttttcgttggcatctagcgaaaaatgaaaaaatttaaaatacaacttttatagaaaaggatgtattctgtttttactttcttaggtattaggaagagatttggcagataattcaacatgttcaaatatataaacattaaaactaaggttattaagttgcattgactactaggcttaaaaattagattataagagaatttgctcctgagtagtttgagtgatcaaagatatttggaatgttttagtaccacaaggtcttttttctgttccttgaggctttacaacaatttaaggttaatttagatttttccttgctttaagttcttttacttgagacctaaatggcagcccttattctttctgatgaataggtgaaattttgtttactgtgttggatttgtgtaatgtgaagttttattcttgaacagatcgttaatgtacttgtagaattactttgaatttgaatcactttcctgcattccttgtaaataagtttcagcttctagaatctcctcacttaggtttgtgcgtatcaacagtgaaaataagtctctgagagcaagggtgaaaaaaaatgcagcattcggtttgacaagtttcgagatagcaaaatatgcttgaaagtctggaaattcacatctgctttaagaaacatttcataatttgactttgtgtgtgtgtgtgtgaatagtttttcatgactttcagaagtgatttattttgttctttgttatatatatttttgaaggtggctgttttaggaaagataatgtaatcacaatattagaacataattttactgtaatctaattttgtgtgtgtgtgtgtgtgtgtgtgtgtgtgtgtgtgtgtgttttggatggaatctcactctgtcgcccaggctggagtgcagtggcctgatctcagcttactgcagtctctgtctcctgggttcatttaagtgattctccagcctcagcctccccagtagctgggattacaggttcgtgctaccacacctggctaatttttttgtatttttagtgaggacgggattttgccatgttggccaggctggtctcgaactcctgacctcaagtgatccgcctgccttggcctcccaaagtgctgggattacaggcgtgagctactgcccctggccaatttttgtatttttagtagagatggggtttcaccatgttggccaggctggtctcgaactcctcctgacctcaagtgattcgccagcctcggcctcccaaagtgccaggattacaggcaggaatgagccactgcccccaaccatcagtctaattcttatttttgctttttaccttttcatttttatgtagtagaggtgattgtgtatgttattttgtagttagcttttttcccctgaacgttgtattgtaaatgtaaatttttttttttttttttgagacagagtctcggtgtttgcccagtctgaagtgcagtggtacgatctcagctcactgcagcctctgactcctgggttcaagcgattctcccacctcagcctcttgagtagctggggctacaggaatgttccaccacgcttggctaatttttgtatttttggtagagacaaggtttcaccatgttggccagtttggtctcgtactaccgacctcaggtgatgcgcccgcctcggcctcccaaagtgctgggattgcaggcgtgagccactgcgcccggctgtaaggtttttacttaaccattctattgttgggaattgggtttccacttttttgttatagatagtggtgcagtgaacatttttaaatagctttttgcttcagtgtaattatttccttagagaaagttaccaagagtggttttactagttcagagggcttcaggatttttatggctcttgctagcggtgctctattattctttagaagacttgtattacttccagtgtcaagaaggttgctcttccatggaatggtttctttgtagtttgtcaaatattgtggggaatttttaaaggaaaaattgcatttttactgtcaagtgcatatattattaagtgcttttgttagttactggattattgatatttgagtttaatttggttcctctgaggatttaataaggtaatatatgtgaagatgttttgaaacctgtaaccattattattaatgagggtacttggtttatctgtcgtgctgatagtactgagtaaagtgcaggaatgaaattcctgaggaactgttctaaagctttgttgttgttgttaacctttctttttcatctgaaagtgttttttattagctgctagcctatgaccaagttatttttggtaacttttttgtaatttcatggcactattgggaattttcgctggttgactcttcttcttctacattcccttccccattaaaaataaaaatatggatttacaattgttactctattcctaaacctaaataatatgacattagaattgcttgggatacaggattcagtctgaataaaatatttttcttttagtgattttcagcttagtatttttactgcttctttctcttgaggcattgcaacttaaaaattgtgctgtttagccaggcgcctgtaatcccagctacttgggaggctgaggcaggagcatcacttgagcccaggaggcggaggttgcagtcagctgagattgtgccactgcactccagcctgggagacagagtgagactctatctcaaaaaaaaaaaaaatgtgctgtgatttaatgtagttgttcatcatgcttccatttaaatttcagtgagactgttcatcttttgcagttaaatatcttgtagaagggcctaaaatatctacgttgaatacagctttattgaagcatctatgtacatggggtttttgggatgaatcagtgaataaagcaaacatattgtccttttggagtttacattctaatgtgactaggcagacaatgagacattaaattaccagcctatgtataatagtgtataagagctatggaattagaagaaagcagattaaaggtatagggagtgtggggaggggaatgagttacaattttaaatggattgggggaacttaattgaggagctaacatttgagcaaagatttgaaggttgggtatttagccgtttgctttttatctaggttaattagtcatgtggcttcattagtaatttataaggtttaaatggcatcatcctttgttattcttttatgtgcacattgatactaaccatctctgaagttagaccaaaaaagttaattgacattgagggtcattagaggtaaattgtagatggctattactaaccaaagagacatgttttgtttttcttttgggcttacgtattttacctaattagtttagtttttgtttcaagtatgtggagaaaataaactttttaagtttgggccaaaacttgctttggttttctttttctttttctttttttttttttaagagaaaaatgtaagcctgtagttgcttaaagattccacattctgaaacagtgaaaacatgggatcagtcatggtgttcctttttttggttaaatgtaaacttgtattttcagtgttactctaattagcaatggtttatacttctacataagggatgttaactcatattgtagctatttaatagccatatattttgacttaaaggaggatctcaaggccaggcgcggtggctcatacctgtaatcccagcactttaggaggctgaggcgggtggatcacctggggtcaggagtttgagactagtctggccaacatggtgaaacccccatctctactaaaaatacaaaaattagccgggcatggtggtgggcgcctgtaatcccagcttcttgggaggctgagggaagagaattgcttgatcccggaggttgcaatgagtgcggaggttgcagtgagctgagatcatgccattacactccagcctgggcaacagagcgagactctgtctcaaaacaaacaaacaaacaaaaaaaggaggatctcatttttttgtcctaaatagctacagccgtgttagaactgtcaccttagcaaagtattgtttttttactttgaaacgaattttaaggttttagaagattgttctctagaattacaattttctgttttgactagtgatagtattttgatgttgtgtaaatagttgagcatgaacaaaaccctatttttttttttagctatttcaagtgattgtgacaacttcaacggagatgtaaacagtttattaacagtcacacctattatcttttttttttttttttttttttgagacggagtcttgctctgtcgcccaggctggagtgcagtggcacgatctctgcttactgcaacctttgcctcccgggttcaagtgattctcctgcctcagcctcctgagtagctgggtctacatgcgcacaccaccacgcctggctaatttttgtatttttagtagagacagggtttcaccatgttggctagaatggtctcaaactcctgacctcaggtgatccacctgcctcagcctcccaaagttctgggattacaggcatgagccaccgtgcttggccgctgccgtatctttttaaatgaaagtacttgtgttttttttgtttttttccaaaggatatctgggtcatctatgatgttactgttaccatctaagggtttttttgtttgtttttgagacagagtctctgtcgcccaggctggagtgcagtggcgtgatcttggctcactgcaacctccgcctcccaggttcaagcaattctcctgccttagccctcccgaatagctgggattacaggcacccgccaccatgcctggctaagttttgcatttttagtagatatggagtttcaccatgttggccaggctgctcttgaactcctgacctcaggtgattcgcttgcctcggcctcccaaagtgctgggattacaggcgtgagccacccccgcccagcctcatgagctaaggtgttttttttttttttgagacagttttgctctttcccaggctggagtgcagtggtgcaatctcagctcactgcaacctctgtttcccgggttcaagcgattctcctgcctcagccttctgagtagctgagattacaggtgcctgctaccccactcagctaatttttgtatttttagcagagacagggtttcaccatgttggttaggctcatctcgaactcctgaccttaagcgatccacctgccttggcctcccaaagtgctgggattataggcatgagccaccgtgcgcagcctaccctgtctcttaaaaaacagtaacaacaacaacaacaacaaaaaatcctaaatcttaaaaatggaaggcaaaaactctaagctttgagagattaggggacttgcccaaagcaatatttgtaggattttattacacctctccctttatttatttttttagagtcaaggtctccctctgtcacccaggctggagtgcagcctcaatctatggggccaagcatttctcctgtcttagcctcctgagtagctggaactacaggtgtacaccagctggctaacatttaaattttttgtagagacagggtcctgccatgttgcccagattggtctcaaactcctgggctcaagtgatcctcctgcctcagcttcccaaagtgctgagattacaggtgtgagccactgcaccgagccccctccctttatttttatttttaaattttaagttctggggcccctcccttgaaataaatagaaacgtaatatatacacaagatcatgctgtgtattttaaggcaatggtcctcaacctttttaacactagggaccggttttgtggaagatggtttttccataggggcaggggatgattttgagatgaaactgttccaccggccgggcacggtggctcacgcctgtaatcccagcactttgggaggccgaggcgggcagatcacgaggtcaggagatcgagaccatcctggctaacatggtgaaacccccctctactaaaaatacaaaaaaattagctgggcgcggtggagggcgcctgtagtcacagctactccggaggccgaggcaagagaatggcatgaaacccgggaggcagagcttgcagtgagctgagatagcaccactgtacttcagcctgggggacaaagtgagactccgtctaaaaaaaaaaaaattgttccacctcagatcattatgcatttgttagattctcataaagagcatacaacctacatctcttgctatatgcagttcccagtagggtttgtgcttctataagaacctaatgctgcacctgatctaacaggtggggctcaggtgctaatgctcacacagctcctgttgtgcagtctggttcctaacaggcctgtttttttttttttaattagatggagtctcgctctgtcaccaggctggagtgcagtggcacgatctcagctcactgcaacctctgcctcccgggttcaagcgattctcctgccttagcctcccatgtagttggtactacaggcgcacactgtgatgcccagctaatttttgtatttttagtagagacggggtttcaccatgttggccaggatggtgtcgatctcctgaccttgtgatccgcccaacagcctcccaaagtgctggaattacaggcgtgagctgctgcgtccggccccctaacaggcttgttttatggaatacagtcacggacagtacttgcccttcaggatatctttttgtaaccttgattttggcttgctaaaataggaggtctattttcttttctttgtttttaatgtatgtggttctgtacttacgtggtgtgaaatctacataaatgttaaatccttggttatttatttattttgagacagagtctcactctgtcacccagtctggaaagcagtggcataatctcggctcactgtaacctccacttcccaggttccagtgattctcctgcctcagcctcctgagtagctgggattacaggcatgcaccactacacctggcaaatttttgtatttttttttagtagagatggggtttcaccatgttggccaggctcgtcttgaactcctgaccttaggtgatctgcctgtcttggcctcccaaagttttgggattacagcatgagccactgcgcctcgccttatttttttgagacaggttctagctctgtcacccaggcgggagtgcagtggtgccatcatggctcattgcaacctcgagttctcaggcccaagtgatcctcctatctcagcctcctgagtagctgggaccacaggcatgcgccactatgcccagcaaaatttttgtttcactctgttgcctagggtggggtgcagtggcaggatatcggctcagtgcaacctctgcctcttgcgttcaaatgattctcatgcctcagcctcccgagtagctgggattataggcatgcgccactacacctggctaatttttgtattattggtagagatggggttttatcatgttggccaggctggtctcgaactcccgacctcaggtgatccatataccttggcctcctgaagtgctggaattacaggcataagccactgcgcctagcttttttgtttgtttttattttgtagggacagagattttacctgttgcccaggatggccttgaattcctgacctcaaacaatttgccctccttggcctcccaaggtgctgggattacaggtgtgagccactatgcctggctggttttttaaattattattattgtttgtgtgtgtgtgttgcaggatcttaccctgtcacccaggctggaatgcagtgatgtgatctcggcttactgcaacctccgcctcctaggttcaagtgattgtcctgcctcagcctcctgagtagctgggataacagctgtgtgccaccatgcctggctaatttttgtatttttagtagagatggggtttcatcatgttggccaggctggtctcgaactcctgaccttagatgatccacccgcctcgtcctcccaaagtgctgggattacaggtgtgagccaccgtgaccagtttggtttagtttttttttttttttttttttttttttttttttgagaaatctcgctctgtcgcccaggctagagtgcggtgacacaatctcagctcactgcaagctccacctcccaggttcatgccattctcctgcctcagcctcccgagtagctgggactacatgcgcccgccaccatgcccggctaattttttttatgcattttaagtagagatggggtttcactgtgttagccaggattgtctcaatctcccgacctcttgatctgcccgcctcggcttcccaaagtgctgggattataggcatgagccaccgcgtccggcctggtttggtattttttttatgagtctgggttgtttatgaaaacttgtcacagctgttaaccttaactttttttttttcttttttttccgagacggagtctcgctctgtcacctaggctggagtgcagtggtgcgatctcggctcattgcaacctctgcctcccaggttaaagcgatttttctgcctcagcctcctgagtagctgggactgcaggcacgcaccatctcgcctggctaatttttgtattttagtagagatggggtttcaccatattggccaggctggtctggaacttctggcctcaagtgatccacctgccttggcctcccatgcctggcaaccttaactttttatttgctggtaattatttgtgtttgcattcatgtgaaaatttgaaattctcattaacatttaaagattcttacatagattgcttgtaattttaaccctgaagttgtgtcaagtgactttacaatgtcaatttgttttatttatttatttatttatttatttatttatttttgtgataggatctggctctgttgctaaggctggagtgcagtgttgcaaatacggctcactgcaacctctgtctcccgggttcaagccatcctcccacctcagcctcccaagtagttggaactactggtgcgccccacagtgcctgcctagtttttttgtattttcagtagatgtggagttttgccatgttgatcttgaactcatggcctcgagtgatccaccccacttaggcctcctaacatgctggtgttacaggtgtgagccactgtgtccagcccgaaaatgtcagtttcgtgccatgattaatagctaactacattttgggaatgtaataaaatttcattctataatgaagtctttgtaaaactcattagttgtggtatgaggcttgtcggcaatataagtgaacgtggtttatttttattaactgtatcagaactttagaatgttggtctcctgaaaccattgccttgagaggctttattgaacagtgttgccaatgatcagtttttttttaaatttccttttttttgagactgagtcttaccctgttggccaggttggagtatagtggtatggtcatggctcactgcagcctcaacatcctgggctcaagcagtcctcctacctcagtctcccgagtagctggaactacaggtgtatgccaccatgcctggcttttgtatattttgtagagacagggtttagccatgttgcccaggctggtctcaaactcttaaattcaaatgatccacccacctagttttcccaaagtgctttaattacatgtgtgaggcaccgtggctggccaggtcaaatatttttcattgacgtttttcatattgctttttaaagtcatgttaaaatattcttaataatttttctaagtggaattaatcttgattataattttagttttttataaagggcgggttttgaaacaagtactgcatttttcttttcgggtttataaacatttgctgtggactttgtgcagttaactattttcattcctgaaacacatttcgaaatcaggaattgaagactaaatgtcttttcactgaagcttgagcagattttagaaaggggagttcttttttttttttttttttttggtagaaatgggggtcttgttatgttgcccaggctggcctccaacttctgggcttaaactgtcctcctgctttagcctctggtctggagagttctttatggcctctttgagaacttttactttacacatgattctatctagctttcttttctgatgtacatattggcagcaagtagaaaagcaatgttttcagaggcagatatattaacagcaatgagaaataacagtagcgtgatagaaagttgaaagacttagctgggtgcggtggctcacgcttgtaatcccagcactttgggaggccaaggagggtggatcacttgaggtcaggagttcgagaccagtctggccaacatggtgaaaccctgtctctactgaaaaacagaaaaagggccgggcgtggtggctcacccctgtaatcccagcactttgggaggttgaggagggcggattacaaggtcaagagattgagaccattctggccaacagggtgaaaccccatctctactaaaaatacaaaaaaattaaatgggcgtggtgatgtgtgcctgtagtcccagctactcgggaggctgaggcaggagaattgcttgaacccgggaggcagaggttgcagtgagccgagatcgggccactgcactgacgacagagggagactccgtctaaaaaaaaaaaaaaaaaaaaaaaaccagacttggggctgggcgggcgcctgtaatcccagctacttgggaggctgaggcaggagaatcgcttgaacccgggaggtgaaggttgcagtgagctcagattgtgccactgtgccccagcctgggccacagagcagagtgagactctgtctcaaaaaaaaaaaaaaagtttggaagactggtggctgggcatggtggctcacacctgtaatcccaacactttgggaggctgaagcaggcagattacctgagcccaggagttcaagtccagcctgggcaacacagggaaaccccatctcaacaaaaaatattaatacaaaaaatttagccagtcatggtcgtgcacttctgtagtctcagctacttgggaggctgaggcaggtggttcacttaagtctggatgtcgaggtgagccatgattgcaccactgcactccagcctgggcgttaaaatgagaccttatctcaaaaaaacaaagcaaagagcctgggaactactaaaatgggaactactaaaaaacagacacaagagctcaacaagtataccattctgggaggtttttttttttttttttttttttttttttgagatggagttttgctcttgtcacccaggctggagtgcaatggcgccatctctgctcactgtagttccgcctcccaggttcaagcagttctcctgcctgactcctgagtagctgggagtacagatattggtcacacaccgggttaatttttgtatttttagtagagacggggtttccccattttggccaggctggtctcgaactcctgacctcaggtgatccgcctgcttcagcctcccaaagtgccgggaccacaggcgtgagccaccgcacctggctttttttttttgacatagaatcttgttctgttgcccaggctggagtgcaatggtacaatcttggcccactgcaacctctgcctcccagcttctagcgattttcctgcctctgactcctgagtagctgggattacgggtgcccgccaccacacccggataatttttgtatttttagtagagatggggttttgccatattggccaggccggtcttgaactcctgacctcagatgatccacctgcctaggcctcccaaagtgccgggattacaggcgtgagccaccactcccggcctgggagttttgactgtaagtttatagctgtatatcttaggccctaagggcattactgttttatagcacagtgtagttagttaatgtgctcataatggtgactcataacaccaggttaaatgattttttatatctcccaaagaagtatttttcaatctgcagatcatgaccccttagtagattgtgaaacacattagtggattatgacaagcatttttagaaaaatgaaaaagaataagaagtgttaggatgcattgcattattgaaataattgtttttgagatggagtttcgctcttagttgccgaggctggagtgcaatggcccgatctgcctcccgggttcaagtgattctcctacctcagcctcctgagtagctgggattacagacatgctccaccatgcctggctaattttgtatttagttttagtagagatggggtttctccatgttggtcaggctggtcttgaactcctgacctcaggtgatccacttgcctcggcctcccaaagtgctggggatacaggcatgaacccctgtgcccggcctaatttttgtatttttagtagagatggggtttcaccatgttggccaggatagtcttgatctcttgacctcgtaatctgcccacctcgactcccaaagtgctgggattacaggtgtgagccactgcacccagctgccaagaattgttttaagctttggtttgagttaatgtatatataccgcattgtaattcaaaatgtaatttttggccaactctgggcacattgcctatggactagtcctgctctgccacgagcagcaacagttcaatgaattttttttttttttttttttttttttttttttttgagacagggtctctgtcaccaaggctagaatgtagtggtgcagtctcggctcactgcaacctctgtttcctgggctcaagcgatcctcccacctcagcctcctgagtagctgggagtacaggagcacgctaccatgcctggctaatttttgtattttttgaagagatgaggttttgccatgttgttcaggctagtcttgaactctggagctcagatgatccacccaccttggtgtccagaaatgctgggattacagggatgagccaccgtgcctagccaaaaatttttttttaagtaattttttattgatatagtcaaaaaagttactgctttagagccagagaaacgcagtaaaaggattgagaaagagttttgaggttatatctaagctagggttgtcagatttggcaaatagaaatacaggacactcagttaaatttgaatttttgatgaacattgaccagttttttagtataattgtgtattaaattgcatagaaaaaagttatttatctaaagttgaaatttaactgagcatcttgtattttatctggcaactccagtctaagctggaatcatggttcactgttttttttttttttttttttttttgagtcggagtcttgctgtgttgcccaggctggagtgcaatggtgcgatcttggctcactgcaacctccacctcctgtgttcaagtgattctcctgcctcagcctcctgaatagttgggattacaggcacccaccaccatgcccagctaatttttatatttttagtagagacggggttttcgccatgttgttcaggctggtcttgaactcctgacctcaggtggtccgcccacctgggcctcccaacgtgctgggattacaggcatgatctaccgtgcctggccatggttcactcttcagtaactaaaatttaagctctatgaaagcaggaactttgttttgttcactattgattgtatccctatttcttgaatggttggcacttaactgcttggtcacatgtttgaatgggcaagttactcagccactctcaggcttagtttatttacctattaaaagagaaagaatatcttccttggctgggcgcggtggctcacgcctataatcccagcactttgggaggctgaggcgggtggatcacgaggtcaggagatcgagaccaacctgggcaacattgtgaaacctcatctctactaaaatagaaaaaattagctgggcatggtggtgcgcatctgtagtcccagctactcgagaggctgaggcaggggaatcgcttgaacccaggaggtggaggttgcagtgagccaagattgtgccactgcactccagcctgggcgacagaacgagactctgtctccaaaaaaaaaaaaaaaacaaacaaaaaaaaaaactgagatactggccgggcgcggtggctcgtgcctgtaatcccagcactttggaaggccgaggcgggtggatcacgaggtcaggagatcgagaccgtcctgcctaacatggggaaaccctgtctctactaaaaatacaaaaaattagccaggcgtggtggcgggcgcctgtaatcccagctacttgggaggctgaggcaggagaatggcgtgaacccgggaggcagagcttgcagtgagcggagatggtgccactgcactccagcctgctgggcgacagagcgagactccgtctcaaacaaacaaacaaacaaacaaaaaaactgagatactaaagtcttaatattttctgtttttatgtatttattttttgagatgggatcttgctgtattgcccaggttggagtacagtattgtgatcatggcttattgcagcctttaactcctgggttcaagtgatcctcccacctcagcctcctgagtagctgggaccacaggcacatgcaacatcacaccctgcagttctttttttttttttgagacaccgtctcgctttgtcacccaggctgcagtgcgtggtgcaatttctgctcactctaacctccacctcccgagttcaagcagttctgcctcagcctcctgagtagcttgggaccacatgtgtgtgccatcatgcctggttaattttttgtatttttagtagtgacagggtcttaccatgttgcccaggttggtctcaaactcctgagctcaagtgatctgcccgccttcgcctcccaaagtgtctgcgccctacaatttaaaaaaatttttgtagagacagtctcactgttacccgggctggttttgaacttctgccctcaagtactcctcttgccttggcctcccaaagtattgaaattaaggccatgaggcagcacacccagcctaaattcttcttatgttctgttcttggcacatagtagatgttcaacaatgtagagtcaaacgcatttggagttggaatggctctggtgttttttttttttttttaaaccagaaacacgtgcagtttattgaatgccattgtagaaaagtgtgtgaggataaacggctgatagagaacttggctctgggggcagggcgaggaatggagggtggatggagtacatgggaatcagatcacgggcagagctcctggcctagataatgcctcctgatctgttgatagacttgaaagatcaacactgggatgatgctgagcagaatggtcgtaatgatgcgcacaatcagggcccagatgttcaggcacttggcggtaaaggcataggcctgggccctgatcaggtcgccaaccatcttcttgtccctagacttcacggagtaggccaatgctatgaagcccaggcagcaggagttcatgaagtgggtgttgaacagggaccagacgacatggtcgggcacggagttctcgctgtggatggggatcacggtggacattgggggagcagggttgtggggtgcccccagcacagccacctcttgctcctccttgagcatctcatagttagggggatggccgatgttggcaggagtgaagaggtttggacattgtggttcatggtgtccagggaagaccagctgtggtcgggttgctggggtggttctcagtgggcccctccctttccctggtagtttggatttctctggctctggtggttttttagtactcattctatttacgggtgaagaaattgagaccaagagggttatttaccagagtatctcatcattggctgcataactggcattagaatctgatgtacttttatttctaatacatttctttttttttttttttttttgagatggagtctcgctctgttgccgagcctagagtgcagtggggcaatcttggctccttgcaacctccacctcctgggttcaagctattcctgtctcagcctcccaagtagctgggactacaggcacctgccaccacagccggctaggttttgtattttagtagagatggggtagcaccatgttggccaggctggtctcgaactcatgacctcaggtgatccacctgcctcggcctcccagtgctgggattataggcatgagccaccatgcctggcctttctttgtcgtttcctttctttctcttcatccctcctctccttttttcccctccccgctgcctcctcctgtcttcccttctttccttcctttctctcctttttattttttcctttctttttctttctctgtctctcccaacccttcctctctccctccctccctccccttctctctccccccctccctccccttctctctccccctccccttttgttcctaagagacagggtctccttatgttgctgaggctgaccttgaactcctgagcccagatgattctgcctccttagtagctgggactacacccacctcccgttccgttgtcatcttttttttttttttcttttttggagacagaatcttcctctgttgctcagggtggagtgtagtggcacgatcatagcttactgtaactgtgtaacctcgaattcttgggctcaagcaatcatcccatcatcccacctcagcttgctgagtacctggggctacaggtgtgtaccaccatgtccggctaattacttttcttatttttaatttttcggagataggatcttgctctgttgcccaggctggtgtcaaactcctgggctcaagtgaaactcttgccttggcctcccaaagtgttgggagggattacaggcatgagccactgcacccagcctcctctttcttcccatttaactcctaaccacaccgaactttctgtctgcagagaggagcattggtcagcagttcacaaaatggctaggtgtgatggcgtgcacccatagtcccagctacttggggagctgaggtgggaggatcgctggagcccaggagttcaaggccctgggcaacacagcaagaccttatctctggctgggcccagtggctcacgcctgtaatcccagcactttgggaggctgaggtgggtggatcacctgaggtcaggagttcgagaccagcctggccaacatggtgagaccctgtgtctactaaaagtacaaaaattagccaggcacggtggcgcgctcctgtaatcccagctactcgggggggctgagacaggagaatcacttgaacccaggaggaggaggttgcagtgaaccaagaacacgccactgcactccagcctgggtgacatagtgagactcttatctcaaaaaaaaaaaaaaaaggtcgtctgtactattgcatgttagtagtttctttctgcttattgttgagtagtagtctattgtatgcatgtaccagtttgttcatctagtggtggacattgagttagcaggttttggctattaaaaataaagctggaggccgggtgcgatgtctcacgcctgtaatcccagcattttggaaggccgaggcaggcggatcacctaaggttgggagtttgagaccagcctgaccaacatggagaaaccccatctctactaaaaatacaaaattagccaggcgtggtggcgcatgcctgtaatcctagctactcaggaggctgaggcaggagaatcgcttgaacccgggaggcagaggttgtggtgagccaagattgcaccattgcactccagcctgggcatcaagagtgaaactccgtctcaaaaaaataaataaataaagctggtatgaatatttatgtacaggttttgtgtgaacatatgattttatttctcttggttggaatgcatagaaatgagattgctgggttttgtggcaagtgtttatttttccagggtacatataatcctgtgagtgtttatttaattttaaaagtaattgctaaactgtttgctaaagtgactgctatattttctttccctagcagtgtatgaatttttttttgaggcagggtcttgctctgtcacccagggtggagtgcagtggtgcgatattgtctgactgcaacattgacctcctgggctcaagtgatcctcctgcctcagcctcctggctgggaccacaggcatgtaccaccacacctggtagtttgctttgatttttagtagagaagaggtctcactatgttgccctgggtggtgttgaactcctgggctcaagtgattcatctacctcagcctcccaaagtgctgggattatagatatgagcccctgtgcctggcctcattgtggttttaatttgcatttccctaatgcccagtgatattgagcattttttcatgtgtttatttgacattcataccatctttggtgatgagaaactatgtttatgcattgcttaatgatggggatgtgttttgagaaattttttcggtgatcttatcattgtacaaatatagagtttacttacacaagcctagatggtatacctactagacacataggctgtcgtacagagtattactcttaggctacaaatctgtatagcatgttgcggtactgaacactgttggcagatgtaacataatgttaagtatttgtgaatctaaacatatctaaacatagaaaaggtgagtaaaaatacagcgtaaaagataaaagtggtatatctgaataggtcacttaccatgaatggagcttgcaggacaggaagttgcttgggatgagtcatttatcagtggtgtgtgaatgtgcaggcctaggacattactgtatgctactgtagacaaacactgaacagttaggatacactaaattgataaatatctttcttattttgttttttgagatggagtctcgctctatcgcccaggctggagtgtagtggcgtgatgttggctcactgcagtctctgccttctgggttcaagcgattctcctgcctcaacctcctgaatagctgggattacaggtgcgtgccaccacacctggctaatttttgtatttttagtagagacgggggtttcaccatgttggccaggctggtctcgaactcctgacctcaggtgatccacccgccgtggcctcccaaagtgctgggattacagatgtgagccaccgcacctggccagagatgaggtcttgctgtattgcccagggcgttgaactcctgggctccagcaatcctcccacctcagcttcccacgtagctgggactgtgggtgcacgccatcatgcctagccgttttgtgaactgttgaccaatgctcttttctgcagacagaaagttcactgtggttaggagttaagacttttaacctctgacctcaagtgatctgcccaccttgacctcccaaagtgctgggattacaggtgtgagccatcacgcctggtcaaaaatatctttctttaagagtaaatttaccttaacttactggttgatcattgtatataggtctgttgttaattgaaacatgcgggccgggcccggtggctcatgcctgtaatcccagcactttgggaggccgaggcgggtggatcacaaggtcaggagatcgagaccatcctggctaacacggtgaaaccccgtctctactaaaaatactaaaaattaaccgggtgtggtggcgggcgcctgtaatcccagctactcgggaggctgaggcaggagaatggcgtgaacccgggaggcggagcttgcagtgagccgagatcgtgccactgcactccagcctgggcaacagagcgagactctgtctcaaaataaataaataaataaataaataattgaaacatgcggtgcatgtgtttatttgcgatctgacttgtttggaaatatttgcattatcttccttctagatttagagcatcttgacagtaggaacaagtgttttgtacaactttgtatgcttagtaagttatcaattaacttgtcgtggccaggcgcagtggctcacgactgtaatcccagcactttgggaggccgaggcgggcagatcacctgaggtcaggagttcgagaccagcgtggccaacgtggtgaaaccctgggtttgtttgtttgtttatttatttatttattttttggagacggagtctcgctctgtcgcccaggctggagtgcagtggcgtgatctcggctcactgcaacctccgactcccaggttcatgccattctcctgcctcagcctcccaagtagctgggactacaggagcccgccaccatgcctggctaatttttttatttttagtagagatggggtttcgccgtgttatctgggatggtctcgaactcctgactttgtgatccgcccgcctcggcctcccaaagttctgggattacaggcgtgagccaccacacctggcctaccctgtgtttattacaaatacacaaattggccatttgtgcgtggctcatctacagtctcagtgactcagaaggctgaggcaggagaatctcttgaacccgggaggcagaggttgcagtgagcagagatcgtgccactgtacttcagcctgggtgacagagtgagactgtgtctcaaaataataataataatttgttgaatatgtgactgttggtttaatttttatttttatgagatggagtctcactctgttgcccaggttggagtacagtggcgtgcagtggcgcaatcttagctcactgcaacctccgcctcctgtgttcaggtgattcagcctcccaagtacctgagactacagacgtgcactaccgtgcctgactaatttttgtatttttagtagaaatggggtttcaccatgttggtcagcctggtctcaaactcctattctcaagtgatccgcctacctcgaccttccaaagtggcggaattataggtgtgagccgtggtgcccggccagactattggtttggtttggtgtgatgttatgttatgttatgttatgttatgttatgttatgttatgttatgttattttaagacagagtttgtctcttgtcgcccaggctggagtgcagcggcatgatctcggcttactgcaacctccgcctcccaggttcaagtgattctcctgtcttagcctcccaagtagctgggattacaggcgcccaccaccgtgcctggctaatttttgtatttttagtagagacagggtttcaccatcttggccaggctgttctggaactcctgacctcatgatccacccgccttggcctcccaaagtgctgggattacaggcgtgagccactgcgcctggctgactattggttttattattaagcagtagtagttgaccctgtcatgtagaaagcatggcatttataggcataccacgtttaatttcctcccctttttttatttttggagtacctcctgcttgtgaggcttgggaatacagtagtgaataagccagatgaggtctctctctttttggagcttatgtggtagtatagactaggcagaaagttctcattgcccctgccaccttatggcattgaggtgtttgagatgctgatgtttacttctgtctcataaaatcttgaaaggagttcttttagatgaagaggaaaacaaaatcagaagaatgggcctgggtcatgtctgtaaacctccccacgtcatggggaggctgaaatgggaagggccaggagttcaagaccaggctgagaaacataacaagaccccatctctacaaaaaatattttttaattaatgggggatggcagcacacacctgtagtcgcagctactacgaggctgaagcgagaggattgcttgagctcaggagttaaagattgcaggagctatgatcacagcactgcgctccagcccctcttatcagcagtctggtatgttgctaagggtcttgttctttttagtgcttcagggacagccactggctatgcccagaaataagtatgtttgagaagctttctgacctcagcttgaaaaattgattagggtcataattaaaaagggagggaaacaggattgagtgaaccggacgctaccgtgagtttattctcccagggcatacataatctcatgtgattaccacatagccctgttagataatctgttatcctgtcctcattttacccatgaggaaatgaaggcccagagaggttaaatgacctattcaaattcactcagaaggtggcagagatgagttactatcattgtattttggatctctggaaagaaagaaaactagtgatggtattaaaaaatgttattaatagtttcttttaatcaaccaggaacttgagtcactagcttctctgggtgaaggactatacttcaacagtatgaaaaacggaaaagaaaatgaggaattttggctgggcacagtggctcacacctgtaattctagcactttgggaagccaagggaggagggtcgcttgagctcaggaattcaagatcagcctaggcaacatagtgaggccccatctctacaaaaataaattagctgggcatggtggtgcatgcgtatagtctcagctacttgggaggctgactcaggagggtcacttaaacccaggaattggaggttgcagtgagctatgattgcgccactgtataccatcccaggcgacagagtgagaccctatccccccaccgccaaaaaaaagaaaagaaaatgaggaatttacatttgtgacagatacggaattcagggaatttagttgttcatagtctataaatgctataagaagtctccatacctttttttttttttttttttttttttggagacagagtcttgctctgtcgcccaggctggagtgcagtggtgcgatcttggctcactacaagctctgcctctcgggttcacgccattctcctgcctccacctcccgagtagctgggactacaggtgcccgccaccacgcccggctaatttttttgtatttttggtagagatgaggtttcactgtgttagccacagatcccgacctcatgatctgtctgcctcagcctcccaaagtgctgggattgcaggcttgaatcaccgcacccggccggaagtctccatactttttaacccaatctaaaatggtaaggaaatatataagaatgtctatttattattaaattttttctatataaaacatttcagaaaataaagactagcatttctgagccaagtggtagtagtggccattttttctggaaaaaaaaaaaaaaagaaagaaaaaacacatttagctatctatgatgtgaaaagatgaacattttatttaggtaataaatgttatgtcataaaataccatttattgtgtgcctattaggtttcaggagagctgtgccaagagcattacttgtatatcttttaagccttacaacagcccagcctgtcaggctggtagtgccatatctgttttacagatgaggaagtgatggattggagaaattaaggaaattgcctttaggtcaaagagataggaagtgacaaagctgagatttttaaccttgtgagatttcaaagtctttgctttttaataactgttccattgcttctaatatagagatatgacaaaaacaagtaaaaatcagtgaagaaggctgggagcagtcgcttatgcctgtaatcccagcagtttgggaggccgaggcgtgtggatcgcctgaggtcaggaatttgagaccagcctggccaacatgacaaaactccgtctctactaaaaatacaaaaaagttagccaggcgtggtgacaagcacctgtaatcccagctactcagtaggctgaggcaaggagaatcgcttgaacctgggaggtggaggttgcagtgagctgagatcgctccattgcactccagcgtaggcaacaaagcaagactccgtctcaaaaaataaataaataaataaataaaaataataacaataatgaagaaaacaatccggtgattattgtcagcaataaaatttcttcaatcaaccatgctttagtcctggcagttctctatcagtgagtttcaatcaaaaagtttgtttataatttttttttttttaaaattttgaaatttggaaacaacatcataaatgatggttagttttctgcagctccctattttggcagatagtctgttgttactcataattaatttgaactaaaaagtagtgttgtacgatatcatgggctgtgaatgtgtttgtgacttgatctgagaacccacacaccacttaggatgcttctgtaggaaaattagagtatggaactcacttgcccacgctttccctgtctcagtccatgttggtaggctgcaaagtctggggctagaaggacactgaacaagacttcagcagtacatgttagtcttccagagggaaggaatataatagttgagagaataattcctttcctctgtgactttaggcaaattcttggctatgctgttatttatttgggccaaacaatatcaggaggttgtacattttattcttaattactgcgatacattaattttatccatgggtttaacctagcctaccttttgctgttagacttcaactctacttgtgttgggttacccctctgcttaaaaatcaccctattcccaagcctgagggagtctaccttcaaagctttctatgacctaatccaaggcctgtcaaacttcgtaaagggccagatagtaaatttgttttttttttttgagatggagttttgctcttgtcacccaggctggagtgcaatggtgccatcttggctcactgcaacctctgcctcccaggttcaagtgattctcctgcctcagcctctcaagtagctggggttataggcatgtgccaccacgctcggctaatttctttgtatttagtagagatgggggtttcaccattttggtcaggctggtctcgaactcctgacctcaggtgatccacctgctgcggcctcccaaagtgctgggattaccagtgtgaaccaccgtgcccagcccgatagtaaatattttaggctttgcagtccatatacagtcccatttttttgtgtatgtttgcacgttttctttacatattttaaaagcccctttttttttttttttgagacagagtcttgctgtgttgctcaggctggagtgcagtggtgcaatcttggctcactgcaacctctgcctcctgggttcaggcgattctcctgtctcagcttcccgagtaactgggattacaggcacatgctgccacgcccagctaatttttgtatttttagtaaagatggggtttcgccacattggccaggctggtctcctgatctcaggtgatctgcccacctctgcctcccaaagtgctgggattacaggtgtgagccaccgtgcctgacctaaaagctctttacagtgtaaaaaatattctgagctttaagccatgtgaaaataggccatgggcatttgctgacccctaatagaactccattttacctttctgatcatgtttcccattaactcttcaaaaatatgacctccatttaaatcaagatggtctccttcctcactgcttgtggaggtccagtgcccagtgtctgcctcttgcttgctcctccatcattgttctgccattcgagatcctcatacttaccctttaagatctagcccaaattttccatgaaactaattctaataattaaaaacttcctgtagaacttaactttgtctagtacaagttagctttcttattcagtagtagcttactataaattacaagaataaaaagattaccattttccctcacactgttttgtggagaatgcctaaagttactttttctttttacaggtcagtattcctatttggcatcctaatcccctttcccaaatctgaattttgggatttgaagcttgcatttgagattatgatttgtcttccttgttgtacacaggagcagggactttacaattagtattcgcatccctgctccttcatacttcgtgatgtaaggcaagttattttcacttatgcttaagtttcttcccctgtaaaaaggggatggaagaggattaaatgaattaaacatgtaacacgcttaaagcaatgcctggcaagtaataagtgctcagtaacttttagctgttcttattagcatgtttggaaaccagtagaaactacaccagcaagttaaggttgaaaagtggtattgatgggcttggggtagtacagtatgaatggctacagtttagcgtttcattaagtttgtatattcattaattcattacacatttgatgctgtcagactaggacagagacaaagatgaatgaaacattatctctgcttccaggttacccagtgtagtagagaaggcaggcatgcagatagtttaaattggtagcactgggaggggactgccatgggtgggcagtgaagaaaaagggcttcaaaataatgagagttgagatggatcttcaaggaagataagcagttttcagtaaggccatgaagagaggaggaagttccaggcgggaagagtttgtgctaaagtacagggattgctatacacatggtgtatgtagaaaaaatttggttcacagtgtgatagaagaattggagggggtcctcactgaaagtaaggaaacacatttggaagaatatgtttcagttagaaaatgaaatgagcttaaagtaaacgctaataaggtttttaaaatgtaaaatttcaacgtatttagaaagagaacagctggatgaatcttatgtacctgtcactcagctttagcagttatcagtaaatggccaacgttgtttcagctatactcccctctcctccactgatagtcttttgaaggggaatacaattgttttgtggcctccagaaagggataagtttatgagcaacgggtagatcgttgggagagacttgagtttcctgtcaggaagcattcttggtgcataagtcagaggtgatatgaatgccgtggaagggggtggcttactgtctggagaactcgagaagatgggaatgggcactgtccagtattgtggctacttccacacatggttctttaaatttaaaattatgttgattaaaatttaaatatttcagttcctcagccatactaatcgtatttcaagtgcttagctgccacatgtgcctaatggctgcaatattggacagcatgacataggacatcttcatcattgtacaaagttctcttggacagcatgggactagagccctaagatccttttctacctgagttgtttggattttttggtgtgtctaggttggatctagttgttcatggcttcatgaccaagccttttatccctttctctagagggactcaaggggtaaaggcactgaaggggtaaaacttcatatgaagagtgtggtggtggtggtggtgttttaagacagggtctcgctctgtcactcaggctggagtccagtggcatgatcctggctcactgcagcctcgacttcctgggcttaagtgatcctcccacctcagctcccaagtaactggaactgtaggcatgagccaccacacctgcctaatttttaaaactttctgtagagacgagatttcgccatgttgtccaggctggtctcaaattcctggactcaccttggcctcccagagtgctgggattgcaggtgtgggccactgcgactggccttttttttctcctttactactctagtgtatgctggaatatgaggaaataattatattagctagcagttattaaacacttaataacataccaggcactgttttaagctatgcgatctgtatggaatattacttaatttccacaaccttatgaaaagatactattttttttcttttgagaaggtactattttcatcttcatttcatagatgttgaaattgaaacacagagagctgaagtcacaggattaaggccacagagctgagaagtgatggagccggaatttgaacccaagcaattaatgctgatattagttcttgtgtgaatggtaattgttttgaaacaatgatcctagatgattatatgaccggattaatctggcagttgttctgtgtgaatttagagttgccttcccacctcagtttcctaaaaacaaaacaaaacaaaacaaaacaaaaaaaactctagcttcactgtgtttgggttgtcatggcctaccccctcttgccacctcatttgactcaactttttagggagaaaatattcaatacgtggtataggatttccctttctaataataatgtaaacaacaacaagaagtctgaaattggaagaacaaaatgactcacctaagtgagttaaccttaagaggtggaacttgatttctagattttagttaattgtctaactgatgtactaaatattagttacttaagtattaaaacgggtagacataatagttggggagctgctgtagagggggtagtttgagaaggcttctttcaggaggtgacatttaagttggtaactaacaagaaaggggcagccatgtgaatagctggagggaagagcattcttacagttttactggaagggggttagaggtatgtggtacccttatgccaaagaaaattagttacttctatacaaccagtctgattctagaaacctggatcaatgaaatattttgattatataaaaaaatctgttacccaggtcttgttgaaatagcattagaaactactgaaggacatatagaggaggagtgttgaaaaatggtgatggatgagcagaatggtgaaaaataaaaagacatgaagctctataattatattgtatggtgacagtaccaatagagattgcatgttttttctccccagtttttttttttgttttgttttttgtttttgagacagagtctcactgtgtcactcaggctggagtgcattgtcgtgatattggcttactgcaacctctgcttcctgggttcaagcgattctcctgcctcagcctcctgagtagctgggattacaggcatgtgccaccacgcccggctaatttttgtatttttatttgagaggggatttcaccatgttggcaaggctggtcttgagttcctgacctcagataatccacctgcctcagcctcccaaagtgccgggattacaggtgtgagccactgcgcccggcctcccccagttgttgaaacaataatggaaggtaattttattcttagattatttaatgtttttcagttatcaggatgtgttagattgtttgtgtatattgttttgcttgttaattaagtaacacagtgaataagacagacaaacatacgaaaatgtacatttattttatttttttgagacagtctgttgcccaggctggagtgcagtggcccaatctcggcccactgcaacctctgcctcctgagttgaagcgattctcttgtgtcagcctcatgagtagctggggccatgggtgcacgccaccatacccggctaatttttatatttttagtagagatggggtttcaccatattggccaggctggtctcgaattcctgacctcaggtgatctgcccgccttggtctcccaaagtgctgggactacaggcatgagccactgtgccaggccatttcatttttggaacgttcttttttttttttgaaatggggtctcgctctgtctcccaggctggagtgcagtggctcaatctcagcttactgcaacctctgccttccgggttcaagtgattctcctgcctcagcctcctgagtatctgggactacaggtgcatgccaccacgccaggctaatttttgtatttttagtagagacggggtttcaccatattggtgaggctggtcttgaactcctggcttcgtgatctgcccgcctcaacttcgcaaagtgctgggattacaagtgtgagccaccacgcccggcctgtttctggaatattcataatcttttgttgtcatttcaacagtgctcacagcagcttcaccaggtgtagattccatcttaagaaaccactttctttgcttatccatgagaagcaacacctcatctattcaagttttatcatgagattgcagcaattcagttacatcttctgaccccacttctaattttagttctcttgcttttttaccacatctgcagttacttgctctactgaagtcctgaacccctcaaaatcattcatgagtattagaagcaatttcctggttgggcacggtggctcatgcctgtaatcccagtactttgggaggccaaggagggcggatcacctgaagtcaggagttcaagaccagtctggcaaacgtggtgaaaccccgtttctactaaaaatacaaaaattagcggggatgtggtggcgggcgcctataatcccagctacttgggagactgaggcaggagaatcgcttgaacctgggaggtggaggttgcagtgagttgagattgtgcccttgcactccagcctgggcaacaggagcgaaactctatcttaaaaaaaaaaaaaaagaaaagcaatttcctctaaaactcctgttaatgttgatgttttaacctcctcccatgctcatggatggcattctcagtggcatctagaatggtgaatactttttagaaagttttcaatttattttgccatcagagaatggctatgaatggcagtagtagccttacagaatgtatttctttttttttttttctttttttttgagatggagttttttttgctcttgtcacccaggctggagtgcagtggcatgctatctcggctcaccgcaacctccgcctcccgggttcaagcaattctcctgcctcagcctcctgagtagctgggattacaggcatgcaccaccatgcccacctaattttgtatttttagtagaggcggggtttctccatgttggtcaggctggtcttgaactcccgatctcaggtgatctgcctgcctcggccttccgaagtgttgagattacaggcgtgagccaccgcgcccggccgtatttcttaaataaaatggcttaaacgtcaaaattatcccttgatccctgggctatggactgattcttgtgttagcagttatgaaaacatttatgtccttgtacattcccatcatagctttttgtcaatgagaagtaattttttttttttttttgagacagaatctcactctgtttcccagcgtggagtgcagtggcatgatctcagctcagtgcatcctacaactctgaggttcaagcaattctcgtgcctcagcttactgagtagctgggattacaggcgcccaccaccacgtctggctaatttttgtatttttagtagagatggggtttcacgatgttggccaggctgggctcgaactcctggcttcaagtgatccacctgccttggcctcccaaagtgctgggattgtaggtgtgagccactatgcctggcctaattggcctaatttcaatattgttatatctcagggaatagagaggcacgaggagaaagagagacaagctgactgctggttcgtggagtagtcataacacacaacatttattaagattgctgtcttatatggaccgtttgtggtgccttaaaagaaatcagggtaacatcaacgattactgattacagattactataacagatacaataataattgtaaattattatttacaattgtaaaatacaatcttttctttattatttacaattattgtaaaatacaatctgattacagattactataacgtatacaataatagtggaaaagtttgaaaatattgtgagatttatgagaatgtgacacaggcgcaaagagagcacatgttactggaaatacggcactaatggacttgcccgactcggggtttccacagacggtcagcttgtcaaaaatgcagcatctgtgaatttcaataaagcaaagcagaataaaatgaggtatgcatgtattgccatcacatgtacactagtaaaatacgttttttttttcagtaggtggatcaacctcaaattttaatataaagcattacttaaaggagaatatggggacattcatgacatttcttatatgtacataaaacttcatgaaaataatttaatgctatccagcagtttattttagaagtactggaggctaggcatggtgtcttatgcctgtaatcccagcactttgggaggctgaggtaggaggatcacttgagttcaggagctggagaccagcttgggcaatatagtgcgaccccatctctacaaaagagaaaagaagtactggagtgttgcagctcttacagaatttgtctagcaggttttccagtctttaccagaaatgcccccatgcagaagtagtaaatactgattcatgtaaaataataaacaactttatctttcagtttttaaaagacagggtcttgtaacgttgcccagactggcctttaattcctgggctcaagcgatcctctcacctgagccttttgagtagctgagactacaggctgcacctctgcacctggctctgcttgatttttaattgttgtattgctgttgcagctatgtttttttttttcttcagtgtgaggatgggcaaactttttatgtaaagtctcaggtaataagtattttaggctctagggccatatagcttctctgttgcatatcctttttttttttttccatttcccctcaaattccttttaccataagcaactcttgaggaacataaaaatcattcttagcccagaagccagaccaaaacaggttgtgggctgtagtgtcctgacccctgatttaaagattgatagctttgaaatggaaagttttaactttctttttttttctttcccttgttctgattgggctgttaattcattaggtatttactcagtgtgtatcatatgaggcatgattcctctgctaattttggtagtggtagaaagatacttttgccaagcttggttgttaggttttcatttgtccaagagttcctgaccaagtgtgaatggatgttgaaatcaaggtgtttctttggccacacaatgtgcctttgggggctatatctatgtgcttctggtaccttcttttaattttcacaaagacactgcttgccgaccacactgttttgtctaatgtggggctatgaccccctggaagaggcatcattttctgattttcacagaagcataatatggtcaggtgatggtcctgagtagtgggtatatgacagatacactagtaattataatacagatctaaactggagagttgaaaacagcatcgtatatttgattgagataatcgaaggaagacttcctgaaaagatggcatttgagtttcaaggctgagtaggattaagtattattatttaaaaaatgccttggacaatgcattaaatagagttaacaaatcacatcacttatagtctccaattaaaaacattttacttaaacataattttagacttttagaaaaattgcaaagatattataaagaattctcctatatatctcacctgtattcttcaagtaacattttaccatattcaccttaacattttctctgtattggtaattgtatatgtaagatttaatataaaataaaaattcttattaaacatatgagagacatgatgcctctttagccctaaatacttcaacttgtatgtactaataacaagggcattctatttcaaaaccacagtacagttgtcaaaataaggaaattaataattgtgtcaaactgttattctgtttatagaccttctaatgtcctttaaaacaatcaacaaatcaacatttttctggtcaagaaccagtaaatatgtatattctacatatatatatacacatatatatacacacatatattctacatatatatgtggaatatacgtatttactccctctgtccaagaaccaatccaggattgttaccttcggttatcatgtatctttggtctcctttaatccaaagcagtttctttgtcttttatgacttgacacttttgaagattacaggttattttgtagactgtccctcaactagggtttatctgaggtttccttatgattagattcagatatttatttttggcaggaatacaacagaaatgatttgtgtgtttttctcattgcatgatatcagaaagtgcattgtatatatttatcccattactggggttgttaactttgatcacttggttagagttgtgtctactaagtttcttcactataaagttatttttcacttggtcatttcatcagtatcttgtggggagttactttgtggttatataaatactctgtttctactttcccttactatatttagcttctgtggacacttttgcctgaaacagttatttactatggtgttaccaagtagtgatgcccttttcttccatcattctgtctacatttttttttttttttttttttttttgagatggagtttcgctcttattgcccaggctggagtgcggtggcctgatcttggctcactgcaacctctgcctcccgggttcaagcagttctcctgcgtcagcctcccgagtagctgggattacagacatgcgccaccactcctggctaattttgtatgttcagtagagacaggatttttccatgttggtcaggctggtctccaactcccgacctcaggtgatccacccacctcagcctcccagagtgctaggattacaggcgtgagctgccacaccaggccttctttttctcttttaagagatagagtcctgctttgtcaccaaggctggagtgcagtggcatgatgatagttcactgcagcctcaaactcctgggctcaagtgaacctcccatctgtagctgggactacaggcacctgcataacacctgactgttttttaaaactattttagagatggggtcttgcgaagttgctcaggatggtcttgaactccgggtcttaagtggtccttctgcctcagcctctggattagttggcattacaggcatgagccattgtacctggcaagtgcatattttctttttttttttttaaggtggagtctcgaggccgggcgcagtggctcacacctgtaatcccagcactttggaaggccgaggtgggtggatcaagaggtcaggagatcgagaccatcctggctaacatggtgaaaccctgtctctactaaaaatacaaaaaattaactgggcatggtggcacacgcctgtagtcccagctactcgggaggctgaggcaggagaattgcttgaacccaggaggtggaggttgcagtgagtcaagatcatgccactgcactccagcctgagcgacagaggtagactctgtctcaaaaaaaacagaaagacggagtcttgctctgtcacccaggctgcattgcagtggcatgaactccgcctcctgagttcaagcaattcttgtgcctcagcctcccaagtagctgggattacagacatgtgccaccacacgtggctaatttttatagttttagtagaggtggagtttcaccatgttggctaggctggtcttgaactcctgacttcaggtgatccacccgccttggcctcttgaagtggtgggattatgagtgtgagccactgtgcccagccaagtgagtatttgcttatgtagtattttaattttatgatttttttttctttgagacggaggtttgctcttgttgcccaagctggagtacagtggtgccatctcggctcactgcagcctccacctcctgggttcaagccgttctcctccctcagccacctcctcctgaatagttgggattataggcgcctgccaccatgcctggctaattttttgtatatctagtagtgatggagtttgagcatgttgccaggctggtcttgaacctctgacctcaggtgatccacctgccttggcctcccaaagtgctgggattaaggcatgagccaccatgcccggccagagactgttcatttattttttttttttgaggcggagtctcgctgtattgcccaggctggagtgcagtggcacaatctcggctcactgcaagctccgcctcccaagttcacaccattgtcctgccttagcctcctgagtagctgggactacaggtgcctgccaccacgcctggctaattttgtttttgtatttttagtagagatggggtttcagcccgccttggcctcctggagtgctgggattacaggcgtgagtcagggcgcctggccaatcataccttcttttactgcattaattatggttttctttcgttcttaaaacatgtttatagtgaccacttttgaaattcttattaagtcagacatctggttatacaagcaatttctattgcctacttctttttccagtgggtggggttatactttcctgtgtcttagcttgtcgtttttttttttgttgttgaaaactggacattttaagtaatgtagtaactctggatacctcattagcctatggttgggggtggtggttgttactgttatttgcttatttgtctaatgactggctgaatgattttagtgttctatccttcttccctccctgtacagtgtgacacgtctgatgctagttttcttgggatgcagccttgggtatgcctaccatcactctagaatcacagtgattttggcatggctttgtctcttttcctgactgtacccagctgttaagctacactaattactaggtgatgctgtgtagtcatttcttggtgtccttgggggattggtcccaggacccccccgttggatataaaaatttatggatgctctagtccctcataaaatggcacagtatttgcatataccggtgcacatcctcctgtatgctttgtcatttctagattacttataatacctaatatggtgtaaacactaggtaaatagttgttatatattttttatttgtcttatttttattgtatttatttttaagtgtttttaatctcgagtgattgaatctgaggatgtgaaatctgcagatatggagggcctgcattgttttccgtggagctttgggcctaaactgctccacagactgatctgatcaaatttgcgcttctttgaagggatagtttctgagatcagtgtttgaaatttgttccaatccacagaggagtcctcccagctctctttccctagttctggccaccaaactagacaactacaatttagcacttatctccaatgattctcctcctaccaagtgcctttgaaagcatcattaactctttcataccttgttgcaaatgaaatttctttgggaagagattgtgagttttttttctcctaaattatggtgcaatataagtaatataccattttaacaattttaagtgtattaagtgttttttttttttgtagtttttttttttttgttttttgagatagtcttgctctgtcgcccaggctggagtgcagtggcacgatctcggctcactggaacctccacttcccgggttcaagtgattctctggtctcagcctccccaaatatctgggattacaggtgtgcaccaccacgcctggctaatttttctatttttagtagaaacggggtttcaccatattggtcaggctggtcttgaacttctgagctcgtgatccacccacctcggcctcccaaagtgctgggattacaggccttagccaccacacctggcctatgcattgcttttatatgtattttaaaattcataagttctcctcctatgatgtttttgtcccatgtgatttatttgttaaaccgtcatctttggccgggcgtggtagctcacgcctgtaatcccagcactttgggaggctgaggtgggtggatcacaaggttaagagatcaagaccatcctggccaacatggtgaaaccccgtctctactaagaatacaaaaattatctgggcatggtgacgcgtacctgtagtcctagctacctgggaggcggaggttgcagtgagccaagatcgtgccactgcactccagcctggcgacagagtgagactctgtctcagaaaaaaaaaaaaacaaaaaactgtcattttttatgttgcatttactgcattctggatttaaactgtgaggaacctcatggtatcagttaatatattcttccatcttaatgtttctcgtaaactggtagatctgtaaacttgattaggtctatcctattgtatcacatcagaagcagaaggtgcttttttttttttttaagggaaattgtgtgaaagtagacagaatggtaaagtgaacccctgcacacctatcacccagctttaatagttatcagctcataccattcttgtttgatttacaaccccattcatttctcccttctgtattattattatttagttaattattttttgagacagggttttgctctgtcaccaatgctggagtgcagtggcataatcacagctcactgctgtcttgacctcctgggctcaagtgatcctcccacctcaccctaccaagtagcggggaccacaggcgtgtgccaccatgcctggctagttttttattttttgtagaaacagggttttgctttgttgcccagactgatctcaaactccggcactcaagtgatcctcctgcctcagcctcctaaagtgctgggattacaagcatgagctaccacattcagcatgtaaatttctttatattaatttgactggcattttaagtcacacttgaatttcatatttggcaactattaaaagcatagagtcctggatattagtgttttgttaaacctgatctatctaatcataaatatacttaggtctaaaatatgctcttggcctttgtttattgcggttcagtatttgttactatattaaatagtaaaatatttggtttgagatactaatgaaaagattaaaagtaaagcataacttgaatggatacaaaaagaaacaagaatttagacttcagtggatttcagagaatactgcttcgatatgctaacattcctgttgggtgtccaaccgtgtcatagatcagtggaaattagtggtttctgcactttactgtactgtttttttatatgataatattttcctggttgaatgattcgttcttttgagtaaactccatggtcaaacaattactttttattagtcaaagatgtaaccacataatcactaaaaagaacagtgtgacttatttaaaggggattatgtttttaagtcttttatatagctttgtagggaggccatatgagtttaaggacagttcgtggcatttgttcaaggttttgtaacttggcatctcagcagccaccaggataccagatcatcgttctaagtaagatttaggcattttagccttcatgtacagactataagtacacccccccacacccctaccaaaactgtaaattcaaatgatgtttgaaaaagcatagaatttttgttaggcgaggtagtttattccttgtgatacagttccagagaggcagcataacctaggaatgaaaaacttagacgtggaatcagatacacctggtttaaataccagctctactgctcatgaactggatgattttggtcaagatacttgactgctgaggttcagtttcctcacctgtaaagtagaggtgatagattagacatgttgcatgtgaagtacttagtatggtgtctggttttgtagtaagatctataaaagataaattattagtcatattccttagacttcaggaatttatctctgtgccatgtttgaggcaaacagttacagaattagaatgttagaaatgaaaggaatcctagatgtcatttaattcaagtccattgttttctggatgagagaagaaagtgaggaaaagtgacagagttggagaccaagctaggactggcctcagaatgttaagagtactcttctagggatcgaccagtcgtgttactagactttttggatctgaattgtgcttttccttgaatgttttgaattttggcttgagtgttgtgattattttattaaaatgagattccagtcctattgtcatgactaatgtttatgagaaatataacatttcactttaatgatgttttttaattattctaaggggcctaatctttttcagtggaataagctttaggttgtattatattctataattcacttgaaaatagaattcatctttacttgacagccaaattttgtgtactgcatcttttctgagggagagagttggcaaggaaaggcacttgttacaacgatccacacatatagacgcatattatttagaaatgaaagtgctttgaatgatttagcttattttcagtttttttttttctgcagttgtaatcatatgacctgtttttctttctttttttttttttgagacagagtcttgctctgtcaccccggctggagtacaatggggcggtctcagctcactgcaacctccacctcccaggttcaggcgattcttctgcctcagcctccctagtagctgggactacaggcgcatgccaccacacctggctaatttttttattcttagtagagatggggtttcactgtgttagccaggatggtctcgaactcctgaccttgtgatctgcccacctctgcctcccaaagtgctgggattacaggcatgagccactgcgcccggcccatatgacctgtttttcttttatagatgggggagaaatatgggaagtgacttggtgtcagtcatctgtgttggttaaatcaagaatataatccgtgttttgcttctgaatagctctttataacagtgattggttactttgggagtaaagattattatttagagacagagtcttgctttgtcgcccaggctagactgcagtggaatgatcgtagcctactgcagcctcagactcctggactctggtgatcctgcctcagcctcctgagtagctaggactagaggtgcatgccacatgcctggctataattattattaatttacgtttagcattagtttttttcttccagtaggctattttactttatttatttgattttgatgaagtttgattatttctagtttgcttccttctatgacccctacctgttgtgggtctccaggcaagcagtgcataggtagagccatccttaggtagcctttagacttaatattaggtgagctctccccacagatagcctctcctttatttgaatggaattatattttaagtttggaaatatttttcagcttatttagcctgttgaatttaataaaaataatatttaatcttttcagaggtcgaaacagtaacaaaggactgcctcagtctacggtgagtaactttaatgttacttattggggaaaattagtagctaaaacatgatctctaaccacagaccaaatgccaaggcaaaagattcccttcttttgaattttgtcatagataacttgactgtttaagtatgttattagcctatatgtgtttttttaatgactctgtataaaatgtacaattacttgttgtattagtccattcttacactgctaataaagatatacctaagactgggtaatttataaaggaaagaggtttaattgactcatgctctgcattgctggggaggcctcaggaaacttacaatcatggtggaaggggaagcaaacacatccttcttcacatagcgacaggagagagaagtgctgagcaaagcagggaaagccccttataaaaccatcagatctcctgagaactcactcactatcatgagagcagcgtaggggaaactgcccccatgattcagttatctccacctggtcttgcccttgacacacgagaattattataattaaagataagatttgggtggggacacagaaccaaaccatatcatttgtaaatagtatttttgtcacgtgtaataacaagaacaagtcgcttgttcttttctaaatgactaagtgcaaatctaagtgaaaaacctccaaaagatacgtagaacaccaagagtggagtctgcagagttctttatgctttttattttgaattaatgtgctttttttctgctgctttcatttttctcctttggctttctggtcttaaattttggaatgttatcaatgaaattgaaccggacatgaagggcagaaactataagtcccacatgatggaagaaataaatgagaagctatcacaaatttttgagactttgcctttattagattgttttacaagaatcaggaagatatacacgtatatggtagtaatatggagtagtgtggttgatcagacttaagcactgtcactgatgctgatatgctgggagaacctagtcagggttcttctatgaaggtatgacctggcttcctaccccatttatttatacttcacccttcttagggtacatttctgtgagttttaacaattgcatacaatcagtgtaactaccaccacaatcaagttaatagaacagtttcattgcccaccaaaatccctcaaatcacttttcagtgaaccctcctctctctccaaccattgatttgtcttccatccttacggtttgtgtccttcctcctctatggaagtttactcttgcttttttatgtcatgtttagtcaaaacaccattagttggtttgactgataacacttgaaaacctgaccttctgttccttctgttctctatggaagcaaaatattaaataaacaaaatcttcccttaatacatgtaagatatcataaacctaactaaacattttgcaacaaataataaacgttagctttatatgcaaatgtaaatacaggctgagcatccctaatcggaaatgctccaaaatttcatattttgaattagggatgttcaagcactaagtataatgcaaatatccccaaatccgaaaaaaatccgcagtctaaaatacttctggtcccaagcattttagatgaggaagattcagtttgtactaatttctaatagttttttttttttttaatattccagatttcttttgatggaatctatgcaaatatgaggatggttcatatacttacatcagttgttgtaagttattagattattggggataaactgccttgggggtagaataaagtaattccatgaagttaaaatgtggataaatgattgtcaaagtaacattgcttagatcatgtttagtcaggatgatttagagaaatagattagaactccttttatccagtctaatataattcattgtaaaagtacagttggtcctctgcatctgtgggttccatattcatggattcagccaaccttggatcaaaaatatttgttaaaaaggccaggcacagtgactcacgcctgtaatcccagcactttgggagtttgaggtgggcagatggcttgagctcacaagtttaagaccagcctgggcaacatggcagaactccgtctctacaaaaagtaaaaaaactagccgaacgtggtggtacgtgcctgtagtcctagtgacttgggaggctgacgtgggaggattgtttgagcctgggaggtggaggtttcactgagctgagataatgcccctgcactcagcctggtcaacagtgccagacagaccccttctcaaaaaaaaaaattttttttttttttttttttttttttttttttgagaaaaaagaggcatggttgcgtctgaaccaaagatgtacggacgtttttcttgtcattattcctaaaacaatacagtatgacaatttacatagcatttacattatattaggtattacaagtaatttagggatagtttaaagtatttgggagaatgtgcttagttatatgcaaatactattacattttatgtaagtgacttaagtattatgtaattcggtatctgaaggaggtcctggaaccagtcccctaccaataacaacagatagctgtattcttgttaaccctgctgtgtgtgtaaaataatgttagtagttgattgtcttttgtacattattttgtcacttaaaatagctggggtcagaaatgtttgacttcagtattaaaattcgtactgcaaactctgagtagagcctcctgaagaatttcaagagttcagtgtattgttaatgttttgaaatttttttattgttttgttagtgaatacctaatattgaatgaagcctgatgaggtataaaaagtaaaatgaaaacaaatatccctggtgaccgggtagtatactgtttctttgataaataaattatatgtttttagggctccaaatgtgaagtacaagtgaaaaatggaggtatatatgaaggagtttttaaaacttacagtccgaaggtaatttttactttttttctttttcttacaaagtaaaagaacattttcatagtcagtgttttacctagtttttaaagccactttgaatgattttacttctcagtttcaaatactgattattttatagactggtttgtgtaatcagagaggcttcttgatgtgtgtgcttattaaaatatttcaaccatttttaagcattgtgagctaatagagggatgtggtggtttgttttttcctcttaaaaattattattaatgtacttaagacaaaccatagaaacaaaaaacatttagatatgaggatttttaaatgatggaatggataatagatcatatgcctgggaaaaagggtatgattctcttgagattatttttgtcaaaggcatataagaactggtaccttgatgagctaaagaattcctaacaaattttattttgtaaaggtttggagtacttacttgtgtttttcattttagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagaggtgggttttgatttcctaaatatgcctcatggtttattagatttattcaagcaaagattttcacagtgatcttacaaactttttttaaagaaatatctgggctgggtatggcggctcattcctgtaatcttagcacttagggaggctgaggcgggtggatcacctgaggtcaggagttcgagaccagcctggccaacatggcgaaaccccgtctctactaaaaatacaaaaatttatttttgtgtgtggtggcgtgcgcctatagtcctagctactagggaggctgagacagaattgcttgaacccaggaggcagaggttgcagtgagctgataccgcaccactgcactccagcctgggtgacagagcaagactccgtttcaaaaaaaaaaagaaagaaaaaagaaatatctactttctagaatagcccaagtaaggtaattttttagaaaaatgagaatgttaatgcatttttgttggaaaacaattagaactttagagaaaaattaaatagagtttttgtgatctcttaaaaaattagtttgtaaagcattttctacagttttgtggtcaagaatgctactgattatattcaactgaaaatttcttgtcccatttggcctacaatgctttagtttataagtgggcatgtggcaaatctggaaagaaatcaaagtataaggctaaggaagaaaggtagagaacggttggtagaaaacaattgtctaatgaaaatgaaaaagggtgaagaagtagaacatacgtattttaaaaatattcagagtatgagacaaggttttgagaatttaaaagcgattatgtagttatattaaaaatttagtctctttttaagtgtccattgatgaacaaagtgggaattcctgttactcatttgcaaggcattattgagtgttcagtaacacgttgcaaggcacttctgggcaatcctgaacttggttctcaaattctttttttttttttttttgagacggagtcttgttctgtcccctgggtggagtgcagtggcacgatctcggctcactgcagcctctgcctcccaggttcaagcgattctcctgcctcagcctcctgagtagctgggactacaggcgtgtgccaccacaccaagctaatttttgtattttttgtagagacagggtttcaccatgttggccaggatggtctcgattgtttgacctcgtgatccgcccgcctcggcctcccaaagtgctggaattacaggcatgagccactgcacccagccggttctaaaattcttttatttatttgtatatgccaaattctgtagtgaaatacgtaattctgttgtaaattgtagttcagtacaatttgattttcactattcaaatctataccaaaagctgtttttattgttgggctgattcttctacactgttacttggaaataataatataccaggattctttctcttagacttaggagtctttctctttgcttgctttttcagaggctaacagtactgggtattctttaactgtcttgatatgctgatgaaagcacagtgttctgtttttgaatcttctcaaatgtccttgtctttgattcacaactttttgtcttaagaggccttcagcatcccatacaaggaaacaagtctttttttagctgctacctttggagttgattttgtttatgtctaggagcactaaattatttatacttatactattgaaatattcctctgttataaattcaaaaattgactttggaagataaaattttagttgaatttaatacatagcactctggaaagagtattggccacaacaaaaaaaaaggttccctactctattggataccaggtcatttaacagccatttacggtatgcattgtctttttgtttttatgatgaattgatatttcccaaatgtggaagagtgaatattactttgagatgtttgtgatagtccattccttgctcctcttcaaaattaatgtcattaaatttttattactttattagatcttcatttctcagataattttagttcattatagaaaggcaagaaaatacagatcagagtgacaactttgaaaatctcactctactcataaggggatgggtgtattttgctatatattacaaaattagttttcttgatgaggacatccactattggagtaatttcaggtatcttattttttcttttctctctctttttttttttttttttggagacggagtttcgctctgttgcccaggctggagtgcagtggcctgatctcggctcaccgcaacctctgcctcctgggttcaagcgattctcttgcctcagcctcccgagtagctggttactgaggcatgtgccaccatgcccggctaatttttgtatttttagtagagacggggtttcactatgttggccaggctggtcttgaactcctgaccttgtgatcctcctgccttggcctcccagagtgctgggattataggcgtgagccaccacgcctgggcaggtatcttatttcaaaacttacagtggtttagtgaattatacaattgcgtccagtgcgtagtatcctgaaaatagtattaagtcatgtgtttaggacatcaggtctcttaagctaagactatccaggcagaaattgccctcttctataaaagaagaaaagtattaattaggaagtactatcagtatggagaaaaccattttagaattattaattggcatggtttccttctttttttttttatttcgagatggagtctcactctatttcccaggctggagtgcagtggtgcgatctcggctcactgcaacctctgcctcctgggtttaagcgattctcctgcctcagcctcccgagtagctgggattataggcacataccaccatgccctgctaatttttttttttgtttgtattcttagtacagactgggtttcaccatgttggccaggccgcatggttttccttaataacaaaattaaggcatttattactgcatctagattttttttattttttattagagacttactcagattactcccaaagtaaaggaaggtatggtttaatcaatgcttcttaatgctgggttcacgtttagtcacctggggagtttttaaaaatgttctcacttctagggatcctggtttaattataattagcctgggtgaggctctggacagtcagggtgtgagctatgggtttcatgtgatgagatcccaggagtggctctgttctgtggccttgagaatttgtgctttctaggccaggtgcggtggctcactcctgtaatctcactttgggagaccaaggtgggcagatcatttgaggtcaggagttcgagaccagcctggccaacatgttgaaaccccgtctttactaaaaaagtaaaaaattagcgggacgtgatggcacatgtctataatcccagctacttggggagaggctgaggcagaagaatcgcttgaacccgggaggcagagattgcgagatcatgccactgcactccagcctgggcaacagaataaaaaaagaatttgtgctttattttcttgcctcacagtcccctttctgtctcagaattggcaactgcctgaaatagtctctgctgttatcatttgatagtacttttccacatcttgaatggatagatagagtgttttttataatagaagtggatgaatgattagagtatactaatatgacattgtattttcctaaaagatatgaattgatttcatttctgagcttttataattctcttctgtaatagtctgtcaaattattaaggttgataatattaactaaaatttgagtgcatattctatgtgccagactctgtgctaacagatttacctacatttgttcacataatcatcacaagttgtttctgtagtagatacagctattatccacgtcatagatgaggaaacaggcatatttaggaaacttgctaaagtgaggacacaaatctagcttttctactctaactcatgttcttaacattatactgcagtgacataaattatgtggtttggtttgttgtttatctcagttgtcataagtcgaattaatgtttgtttgtttgttttgagacagagtcttgctctgtcgcccaggctgggtacagtggcgtgatcttggcgcactgcaacctccacctcctgggttcaagcagttatcttgcttcagcctccctaataactgggattacaggcacgtaccaccacacccgggtaatttttgtatttttagtagagatggggttttaccatgttggccaggctgatttcaagctcctgaccttaggtgatccacccacctgggcctcccaaattgctgggattgtaggcatgaaccactgtgcccagccagtaagttccatggttgttaaaggatttctccacaaataaagctaaaagtaaaaaaaaaaaaaaaaaaaaaaaattctcaagcaatataagatgcagactattatgttgttcaagttttttttttttttttttaatctttggctttatttttggggaaaccttttttttcttttttgttttccttgggacggagttttgctcttgtcgcccaggctggagtgcaatggtgcaatcttcgctcactgcaacctccgccttctgggttcaagcgattctcctatctcagcctcccgagtagctgggattacaggcatgtgccaccatgcccggctaactttgtatttttagtagagactgggtttctccacgttggtcaggctggtcttgaactcctgacctcaggtgatccacctgcctaggcctcccaaagtgctgggatcacaagcgtgagccaccgcgcccagccagggaaacctttattttgaggcggagtctcgctctgtcacccaggctggagtgcagtggcgtgatctcagctcactgcaacctctgcttcctaggttcaagcaattcttctgcctaagcctcccgaggagctgggattataggcgtctgccaccatgcccagctaatttttatatttttagtagagacggggtttcaccatattggccaggctcttctcaaattcctgacctcatgatccacccaccttggcctcccaaagtgctaggattacaggcgtgagccaccacactcggctgctggggaaaccttttaacatgagtaaggtcagtgtgacttttaagttcttgatgctaacatcattgatttcaataaagtttaaaagttatattcatgcatatatgcaaatgaataaaaggctttgaaatagtgacttcttacggtacagtgaataagtttcctttggtctcttgaatgttatacatgttccagtttgatttactgagaaactgaaagtacctttacgtcatatgagctgtgagtcaccttggcacattcataattagaagagaccatcagattatcattggaaaatcagtttgtatttatcctttatttgaattccagtgcagacagatctgaggttctcttcattttgctaaaacttcttagggccttcagtcgcttttggctctgtattcgtgtatctttggaattgtcctgttatctctgcttgttttttacttgattttccatccatttccagtattcctttctcctctatttttttccttcattttctttctgctcttcctgttgcgccattattcatgttttcctctttactccaactcaactatggctttacttctgtttccttattccattgttcctcatactttttcctactgcttcattttctttgcagtattctcagcctagatgataggggtcagcaaatctgctcatcagtaaataaattttattgtagcatagctatgcccatgcgtttgtgcattgtctatggctgttttgatggctgtagccatagagttgagtagttgtagctgactgtaggacttgcaaagccagaaaatttgactgtctctttacagaaaagtttgccagctcttggcctaaatcatattttccgctgcatttagggctttttaggactgatcaaaaatacatgctatactggctttggtgaagtaacagaatgtgctctgtcctttaaacttacaactaattgcatgctttgattctaatactgtataatatcctgcgattcttattcatgaccattctaattggatttagtctgaagaattacttttgcttaacagattctttgtcacatttagtgaaaaatcataaaaggggaaggttggttaatggaaaagatctccatcaactaaccactaccttccttatctacaaatttatcttcttcctccgtgccatctttttttttttttttttcagatgatcttgctctgttgcccaggctggagtgcagtgatgcaatcacagctcactgcagcctcgacttcccaggctcaggtgatcctctcacctcaacctcctacataactgggactgtatgtgcacatcactatgcctgactaattttttatatttatattttttgtagagatggggtttccctgtattgcacaggctggtctcaaactgctgggcctaagagtcttcccaccttggcctcccaaagtcctgggattacatgagtcaccgcacccggcctcattattatttttcctctggttttagtagagaggatttttaagccaacttcaatcatgcccttgactctctcccttctacttacctccttgttctctttttctttttctttttttttagatggagtctcggtctgtcacccaggctgaagtgcagtggcgtgatttcagctcactgcagcctcagcctcctgagtagctggggctataggtgcctgccaccacgcccggctaatttttgtatttttagtagagatggggtttcaccatgttggccaggctggtctcgaactcctgacctcaagtgatcacctgcctcagcctcccaaagtgctgggattacaggcgtgagccaccacgcctggccatctttttttttctccttgctcttttataccacttctctgtttctgggctcttcaacatctgcctttctagttaatctttccctttagcatgaaaacctattcacttcctgctcatcctaaaaaggattcttttttgttttgttttgtttttgtttttgagacagagtctcgctcttgcccaggctggagtgcagtggcactatcttggctcactgcaagctccgcctcccgggttcacgccattctcctgcctcagcctcccgagtagctgggactacaggcacctgccaccacgcccagctaaatttttgtatttttagtagagatggggtttcaccgtgttagctaggatggtctcgatctcctgaccttgtgatccatctgcctcggcctcccaaagtgctgggattacaggcatgagccaccgcactgggcccaaaaggattctttttaatcctgaattcttctagccattatcctgcctaaggctacgattaacctctaactgccaggtcctttggaatctttttctgtctttattgctgcacttgaatgttggtttcaccctccttcagaatttcctcttctgtattttttatgtttattgatcattccttccctgcctcattcctgggcttcttttccttcacacaccccttagatgtgtgtccccagtgtttgtttctttgcctgctgctcttgccacatgacacacactgccagctaccacacacaagttccctcctatcatgtgtgtatcattgcccttataccatgttgtattaaaattatatgcttgtctcccctgttacagtttgagctctttgtgctccaagtaaagacagtgatactgtctttattatttattctcatggtctagtatagtgctttggcacatagtacaggctcaatataaatgtgtttgaataaatgaaattcagtgccttaatacacttttgtagaagcattattttatggaaagaatgaaaaagctgtaagtggtcttacatatatagtcatccagcagatacttagagagctctgggatgtgttccttgctgtgcttgttgctatggacagtacggagaaatacaagaatctattttgggtcccttttgagaacctagtgaaactgtgtacctagtgaaactgtataccctcaccctagaaaaatttacacacatgtagattttacatgtaattcttttaaaaattaattttttttcttttttttaaagaaacagggtcatgctctgtcactcaggctggaatgcagtggtgtgatcatggcttactgtagcctcgacctcctggctcaagcgactctcccacctcagcctcccaagtagctggggctacaggtgcacgccgctatgcccggctaatttttaaaaatattttatagacactggttctcactatgtttcccaggctggcctttacctcctgggttcaagcaatcctctaccttggccttcaaaagtgatgggattataggtgcaagccactgtgcccacgctaatgtaatttcatggtgttcacagtttcttcagggagttcatatacgccatgtactctattctaagcatttttagagttagagatagcaaagcacgtgaataaattcaagaaaaatggaatgttgtactgcatgacattgaatatcaaatggagtcagcgatgcaaataattgtctagattttacaaaaaaaattagcctggtgtgctggtgtgcgcctctaatcccagctactcgggaggctgagacaggagaatcatttgaacccagaaggtggaggttgcaatgagctgagatcgtaccactgcactccagcctgagtgacagagcgagactccatctcaaaaataaaaaataaaagaattgtgtagattttagtagttggaagaagttggagtgttaatgtgtaattagagaacagtgagaaataaaattctacagattgttttattctggtgtgctgttgtgttctcatatggttgtctttttggtcttgatagtgtatcagtaacagagtacgagtaacaaacagggatctcttctgaacggcgtgacattagaaaagctgtttacggcctcaactttgctgtggtttattaagacacagatatgtgttcattctggggccaagcagtaactggagagtggcacttattgaggccagtatggaggcagtacagagattattgagattaaaagaaagaaacaggtggaacggatctatgtaatggaaagctaaacagaatagttcgtggtacacagtagaaaagcattacatgtttattaagatatggtcatcttccatttattaaagttacatgttttataatttttagagtatatagaaattctctaccctatcatgtttgccaaagtcagaacaataacttcatttattaaatataaaaaaaataaaaacctctagcataaaatagaattttatttggacaaacgataaaaaaatactgtgtggtactagtaagagtaaggttgattcaagatacatgggagcagaatccaaagtgtagaaataggccaggtgcagtggctcatgcctgtaatttcaacacttttggaggctgaggcgggaggatgagttcaggagttcaagactcgccttggcaacttggcaaaaccccatctctacaaaaagtacaaaaattagccgggtgtggtggtgtactcctgtaaacccagctacttggtgggctgaggtgagaggttcacttgcagccagtaagtcaaggctgcagtgagctgtggttatgccacggcactccagctgggtgacaagcaagaccttgtctcaaaaacaaaccagccaggcgtggcggatcacctgaggtaaggagttggagaccagcctggccgacatggctctactaaaaatacaaaaattagctgggcgaggtgacgggcacctgtaatcccagctacttgggaggctgaggcaggagaatcgcttgaatccaggagacggagtttgcaatgagccgagatggtggtgctgcactccagcctgggtgacagagccagactctgtctcaaaaacaaaaataagcataggacatggggataaattgaagatttatgaagacacagctgaaggagacataaaagtagatttggctaaatggaaacatgccatactttgaatggaattatttaatactacaacgttgtcaattttcctcaaataaatctctaaagataatatattcagttttggccgggcacgttggctcacgcctgtaatcccagcactttggaaggctgaggtgggccgatcacttgaggacgggagtttgagaccagcctggccaacatggtgaaaccctgtctctactaaaaatacaaaaatcatctggacatggtggcaggtaccagctacttgggaagctgaggcaggagaattactcgaaccccgtaggtggaggttgcagtgagctgagattgcactccagccgggtgactccatctcaaaaaaaaaaaaatttttataatatatatatatatatccgtttttgtagaaattgacaaaatgattctaaagcttattagattatgtgtattaacagaagaactttggaaatttttttccacaagagtcataaaggaggacttgccctacaaaatatgtcagaattaaaacataacttgtcagctgggtgcggtggctcacgcctataattccagcactttgggaggctgaggcaggcagatcatgaccagcctgaccaacatggagaaaccccgtctctactaaaaatacaaaattagccggtcatggtggcgcatacctgtagtcccagctactcgggaggctgaggcaggagaatcgcttgaactcgggaggtggaggttgcagtgagccgagatcgcgccattgcactccagcctgggcaacaagagtaaaactctgtttcaaaaaaaaaaaaaaaaaaaaaagaattataactgtcacagtggctacgtatggagcatccaaaactgaatttatgtgggtattttattaatatgcaatatagcactttaattctggaggaaaggtggattattcagtaaatgattctgggacattggggacaaattagatacctacttcacactgataaataaaaccaaatagattaatgagaaaactgtgattaaacaaaacaacacccagactacactggagcaaatctgtgaatttgtttaattttgagtggagaaggactttataagcatgactaccagagcaaaaaaatcatgaagtaaaagatcgatacctttgattataaagagattaaagatttaggccgggtgtggtgctcacgcttgtaatcccagcactttgggaggccaaagcgggtggatcacttgaggtcaggagtttgagaccaacctggtcaacctggtgaaaccccatctctactaaaaatacaaaaaaattagtcaggcatggtagcacatgcctgtaatcccagctactcaggaggctaaggcaggagaattgcttgaatttgggaagtggaggttgcagtgagccgagattgtgccacatcactccagcttgggcgacagagtgactccatctcaaaaaaaaaaaaaaaaaagacttagacgtgtccaaaagtaccatacatttaaaaagacatgccacaaactgggaaaagtagaaaaatagttttaaaaatgaccagtgaatgtatgaaaaggtggccctcctcacttgtaatgatttaagaaatgcagtttatttttattttattgtatttttaaagaaattcagttttaaagcagtggaatatgattgtctatcagcttgcgctgaatggtaaatgtgagaaagattactactacttagtggtactgagggagttgcaaaacacttaacactgctagtgggatggtttaagtaaaacaagtagcattcttaaactctctattaggtaaagaataggtaagtaatgcatatgtttccaggacattttcagtaagactgtttactgatagggttgtgtaatgctaatatacttactatctagttttagtattatttttttctcttgtcttggatggtttcaatggagtcttatgcatgcagatatattaaaactagtaataaagcaagagaaggaatgtggataaattatctctaatttctattttgttctatttctatttcatactcctgggaaagaatattaagtgggcatgtgtacttgaacagttgttctgttttttattagaaaagaatccgaatctataaaatgttttacatatttgccagggaaacagaaaagatatttgtacagctgtaagaattggaattaatttcattttactgacttttccttaacctaattctgaacacttttgccataggtttgagaataagttgttataaaatgactactattcttcactaatagtattggcatttcaattcctaaattctgttttttgattcttgaacatttctgaatttactttttttgtcttagttcttctacagaatcattttcttcttttttctttttttatttttattttttatttttgagacagagtcttgctctgttgcccaggctggagtgcagtagcgcgatctcggctcactgcaagctccgcctcccgggttcatgccattttctcctgcctcagcctcccgggtagctgggactagaggtacccgccacagcgcccggctaattttttgtatttttagtagagacggggtttcaccgtgttagccaaggtggtctcaatctcctgacctcgtgatccatccgcctcggcctcccaaagtgctgggattacaggcatgagccatcgcacccggccttctttttttctttctctttaacttctgagctgaaaatagtaccttttataaagaagtgctcaaacgatgattggactgatttctccttatttctctctttctctctgtctctttcactctctttttagaatttttcttttttaagtagagacgaggtcccactatgttgcccaggctgtcttgaactcctgagcccaagcaatcctctttgcctcagcctcccaaagtgctcggattacaggcttaagctatcacaccaggcctaggctaatttcatattttgagatggcacaaatttctttcaggtagctagcttttcctcctcctccccacttaaaatagatcctgatccagaagcctaatggagaaaatgaaaacagaatgttcacccataaacagtatctttgtattggaatcttttctaaaacttcttttgatctttttaggagatagtgtgggaatcagcaatctagtattacgtacgtggaatctgtcaccttgtttttttaaatacagcaaacctcatgaagtgaatttccatattttttcttgttcttgttagttttgcaccactcaggctttgctgtagaatttgatgtatatttgattctgtagagcatgggctattgatcttcactcagctttcagaggaatctgattagtaagtttgagttttttattattttttagttgattttgaagtaaaatacagcaccattttaactgataccatttctaaacaattttcagttcaaattttaagttagctaatttagagcttaagaaaattgctttaaaaacataaaattactggctgggtacagtggctcattcctgtaatctcagcactttgggaggccaaggcagatgaattgcttgagcccagtagttcaagaccagcctgggcaatatggtggaaccccgtttctacaaaaaaaatacaaaaagtagccagacacggtggtatgtacctgtagtcccagctattcgggtggcagaggtgagaggatcatctgagcgcagggagattgaggctgcagtgagccaagtgagaccctggtttcaaaaaaaaaaaggttactaattgcagtgccttttatcttatttaatgggcttagtcaaactaagatgatgtattttatcttataaatgttttcccttgaattttaactgaagaatccaatttgtacctctcacaaacagaatgtattagtaaggaaaataaatactgctttttattacttaaataggatatatttttctcttagggattttttttctattttatctcactttatcgtagtgctagaaaatttaatcattcatttgagatagggagaaaattaggtttttttttttcttctattttgagacagggtctcattttgttgtccaggctggagtgcagtggcgccatcgtagctcaccataacctcaaactcatgggttcaggtgattcaccttagcctcctgattaagctgggactgcagatgtgtatcaccactcctggctaatttttgttgttattttttgtttgatgaggtctcattatgttgcccaggctggtctcaaactctgggcctcaaatgatcctcctgccccagcctcccaaagtgctgggattacaggcatgaacctctgctcccagcccattttttaaaatatattcacagcattgtgcaaccatcactacaatcaatttacattttcatcaccctgaaaagaaactctgaaccccttagcagttcctctctgtttgtttcaattttccccagctccaggcaactattgatttattgtcttcataggtttgcccattctggacattgcgtattaatggaatcatataatatatagcctttttttttcttttttttttttgaaacagagtctcactgtgtcgcccaggctggagcgcagtggcatgattgcagctcactgcatcctctgcctcccaggttgaagcgattctcctgcctcagcctcttgagtagctgggactataggcgcctgccaccacacctactaattttatatttttagtaaagacggggttgcaccatgttggccaggctggtctcgaattcctgacctcaagtgatctgcccacctcggactcccaaagtgctgggattgcagccatgagccaccgcatctggccatatatattatgataggcttgtttcacttagtatgtttcttccatgctgtagcatgtattagtacttctttctttttcatggccaaatattccattatacagttacacaggtacactacattttgtttattcatcagttggtggacattttcattgtttccaccttttgatttatacataatcctgctgcgaacagtgacttttaaagtttttgtgtgggccgggtgtggtggctcatgcctctgtaatcccagcactttgggaggctggggctggcagatcatttgaggccgggagttcgagaccagcctgcccaacatggtgaaaccctgtctctactaaaaatacaaaaatgagctgggtgtggtggcgtgcacctgtaatctcagctactagggaggctgaggcagagaatcacttgaagctgggaagccgaggctacagtgagccgagatcacgccactgcactccagcctgggtgacagagtgaaacttcatctcaaaaaaaaaaaaaaaaaaaaaaaactgcgtgtggacataggttttcaattctcatgggggtgtgtgtgtatgcatactcatacatacatacacatacctgcaagataattgctggctcgtatgctaaatctatgttgaaccttttacataactgttgggctgttttgttttctttttattattttttgaaaatagagttggggtctcactgttgcacaggctgatttcctgggcatagtggctgtatcattttacaatcctacatagctgtttccaacgtagctgtatcattttacaatcctactagcagtgtctgaggtttcttatgtttttcacatcctcaccagcatttgttattgtctgtctctttgattatacccatcctagtgggagagtaagaagtagtatctcactgtagattttttttttctgtttacaactttactttaaaaattatatatgcacacatggtaaaaagttcaaaacgtgtgtaccaaaagatttaacagtgaaaatagaaaataagtgtggtccttgttttcttccaccaaggcaaatattgttataatctcctaaacaacttgtcttccagatttctcattttcagtcaatcttgggcattgacataaagaaattcttagacattgcttttattagatcatctcatcccttgctcaaaatcttcagtggccactgttgtttacagaataaagttgggatgctatacagggcccttcccagtggaacttctctttttcaaccttatctctcattatttcccaatgtttttttttttttttttgagacggagtctcgctctgtcgcccaggctggagtgcagtggcgggatctcggctcactgcaagctccgcctcctgggttcacgccattctcctgcctcagcctcccaagtagctgggactacaggcgcccgccactacgcccggctaattttttgtatttttagtagagacggggtttcaccgttttagccgggatggtctcgatctcctgacctcgtgatccgcccacctcggcctcccaaagtgctgggattacaggcgtgagccaccgcgcccggcctatttcccaatgttaatctacttattgacctactaagctggcatgttctgtgtgttagacatcaccaactttgtgccttctttttttgtttgtttttgagttggagtctcactctgttgcccaggttggagtgcagtggcgcgatcttggctcaccacaacctctgcctcccgggttccagtgattctcctgcctgagcctcccgagaagctgagacgacaggcgcgcgccaccatgccctgctaacttttgtatttttagtagagatgggtttcactgtgtttcccaggctggtctcgaactcctgaccttgtgatccacctgccttgggctcccaaattgctgggattacaggcgtgagccaccgcggccccctgtgccttcttcttttactcctggatttaatcccaacgtgaagaatctaccttactaactagagttttagatactttttcaaaaccaagcccacatctgtcctttttagagtcttctctgaccttccctgctcattgtggtttgtttttattgcctgtaacaatggctgttaaactttacattttaaattaatttatgtttgtatgtatttatttgttgagaaagggtctctctctgtcacccctactagaatgcagtggcgccatcatggcttactgcttcctgggctcaagctgttctcccatttcagcctccccatgcaccaccctacctggctaatttttttgtttgttttttttagtttagtttttgtagagacagatgtctcactgtgttgcacaggctgatcttgaactcctgggctcacttgatcctcccatctcagcctccccaagtgctgggattacaggtgtgagtcaccatgcccagactttaacattttctttttagtatagaataggtcagtttttttccctctgatgagatcccatgctgactcttagttaaaacaaggctttggttggaagaagagctagtgatgtcctagctccctacttactccactttcccttgccttctggggtgtcctgaagacatcatagggtgtcatgaagtacagttggagaaccagtggtctccatcatgtaccaaacactcatcttcacgaagcagtatgtagtgtcttttttaccggtatattttctctctcccaatgcattaaacttttctggagttcagaaaacaaatttatagaattaaggaaatgcgtcccccccaaccatggtgtctagtatatatacagtgacttacagataacaggtgttcaacatatatatattcctttgattgatttttgaaaagtttacatgtatatattttttatatacggggtctcactctatcactgaggttggagtgtggtgatgcagatcttggctcaccgcaacctcctcctcccaggctcaagtgattctcccacctcagcctcccgagtacctgggaccacaggtgcgcatcaccatgcctggctaattttttatatttttggtagagacaggattttgccgtgttgcccaggttggtttcgaactcctgagctcaggcagtccacctgccttggcttcccaagtgtgagccaccactgaaatacttatatttttaaacttaatttatttatatttattatatttttatgtttttatattttaaaaaatatttttatactcactagacccaattttatactcctaaaccagggaataactgtttttttttctcttacataggcatgataccatagacaatgattaaaattgtaattaccattcatttcttagttttgtggctgggacactgatgtcttcaaatgttagtttgcaaatacagtcagccctctctatccatgggttacacagctgtgaattcaaccaaccatggatccaaaatatatgggaaatacgctggggctgtgggtcacacctgtaattccagcacttagggaggctgaggcagatggatcacctgaggtcaggagttcaagaccagcctggccaacatggcaaaaccctagctctactataagtacaaaaaattagctggccatggtagtgcacatgtgtaatcccagctactcgagaggttgagacaagcaatttgcttgaacctgagaagtagaggtttccatgagctgagattgtgtcactgcactccagcctgcgcaacagagtgtgagaagaaaagaaaaaaaactgtctgaaaagaaaaaaaaaaattatatgggaaatcaaaagcatctatactgaacatgtacagactttttttcttgtcattattccttaagcagtaccacaactatttccgtagcatttactttgtattaggtattataggtaacctagaggtttaaagtatgcgagagtatgcaaatactacaccactttgtatcagggacttaagcatccctggattttggtatccctagggggtattagaaccaatcccccatagatgctgaaggacaactgtagtgtgtgttggaataatttattttcaaatggatcatttggagaacactattctttaggaaacatagcctcctaagttctgttccatacatccctttcacctccacggcgttgtagcatcctgctttcatgactgtgtcatcactcggaaggaactgcttctcttccagaatgcttttcaagatctactctgaccacagctataaactttacacttctattctcttcttgcccctcacagtgttctctgttcctctaagatcttaaactctgtctactcctaatccagcctgctgggtgtggctggagaaagtcccactggggggctgattagttaggaatgtagggtttccagctcttgctggagcctcagaagagttcagcagacttttttttttttttttttccttaaacctatttctgcagccttgatgaccactccttccagtccctcacctatttgctttattcatggcagaggctctttcttcctgcttgtcagtacaaagaggcaggattcttcacctggatctgtggattctcaaagaatttgtggagagaattcagggcattgatgaccttggatgaagagaaatttacatctttatttacactaaccttcaagtgaaatttagcattttttgccatttaaaaatatgggcaacaaacaactagtagtattagcagtatttatgacttaagcacctatagaactcagttaatttcatatcgcttgatgttatgggtatctcaaattattattttatgtatatatatttttgagatggagtctcgctctgtctcccaggctgagtgcagtggtgcagtctcagcccattgcaacctctgcctcctgggttcaaacgattctcctgcctcagcctcctgagtagctgggattacaggcgcacaccaccacgcctagctaatgtttgtattttcagtagagaaggggtttcaccatattggccaggctggtcaccaactcctgacctcaagtgatccgcctgccttggcttccaaagtgctgggattacaggtgtgagccaccgcacccggcctcaaattatttttagaaacagaatcttgatatggtatccgctctggccttgaacttgtgggctcaggcagtcctcccacctcagcctcctgagtagctgggattataggcatgtgccactgcaccaggcttcaaattattatgtatgttcatcacctctttaaatttataatagttattaaacctgttactggatcttaatatttaatgctttaattaagaacatgtatgttactatgccaacagatttttttagtttttgataactgcatttcattgttacttgttctcatttgatttcctgtgtattttacgaatttaagtacattctgaatacggtttcataggcttccctaaaatattgaaggggcccatggattaagaaaaaggctaagaatccctaatctagaggctccccacagtcctcttttgtcatcatacccctaccccattctagcctgaggagcgtggctccacctgtgcccttggttttgttgttccagtccatacatcctgcacccttaactgtgtttcttatccccaacttgtttctttgtgttattcttcagtattatagtctttaatataatctgtataatacatggtgtagtagtatatgctcgtagtatacaattcagttagaacagatgagtattcaatgaaaagataatctcctctctaacccccagtcccacttccctggggaagcctgtgttcttgtgtacaattcagaaaatgtttatacacatattttttatttatttattttttgagacggagtctcgctctcgccaggttggagtgcagtggcgcaatcttggctcactacaacctccgcctccctagtagttcaagcaattcaaggttcaagcaattcgcctgcctcagcctcccgagtagctgggactataggcgtgtaccaccacgcctacctaatttttgtatttttagtagagacagggtttcaccatgttggccaggatggtctcgatctcttgacctcatgatccacccgcctcagcctcccaaagtgctgggattacagatgtgagccactgtgcccagcctgttgatttaattttaaacagagtttcgctcttgttacccaggctggagtgcaatggtgcgatctcggctcaccgcagcctctgcctcccaggttcaagtgattctcctgcttcagcctcccgagcagctgggattacaggcatgcaccaccatgcacagctatatttagtagagatgggggtttctccatgttggtcaggctggtctcgaactccggacctcaggtgatccgcccgcctcggcctcccaaagtgatgggattacaggcgtcagccactgcaccccgcctatacacatttttttgttttttgtttttttgagatggagtctcgctctgttgtccaggctggagtgcagtggcgcgatctctgctcactgcaagctctgcctccctggttcacaccattctcctgcctcagcctcccgagtagctgggattacaggcgccggccactacgcccatctaactttttgtatttttagtagagatggggtttcaccgtgttaaccaggatggtcttgatctcctgacctcgtgatctgcctgactgggcctcccaaaatgctgagattacaggcgtgagccaccgctcccagctatacacgtatttttaatgccactccagtctatgttggaaccattttacttcccctttcttattttcttcttgtgttcttgaaggcctagatcagctgttgctgataggctgtcactgtcactttagaaagcccagagccttttgttccttagaactttgtttttaattgtattgtagcactcattgtattcgattctaaaagatttgcttcatttctgtaactagtctcttacacccaggagctcctagttcctacaggaaatgctgggaattgtatcagtcaaatgtgaatccccacctcgtccagacttatgagtgcattgtaggtactcagtaagtgctaaaaatgactaaatagtcccactgataccaatctatatactgatactttatatagtatatagattggtccacatataacgatgacacataatgagaaactgtcttaaaaagttgttgaaagtgccgcaggaataggaattgatcaaaacaatatgattttttaggtttatatggaactttgatgtttgagaaaaggctgatttagttgagaagaaatggttagctgaggattttgatgacttctctggaagcacatttgagggtttgtgatgttaaatctgatgttaatgattatttcatccagttttatgtcattttatagtttttatacatttaagtatatttatttctaatgtttaacactaccattttagttatttgaccattattctggccctttaaaaaatgctcagacaagtttgaatgatttttcagaggcattattggctcagaggtaaaagaggaaagattgagaagctgaatatgtactctgtttcctgggtatggggctggggatacccagaagaggttcacacgttggtcgagacatttctttatgaccaccagcaggtggcatcaccggcccaaaatgactaagtttctgcccagaatcagaagagaaggtgttgagagcccactgctgtgggggtagcatggaggtgggatacaggggctggaggtgatacaattttgtttcttcctccaacatcgcctgctagtctagaggcttttataaattgaaaaactaattctttatcatctcatctgatggtttttatgtttttccttttttctctctatacctgtagttccttcagaaacaggtaacacttttctaatagtcacgttgtattcttgcatcttgttgttacaatgcttttgtttctcaccataggggatgatggaaaattaatattctttgacttatggcattggtaaaatctgcatgcaaattcccacagttgcctgtagattagagccagttgtttttttctcaactttgcaggaatcctggttacaacattgtactatttactaccaacagtgttttttttttttaaaatccagacttgctgggcatagtggctcatgcctgtaatctcagcgacttgggaggctgaggtgggaggattgcttgagcccagggctgcagtgattgcggcactacactccagcatgagtgacaaagaccccatctctgaaaaaacaaaaacaaaaacaaattttttttaaagaaacagaaacaaaaatccaaacttgtaaccactgtaaaacaaatcagaatttacgatagtggatattattaatagtgcagaatggatacccagatcttgcttcctttctagctaatgatgcaatgttggcctgaaatgcattacttatagccagggattttctcagcatcctgatgatatagcctcatttcgtgctaactctccacttctgcacatcttcccctaagtcctttactcatctttagaaagagctacttttggtgaaattttaaaaccaaggaatatcattctttatagaatcacacttctgtgttttccccttccccatttctgtctcgaaagcgacagactgctacataacctgtgaatacttttttttaaaaaaagtttggtattgtaaacagaagatttaagattaaaatgtagcattgagaaaaatagatttattaataatgccctcttaacacaacctaaattctggtcagtggaataaagcctgggtcctaaagttttagacgcttgcttgcttttccacactggctcttacttggggatccttttagaaatttgtttagaataatactgtaaaaacatatttaagctactttgtgtgtacatttgggatcttttggtttgaagacggcttgactcaagactttctaaatattttcacacacacacacataccctgtagtgagaaaaaaatccgtttatatggttctataaaaatctctagctgcttcgagctttaatttcttgaatcaaaagagtattgtttttaatactgagcttctatctaaataaatgctttatttacttaaatgtgtgcttttcaaaaactagtatgattaagacattaacaggatcttagacgtaaaggaacagtcctgttgcttcttccagaagataatatgactcgtttggaattttcctatagtgtagttttttgtctagtgttgtgagaattaaagggatttcaggatcttaaggtaggttattatttgatgttttcttggaacattttacattcttgaaaatacacatggctaaattaatttttgccagcaatccacataactttaagataatgtagagaagaacgtgattcaggttagtatcaaataaggtcagatttctagtgccatcagtagctttcagcaaagatgaggtgttggtaagatagcattagtctcttagaatctcttagagagattttccaaaattcagccatttctagtgaatgctccattccacccccagctgagtcctgctgctctggggaactccctcagcacactcttggctcttagaattgctagcaatgggagtagtgctgctggtggagctggcagctaagcccagaggtggattaatgcttttattccctgatgtacaggtacacacactcatacctacccacacctagtttgggataagaagaggttagaattagctaggcttgaagttccatgcttaaatttgctggctcagatttcttattttggcatcactttgcccattagggagacaatgacagttatagaagcattgccaaataaaaaatccatctggaataacctcttttgtaggagtattgtgtgtttagttgttgattcgtcccttcctcctcttagtggcaacttacagtactgggaaggaacagtggctgggagcttatattcctcagcagagccagatcagcagaagtattactccttagttcgtagtaggtggtaccctatgggtccagtcatttaaatgcaagcctgtatctacagagcgtttcctagtgccatcattgcccagtgggcctttatttagctgagtctaactcccaactagagaaaatttcctgtgccagacagcagtatggtcagctaacatgtggatgctacatttgctttcataagtcagtactcttcaataacattagtagaagagaagaggacacaaagtgagagtgtgttaataggaagtccaggtatgcctgctacctgaactttctgagacaggtaatactgtagggcctgaactttgtagcagagtggttatatatgaagaagtgggttctgggaggggttaaaccacttagaatggcttcatttactaatggcaagagtttgctgggatattgaccactgtacatagacatgaatatggaaagttaaaaacaaaatccacatatatttggctgcaagtactccgaaggtatatctaattagtgcatccattaaacaaaagagatattttaggccgggcatggttgctcacacctgtaatcccagcactttgggaggccaaggtgggtggatcacctgaggtcaggagttcgagaccagcctggccaacatggtgaaaccctgtctctgctaaaaatacaaacattagctgggcgtgttggtgggcgcctgtaatcttagctacttgggaggctgaggcaggagattcccttgaacctggaaggtggatgttgcagggagccgagatggtgtcactgcactccggtctgggtgaaagagcaagctccatctcaaaaaagaaaaaaaaaaaaagagatatttttgatggattgatagaaattttctttttctttttttttttgagacagggtctcactctgtcgccaggctggagcacagtggcgtgatctccattcattgcaacctccacctcccgggttcaaacgattctccttcctcagcctcccgagtagctgggactacaggcatgtgccaccatgcccaactaatttttgtatttttagtagagagagggtttcaccatgttggccaggatggtctcgatctcttaacctcatgatccacctgcctgggcctcccaaagtgctggtattacaggcatgagccaccacatctggccagaaattttcttggtcacttctgagacatgcagagtaattacctgtaatataatttaatgaattatgtcaatatattaaaatatgcttcatgtgggctgggcatggtggctcatgcctgtaatcccagcactttgggaggccaaggtgggggtatcactaggtcaggagatcaagaccagcctggctaacacggtgaaaccccgtctactaaaaatacaaaaaattatccgggcgtggtggtacacacctgtagtcccagctactcgggagactgaggcaggagaatcgcttgaacccgggaggcagaggttgcagtgagccgagatcacgccactgcattccagcctgggcaacagaacgagactctatctcaaaaaaaaaaaaaaaatgcttcgtgtggcttaaaattatatgaaaagaaaatacctttactgatagtcatctgtgattccatttgctaaattaaacgtgaaagcatacttttactgaatactatatattccgtatcagtttagatagcagtttatcttcacatacataagttttaagtttacctttattatagtgcattggtcttttgttttcatcaacctaaattatgttcaataaatgtttctgttagattttaagttaaacaattatgtgaaattcatttttcgtaattgttttttaacatatgtctttgttggtaattcacgtgtgtgagtgtaactgattgccagattatataaactttcaaccaaaaccattctttgcagatgcttttactgactctgctatcagtgctaaagtgaatggcgaacacaaagagaaggacctggagccctgggatgcaggtgaactcacagccaatgaggaacttgaggctttggaaaatgacgtagtaagtaacatctttgtaattattgctagactctggtcagtatgacatcctgtcacttggttgtaatttaaatgtgcttttgttgttgttgttattgtagtgagtgtatttagagcagcaggtttgttgtataactagagactttctcccaagcaatatataaagaaaaatgtttgtcattttacttgtaggggttaagcaggagtactgtctgttcttgtggatgctcatgaattacttctttgtgattaaaataaataataagaagtagcttaaattaaaattagaaaccatgggaaatgccggtgtgttttgctttaacacccagccaaataaggtagcctaaggaaagtggtgtcttaattgttgacttcacctagagaagaggttgaagtaggacattttaagcctcttgtctgaagaaaaggttgtcattaagataaataattaggttacattggaattaaagcattacataaatttcttggtcttaaatttggattattctccacaaaattcttttatttctaaaacgcctcttgtcacatactagttttgtttctctctttaatgcattatctgtacttgaagtgcttagctgggtatgctggcacatgcctgcagtcccagctacttgggaggctgaagcaggaggatcacttgagcccaggagttggagtccagcctgaatgacataaggagaccccttctctaagaaataaaaataaaaacaaatacttaataaagactctgtctttaggatagagagcatagagatataaagcaaagtgtcttgccaaaaatgagtgttatggtaccaatatttgagtagaatgaagaatcttccattgagtagaaagagaatttgtaacatatctgtgtttgatgtttaaggcataacagcttaataatgacactcttcctcagacaggaagcctgaaatgtcctactttgacctaaagtctagtaataaaactggacatacacaggcaacatgtcattaattctcaaactttaacaaatcatatataacctaatataatggttctcaagtctgtacatcacgtcacctgtatgaaaaatatgaggaaacagagacttcttttacactattggtgaggtggataaattgatagagtctttctggagagaatctggcaatgctaatcaaaatttaaaatgcacatacactttgttccagcagttctatctctagtaatttatttttgccctcatatatccataagacatgcaaataattatatgtgaagattttttttttttctttttctgcagagacagggttttaccatgttgcccagggtgatctggaactcctgagctcaggtaatccacccacctcagcctcccaaagtgctgggattacaggtgtgagccatcatgcctgaccaggatttttttttttttcagcattatttcttttgttgttgttgctgttgttttgagagatggagtctcactctgtcacccagactggagtgcagtggtgcgatctcggctccctgtaacctccacctcctgggttcaagtgattctactgcctcagctttccaagcagctgggactataggcgtgcgccaccacacccagctaatttttgtatttttagtagagacggggtttcaccatatgttggccaggctggtcttgaactcctgacctcaggtgatctgcccacctcggcctcccaaagtgctgagattataggcgtgaaccaccatgcctggccatagcattatttctaatagtgaaaaattggaaacatgctaagtgtctatcaatatagcatgagttagatttatgatgtcaccattcaattgaaacactacatatctcccaaaaagaatggtgttccaatatggaaagatatctaagatttattaagagaaaaagcacattgcagaacactgggatcctatttgcttttttttttctttttttgagacagagtcttgctctgtcacactgcaacctccgcctcccgggttcaagcgattctcctgcctcagcctcctgagtagctgccaccatgcccagctaatttttgtgtttttagtagagaaggggtttcaccatgtttgtcaggctggtcttgaactcctgaactcgtgatccacctgcctcagcctcccaaagtgctgcgattactggcatgagccaccgcacctggccatgaaatttttttttttttttaaagagctgttcatattcttattgcctagaagatgtctgaaattacacccaagaaactctttttgagacggagtcttgctctgttgtccaggctggagtgcaatggcgtgatcttggctcactgaaacctctgccttccaggttcaagcgattctcctgcttcagccttctgagtagctgggactacaagcgcccgccaccacatctggctaattttttgtatttttagtagagacagggtttcaacatgttggccaggctggtcccgaactcctaatctcaggtgatccacccaccttggcctctcaaagtgctgggattacaggcatgagccactgcgcccggctgaaactctttttttttcttttaagatggagtctcgctctgtcgcccagacttgagtgcagtggtgtgatctcagctcactgcaagctctgcctcccgggttcacaccattctcctgccctagcctcccaagtagctgggactacaggctcccgccaccacacctggctaattttttgtatttttagtagagacagggtttcaccatgttagccagcatggtctcaatctcctgacttcgtgatcctcctgcctcggcctcccaaagtgctgggataccaggcatgagccaccgtgcccggccagaactcttaatagtagttatttatgcacgctgggattggaagacatttactttttactggatgtctttccgtattgtgtgcttttttttttttttttttatgtagggcatacattacttaagtaattttaaagcctccataagtaagtgtgatttcctgcccatgtgtttggcaaaaggaattgcattggtggtagacttacattatagtcttacctggagtagcacaggaggacccaaggttaataggtgaacttcgaggcaagccttagcattgaggttgccatcagcattgcttggttgatgtgttcattcttctgggatggattacaacctttactggactttatacttttcaccagtaaggctttaaaaaaggagttgaaacattagagaataattatccaggcagtaatattcactggtaaatagtcttccagcctgtggcccaattggttgattcttttacgttaaagaatgcagcctcagctgctctgcctatggagtaggattcttttatttactttcttaataaacttgcttgcccctggctcccccccaccaaaaaaagaaggcagcctcccttttgcgaatggtaatttcctatagtttcctcgtagaattgtggagttacctatgctgaggttataggttagggtattgagatccagagttgccacttctgaggtgtcacaactgctaatggtaaaaccatttctaaagcccagttcttgtgactttgtccagtgattgcctgttcaccgtttcatgctgccttcccatttgagcattcccaggaggaaggggaggttgccagggacctagtaccatagtccgaccttggaatcgttgaatatgagggaaagcgttggcttctcccttctttctcccaaacattggaagtatttttggctgttaaaaagcaccccttgttccatgtggaatcccttgtttaaaagaagtaaaatatgtacctcctgtcctccacagacctgaggaccagtgtgatctcaagaaggttacaggtaaatgtagatgtctctaactgaaaggtggcttttacaggttagagaaaagagagaaccctgatctgaaggctattttatgaagtaattaaaatgttctaaactttaaaaataactgctcaaataattgtgttgtatagttacttatcaactggaggggctgataagtatttttctaaaacatttttaaggaaattttttcctattttctaatttgctaattttgctcaagtagtttgttagatattgttaatatagatgttggttataactgaatgaaagggaacaactactttgacattttgaaaaacaagcttcattttcttctagtctaatggatgggatcccaatgatatgtttcgatataatgaagaaaattatggtgtagtgtctacgtatgatagcagtttatcttcgtatacgtaagtttgaaaagtttgtttttattttagtgcatttgtctttgattttcatcagcttaatttatgatgaataaatgtttgttagtttttaagttaaacaattacatgaaataatttttctcttattaccaactgtgataaatttccattaaaaaaagggaataaatgtagtttgcctataccctgtttttatgctctaaacaaattttggttttgtctttttttttcttttgagagggaatctcgctgtgtctccaggctggagtgcagtggtgcaatctcggctcactgcaacctctgcatcccgggttcaagcgattctcctgcctcagcctcccgagtagctgggactataggcgcgtgctaccatgcccatctaatttctgtatttttagtagagacggggtttcaccatgttggccaggatagtctcgatctcttcacctcgtgatccacctgcctcggcctcccaaagtgctgggattacaggtgtgagccactgtgcctggccggttttgtcttctaagttgttaaaaaatatctaaatttgcaagggcagagattatggtgaacagtttaaccagtttttgaaatatgttcctctggagaaaaggtaacagaaaaaaaagttagaattttgatttataaatacacagatcactataacttttagttttagttttagttttagtttctgtttttaccagtattctaaactctaaactttcttagtagttgattatgacagatacataaactgtggctttaaaggactcattttgcttttcttttcctcatgtttcagagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactaggtatttaaaggaaatcatgatgcagtattttggatacacaactcaaggtctgtgtgagacggtgtattgttattatatttcctcttcctttaatatagcttaggtagagaatgcaagtagaattggtttaagatctgttagagaaaaggttatggtgatcttggaaaatatgcttttgagagtaagctctgtggagccaagtgttggtatatcacggtgagcaatccaagatcttgaagagcttgttaaaatagttatctggtgggggacacgtgtaacaatcacagcagtacaatatgatttgcttggttaaaggcatgttcaaagtactaggaacatacagaatgaggaggagctagcataacctgtagagtcagagaaaacctcattgaggaggtgacattttgtgataagataatagggtctttgacacttagagaagagttgggagaagagtttatcacctgatgaaaagccatgtacaagcatggctatgagaaaatttggccagctcaggagagggctggttgttgcatgtgtctggaacacaggatctgtgtcaggtgcagcagtggcagttgatagtaggaactgaggtcattaaaggacttggcatgtcatgctaaagagcaccctgttggaaggagatggggtgaataaaccctggggcattgaggactggctgagacacagagaacagttagtgcactgaaatagttcaactgtgagaatttggtaaccacctagttaagggatgagcctgaggtttatttgataactaagtgacttaatggatgtactggtaagagagagaggaaacatggagcaagtttgaggggaaaaacagtgactccgtttgtgcagctaattgcatatgtgggcttgtgggtctttcatttattcataaacgtgttgagaaatacctgctacctatctagtaaagtaagagatgcatcctctcttaaaggcagtcagcttagagtctggtgatttgaattgacatgtccactgatagatgttgacactgtgagactggcggttcagtttgaggtttcatcagcattgccgatattggagccatgaaaaaccaaagaacagccagtgagagaagagatctcagagaaaataaaattgagaaagtgaaggacaaaaaatgttgtgaagatagaccaagattgatggaatcagccatagagaggtcaagtgggatgagaatgagcacgcatctgttaaactttgtgcttaggagcagaatctaagggaagggacagtccagaggttagaactcagggtaagatggaagaacaggggcatctgggagtgaggcagtttggtttagtgtagaacctttttgtaacaagcattcccttctgtctagatgacttttagatatgtttcattggcttggtaccttttagaataaaatgatttagaggatctctcattttcagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggcaagtattttgaccagacttgtcaatatcattgataaaatagttttctaaatacttaaaatacttaaaatagtttacataactgatatgaatgtgcactttaatgatttggtgagtagctttcacttcagcattacttaaaattggcttttgtggatattaaattagtaaaacattgtatatgtcattgacatatatattatttagcatgatgaaatattcatgatgtactaagataaagtgctacatttaacccaagacaatcacttggccaaaaacacttcacatataaagaaattggaaactttgggtaggttctcaattttaaaaacactggataataaaattttttagacataatttatatggaaaattctaacctatgtgcaacactgtggttaatatagatcaattttcattatttgtttctatattatgcttacttcaagaaaggatctgaggtaacttataatacaagacatgatcaagagtcatgtgaagaaagtgactagagaaatttgcttaaaaaacaacaaaaacaacccttagtctaagggtggatgttacagtttagcaacttaagtaaaagaaacctgaatctttagtaggaagacattttttactctacctctaaatctaggttgaatatatcttgtaggttgtggatcttttccataaatcagggatactgaacaacagttctatggatggtatggaaatagtaatagcaatagtatgttactaactttgtgggaaaagagtggacattcaattttagctatttaaatttggaaagttagatgaaaatagagaacactaagtttccaatttcatttgttttcattgagtcttttctccagaattcctctccaaatggacactcttgagtattttcagtacttaatattgggggtgaaatttctttgctcactgaggaaagattttagttgtttataaacagaattttaaagttaaaaaacctgaagggggctgagaaatatatgatacttaagtgtgtggaaccctatggagaggagacctggactgtttgataagattaaggtaagtgatatgtaatgttaaatactagctgtatctttacctaggcatatccatcagtataaatttatttggtgatgactgctttgtagttgcagtatttattaagcagtcgcttagataagtgtttaactgtataaattatttagaaggtctccctttttctagtttaatgaggtcaagactttttttttgaaatagcaatgaatattatcatttgatactcacaggagtcacaaactctagaagagtaatgttttatttctacttaaatgggacttgcttaataagattccaaactgagttctgggttcaagtgtaaacctgatgaaaatcatagataattgtaaggaaccagcatttctaattggatataatagctactgcttattttcgttatgcctcagagttaaaactaatacagtaaataatcttactcctgagtaggaattattgtgatttattatgtgaaattatctagtgtatgttatattcctttaaacaaccagttactgagaaacagttatagaagcaggattaataggcaaagtcttaactgtcttcttcaatagtgtgtatagatcctaattaaccctttgggaacgtgtattcatttaaacagacttaatcttaaggaggttaaagtaaaatgtgaatttatgtcagttaagttatgctaaaacttatcacaaatcaaatgactgtcctcaaagggttaaaatgtacaagaaatcatttttgtcattttactttttttctgtttacttttttccctcatttttttctttagtttttatactttccttcatatcatttgttctgtcaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaaggtacaataccacaatttgttcatgtttttgtttgtctttgtttaactcctatgtgagtttataattacaaaatagtttcctcttcattatttaataacctataatttctgtgttttaactttagtttattaaaactatttctattaaccttttgttcattagagagaaatttgataaatgtgtgaagctataaactctcttgaattgttgttaaaaagggggtttatctctgcctgataattatgcttctttacagccccagaagggtctgccccacagccttccccctccttatttgcactgtatacagtagttaaacaaatgaactttcttcagccagtcttgaacttaggttcattttacagctctttggccaaggtcctagtgaaccttcctattggccataagcagggatggtgttttctgggtcttttttgagagcgacagcccatgtagctgactttgcgtgtctgcccttagattaaagtagttgatttttagaatgccagaagaattctaaatttaactgagtaatttttttaaagttagctttgcaatcttacatagtgaaaggctgctttaatctggaagaagtccttgatctgagataaaattgataaaaacgacatatgaatttgaatatttagctatttctttcctcgtcaaaaataagaataaaatcttgtaattcttattcagtatttggcgctaaatccatcattgccacatatcaaatacagggatatgttgtagaaaggtaacattctaatttaaatgccacccatatattaaaaacctgttttctgaatcataatgtccttttgatactagttctgaatatttgtgttaaaattttaatctgatttgttcattaaaattagttaatattgcttatgttgggactaataaagttttccgcacaaaatgtgtttctcctgcttccctggagaaaactgtattggctacttttaaataaattgttaccatctaagcaggcaggtcatatgactttgactgaagcatctaaccttgaagagcaagttccactgattttcaaggtgacttctttgctcaaaagggccttaatagtggtcactaaatgcaaaattctgttgatatttttcttgtagtccatcatttgagtaagcgatgtttatttaatgagaatatattaaataaaacatgatcattaatgactgtgaacatctttattacattaagatttaaggactgctcatgtattaacttcacacagaaatatactttctgtgtcattcagagatgttgaatatttccatttgaaaattatagtgtataacattagcattcttctaaagatcatgttcgtgtttaaattcctgttggaagccaggcatggtggctaacgcctgtaatctcagcactttgggaggctgaggcaggtggatcacttgaggtcaggagtttgagaccagcctggccaacatggtgaaacctcgtctctactaaaaatacccagctacttgggaggctgaggcaggagaatcacttgaacctgggaggcagaggttgcagtgagttgagatcgtaccactgcactccagcctgggcgacagagacagactctgtcttataaaaataaaaataaaataataattctattggcaacatatattaatttgaagttctaaagagtttggcagccgggtgagagagtgaggagatttggctttgacattagggaagttttcgcttggtgttaacaccagtaggcttctctgatgagggccattctgtccactcttttacctgatagattggtctaatgcacagtagactgatttagaaagagtagtcactagtggcatggcagaatcaataatgtagaattttgacaattcatatagtgctgatttctcccccaaatgtcagttattttggtcatctattaatagactaatacaagtcatccctttaatagaattttcagctcacagcctgctaagcctaagaaactgcttacaggttactgcttactgttttaagccgagttttaaaattgatgatcatgatagaagagataaataaactaaaattttagagaaatttaagaagggtatgtacatatgttttagtggtatcggggtgtatagggattaatagtcttctgtttaaattttttttttctaattttagaagtaatgtagaaaattcgggtcagggaaaggtaaaatatatggaaagttaaaaatattttatcatgtagtcataatttctagtaacatatttctttacaaataagacatagttgaaacagattgctacagttcttttaagagttgacatcttattgttgatttcttaccaccaacttcatccctccctttctttaaaaataaagggaaataataaaatttatttataaaactttgtggcattccacaaaataattctgaaagaattagtatggccaaaaaaatatgtatggtgttttttttttttctatttttaaccaaggaaaaactgtagagtgagtgagtgtgtgtgcatgtgtgtgtgaatgggtgtatttagcagaaaagtagtactgatgaatatcatggaatttatgtgatgttcactgtttcttccttagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtgggggtaggtaacacttgggcataatgatggtactcattttgtcattacactagatataaagagggctgagctacaactctgtttgaggaagtgtaagtatgtatatgttaaaaatagtagaatcaccaggaattgggaaacccatatttttattctgggctctaccacttattcatcatatattaaagcaagtcagacactcattctgaagttgagatttcgcagtgagtaaagtgttaataattcttgcctagtctacattatgggattgtgatgagattcctataaggttcataaatacagatatattgtaaaactataaagttttgtaaagtacctctctaatatgaggcaaacacagtatgtaacactatttggagggaccgtatttccttatctttttagcagctttgtttatcagtacattctataaacatttatttttggcttacattgtagtgtgtttctatagcatctgtatatggcactaattcccaactatatttccataataaggaatatcaaatacaaataaagggtccaagttttatttgtgattagcataaggaatatgctgacagcagctataaaagtataaaaattaggctgggtgtggtggctcacgcctgtaatcccagcactttgggaggctgaggtgggcggatcacaaggtcaggagatcgagaccatcctggctaacacggtgaaaccccgtccctactaaaagtacaaaaaaaattagccgggcatggtggcgggtgcctgtagtcccagctacttgggaggctgaggcaggagaatggcatgaactcgggaagcggagcttgcagttagctgagatcacgccattgcactccagcatgggcaacagagcaagactctgtctcaaaaaaaaaaaaaaaaaaaaagtttaaaaactagacgttgacatgattttacaataaggctgactgcttttgctactttgccaatcagtccttagtgctttgttcccataactgtggtaagcaagagcttacaaagaatacttaaaacaaacaaacaaacaaacaaaaaaaacactttttctcttttaatcagtccagagaacctttaaaagaaacaagatcggccagttgctgtggctcatgcctgtaatcccagcactttgggaggctgaggtgggtggatcacttgaggtcaggagttcaagactggcctgaccaacatgatgaaaccccatctctactaaaaatacaaaattagctgagtgtggtggctatttgagaggctgaggcaggagaatcatttgaacccaggaggtgaaggttgcagtgagccaagatcacaccattgcactccagtctgggtgacaagagcgaaactctatctcaaaaaaaagaaaaaagaaacaagatcttcaagcttaaggaaacaaaaacaaaactcagctgtgttaaatctgtttttagttgctatacatttctgctcagcttcatgtgatgcacattcatgtaattgtatcctaaattcctttgtactttttattttcttccttggtcttcaattatcttaagactaccaagaaaacaaaaattttaaaaatcttcttcagccggtcaggcgcagtggctcacggctgtaatcccagcacttggggaggctgaggcgggtggatcacgaggtcaggagttcaacaccagcctggccaacatggtgaaacgtcgtctctactaaaaatacaaaaattagctgggcattgtggcgcgttcttgtaatcccagctgctcaggaggctgaggcaggagaattgcttgaaccaggacccgggaggtgtaggttgcggtgagcggagatcgcgccactgcactccagcctgggctatagagtgagactccatttcaaaaaaaaaaaaaaaaatctgcttcagctattctgttaatcttttgacattacttagatggtctggaaataaattttgagaataacatgattagaagtgagagagtataagcatagttttggagatacactcagaatagcattatagattttctctttttactaattggaaaaatggcagttgttgaataatagttttcttccgtgacccttgtgacttaaaaaaaaaaaaacactgaaatgaaataatcgaaccattttctctaaacctttgaatctgagctctgcagttaggtttataatggtatatgaaacctattagatatatacttggaagtcatatgggatacaaaccctgcttttattatcttccccttttgactaacttgggtctcaagtttccttaattactgcacagtggaccttgatgttgctataaagaatgtgtagggctgggcatggtggctcatgcctgtaatcccagcactttgggaggccaaggtaggcagatcacctgaggtcaggagtttgagaccagcctggccagcatggtgaaaccccgtctctactaaaaatacaaaaaaattagctggttgtggtggcgagtgcctttaatcccagctactccagaggctgaggcaggagaatcacttgatacatttagttaggagagaaaatcatacttatgttagtaattgctgctgttcttcatatacttgtggttttgattgccagcaaattcctaacattttggaaaagaaaacagtaatgggataaagggtaagggctagagaggacagttttatttacctagatcttcagagaagcctgaagcctcttttaggaagtaacatttgaactgagaatgtaataaatacattttccctttcttctagttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgagggtatgtaacaaagggcttctggatccataatctcagctgtgaaattgaatgttagagggtgatattatatgaaaaaattctaggttatttttattcatagacaagtatttttagtgcacatttaaaagtttatgtaaattttgatgttgtttaatactactaatttaatatagtgtctgtgttacaaaggttaacattcctgggtgtcaaatacctacataaataaaattattggtgtttcatatgacatctgcaaaggaaaaaaagcctctgtttaaatgaaagcattattttccaaaaacataggaaatcaaaattattgttcagtgttttcttgttttgcttttctaacttatctgaattttttttaaaaaattgttttctagctaaagattccaggcttcaagatcagaggcagaactctcctgcagggaataaagaaaatattaaacccaatgaaacatcacctagcttctcaaaagctgaaaacaaaggttagagtttaaagagtcattaagcttaactgtaggaataggaagaagtatgtctaatttcatgcccatacagaatatttttgttcaacatttcttcttactattgtgatagataaatgtattgcttgacaaattccaaaatccaaatttaatatttgaaattattttctgatcttatatcttattctaatttctatcatctcatactaaaaagaatgtgatgttaaagtttaaaaataaacctgtgtcttaacagttcttaattttacaggtatatcaccagttgtttctgaacatagaaaacagattgatgatttaaagaaatttaagaatgattttagggtaagtattgtactaactgatgaatttgagttttagaaaataagcattactaaagatttatctatttataaaaatgcgttatgtatacagtcagaaacatcaaaccatatatgtagaaagcagaacatttttaaagtggtctttgcctatcctttaagtgggataactaaaatcatgagatttggtaacaacaatatgtaggtatcaaatgagagtatagccctgacatttgaaaccaccatagcacagcttactatttgatggtcatttgtactttgttcagtgaagctagatattagtagagcaaggccaagtcattaataatctagtgtggcaaatggaagatgtactggactctggtgttctgaggtagttggagatttatactttgtacacaaatatattgtggtcaaaatctttctgtaacattatttctctgtcttagcacaggctttacttaacatctctccttgattgtcatttcattcttttgcatgttatttactataggtatcgaggtagattttgagaccaaccaataaatcttcttgaaacttagcttcttagaaaggaaaatctaaataccagccttttaaaaaaagtagctgaattaaaggatgagtgaaccaaaggcaaaggtagcctttcctcagcctgtgttttagctttctaaatgttaacaatagcttcattcttgacttattggtaacattcaaaatactacttattatttcatactttagcacatgtatctattcagctttaatgctattaacagttgttaacctaagttttcatttgttggcgggcacggtggctcacacctgtaatcctagcactttgggaggccgaggtgggcagatcacctaaggtcaggagttcgagaccagcctggtcaacatggtgaaaccctgtcttgaccaaaaatagaaaaattagctaggcatggtggcgcacacttgtaatcccagctacttggcaggctgaggcaggataatcgcttgaacccaggagacagaggttgcagtgagccgagatcacaccactccactccatcctgggcgacagagcaagactgcatctcaaaaaaaaaaaaaaaaaaaaaaagtttttcaatttgttaaacaatagttaacacatacaaatgatacaaagaatattgaatatgatcatgtgcccactacccagcttagtaaataaagcattctaacacagttaaactcctcttatgtatctgcccctcctcagctgcttccccctgtttccttccaaaaggaagggtttcttttctgtgcagttctttatatttatactgcatatgaatatatctgtgagcaatagatgatattttgcataatcttaaatttgctataaagtcttttttttttttttaattgatcattcttgggtgtttctcgcagagggggatttggcagggtcataggacaatagtggagggaaggtcagcagataaaaagtgaacaaaggtctctggttttcctaggcagaggaccctgcggccttccgcagtgtttgtgtccctgggtacttgagattagggagtggtgatgactcttaacgagcatgctgccttcaagcatctgtttaacaaagcacatcttgcaccgcccttaatccatttaaccctgagtgacacagcacatgtttcagagagcacagggttgggggtaaggtcatagatcaacaggatcccaaggcagaagaatctttcttagtacagaacaaaatgaaaagtctaccatgtctacttctttctccacagacgcagcaaccatccgatttctcaatcttttccccacctttcccccttttctattccacaaagccgccattgtcatcatggcccgttctcaataagctgttgggtacacctcccagacggggtggtggccgggcagaggggctcctcacttcccagaaggggcggccgggcagaggtgccccccacctcccggacggggcggctggctgggcgggggctgaccccccacctccctcccggatggggcggctggccgggcgggggctgacccccacctccctcccggacgggttggctgccgggtggagatgctcctcacttcccagacggggtggctgccaggcggaggggcttctcacttctcagacggggcggctgccgggcagaggggctcctcacttctcagacggggcggccaggcagagacgctcctcacctcccagacggggtcgcggccgggcagaggcgctcctcacatcccagacggggcagcggggcagaggcgctccccacatctcagacgacgggtggccgggcagagacgctcctcacttcctagacgggatggcggccgggaagaggtgctcctcacttcccagactgggcagccgggcagaggggctcctcacatcccagacgatgggtggccaggcagagacgctcctcacttcccagacggggtggcggccgggcagaggctgcaatctcggcactttgggaggccaaggcaggtggctgggaggtggaggttgtagcgagccgagatcacgccactgcactccagcctgggcaccattgagcactgagtgaacgagactccgtctgcaatcccggcacctcgggaggccgaggctggcagatcactcgcggttaggagctggagaccagcccggccaacacagcgaaaccccgtctccaccaaaaaaatacgaaaaccagtcaggcgtggcggcgcgggcctgcaatcacaggcactaggcaggctgaggcaggagaatcaggcagggaggttgcagtgagccgagatggcagcagtacagtctagcttcggctcggcatcagagggagaccgtggaaagagagggagagggagaccgtggggagaaggagaaggagggggagggggagggggggagagggagagggacaatgatgtcttgctgtaggtattcttccccatttgaattttttcctcagcattattttttttaacatcattcagtctcctcttatactacacttggattgaatttaatatctcatgaagaaaaaacatttctactttgaagcatgtgaattagcatgtttttataacagctttattgagatataatttacatatataaataaaccgtttaaagtgtataaatcagtggtttttaatgagatataatttacatatataaatcaaccatttaaagtgtataaatcagtggtttttaaaatattcacaatgttgtacaaccgtcttctcagttgattttaaaacatactcttcacccccaaaagaaaccccgtgcccagtttagcagtcgttccacatttgcctccagcccttctctttcccctactcccaaccctaagcaaccgttaatctactttctgtctctatggatgggcttatttggggcaaattccatttcatacaaatggaataataaaatatgtggcttttatgactggcttctttcactcagagtagtgttataaaagttcatccatgttggagcatgtttcagtacttcatttctttttgtgactgactaatattccttgatgtggataataccacattttgtttatccattaatcagtttgtagctatttgtggtgttctcactgtttgactattctgaataacactgccacaaacatgagtgtgcagtttttttctcgtcctatcttttcatttcttttgtgtacctacctaggagttgaattgctgggtcatatggcaactgtgtttaaccttttgaggaactaccaagctatttgccaagatatctacactattttacattcccaccagcagggtatgagggtttctgtttctccacatccttgctaacacttattgtcttgtcttttttgattatagtcatccttgtgggtgtgaagtgttaacctcattgtggctttaatgtgcagttctttcatggctaatgatgttgaacatcttttgtgtttattggccatttatatatcttctttggattgatgtctgttcaaatctttacccattttaaaaattgagttgtctttttattattgggttgtgggagttctttatatattgtgtgtacaagtccctgttagatacatggtttgcaaatgttttctcctgttctgttggttgtctttttactttttcatcccttgaagcacaaaaatttttaattttgatgaagtccaatttatctgattttgaagtaagcttttggtgtcgtatctaagaaaatactgtttcatcaatcattaaggtttattactcttctgggtttttttaagaattacatttagaggtgtgatccatttggagcaactttttttttcttttgacacagaatctcgctcttttgcttaggctggagggcagtggtgcaatcttggctcacagcagcctcagcctcctgggctcaaatgagtagctggtactacaggtgtgcaccaccacaccttgctattaataacttttgtatttttttgtagagacagaatttcgccatgttgcccaggctggtctcaaacacttggactcaagtgacacgcccacctcagcctcccaaagtgaaaaattgctttcaccttgcactgcggactcgccctgaattctttcttgtgcaagatccaagagccctctctgggggtctggatcgggacccctttcctataacaatattatgagaataacatttgattttttttaagtgaaacaaattgttattaaaaaattaaaaaaggtcataggagagtgacttggtgctcagcccattttgagcagttatttaatatagcataaggtggggttcaaattcattctttatattaattttttatttctaattgacacataaccatacacttataaccatttttactgtgtaagttcagattcattcttccgtatgtaggtattagttgtcccagcaccatctgttaaaaagactattcttggccaggcacagtggctctcaacgcctgtaatcccagcactttgggagtcccaagcaggcagatcacatgaggtcaggagttcgaaaccagtctgaccaaatggtgaaaccgcatgtctactaaaaatacaaaaattacctgggtgtggtggcgcacacctgtagtctagtcccactactgtagtggctgaggcaggagattcgcttgaacccaggaggtagaggttgcagtgagctgagatcatgcactccagtgtgggcgacagagtgagactccatctcaaaaaaaagactattctttcctccattgaattatcttcacatgcttgttggaagtctgttgactacaaatgtgaaagtttattactggactctgaattgtcctccactgaatctctatgtcttatccttatggcagtaccatactgtcttgattagagttactgtattttaaaaggctgtactttttcagttagcagaaaacattttagctatcagcacaactttctgtaaaccttcattaatgcttgacttaaattccaagaaggagcaacataaaaagtcttatctctttaggagttttagtcttactacttttaggtgcctgaataaccaaatgtattatttagcctcttactaataactccttgatccataggggcataccaggaagaaaagaagtggtttttaaaaaatgagagtgggccgggcacggtggctgatacctataatcctaacactttgggaggctgaggcgggtggatcacttgaggtcaggagtttgagaccagcctggataacatggcgaaaccctatctttattaaaaatatataaattagccgggcatggtggcacatgcctgtaatcccagctactcaggaggctgaggcaggagaatcacttgaatccaggaggtggaggttgcagtgatccgagattgcatcagtgggcgacagagcgagaatctgtctcaaagaaaaaaaaagagagtggaaaaaaaaaatatgtgtcccagaacttaaattttaattaaaaaaaaataaaagagtgaactttctaattgttctcttcagataatataatgttattctcttatgttttattgcgtatttcctgtgtaccagatgctgttcttcatgcttgtatgttaaatcttgtctaacatctctgtcaagcaagttctgtttgtatctgcactgtgtatattaggcagcttgggcaaagagaagttaagtaatctgcccaaactcacatggctagtaagtaagagggctgaccatctggtgtttaagcttctagcagtgctttgaatagtaactaatgcatagtgcatgctgcactgtcagtcagtgattcattagagctaacttcatgacatgctcatagccccaaactgcatttgttcacaaatatctgtagtccttcatttaggcagaaatagaaataccttgtgtgtttgttgttccttcccttttgagccatatgcagagtgctgatagctttatttgtgtaagaattgctagtaatttgatctgttttgggttaataatgtgggttttagaggtaaatggacctaggtttgaatgttggcctctatacatcatgtgcgtaacattgtggcatgctatctacttcccccaagccaaaatgggttaattttagaacctgcttcatagtgttcctgtgagagctcgatgagatattgcctataaagtgtttagcatagtgcctagcacatggtatgtattcaatacatgttcattcttactagcaaaatatagatgacccagtattgtacagagtatgtacaatggtgtcattgtaccatttcatgtggagtcacataagaatttcagttttctgctgggcatgatggctcactcctgtaatcccagcactttgggaggctgaggtggatggatcagctgaggtcaggagttccagaccagcctggccgacatgatgaaaccccatctctactaaaaatacaaaaaattagccaggcgtggtggcaggtgcctgtaatcccagctactcgcaagactgaggcaggagaaatgcttgaacccgggaggcggtggttgccatgagttaagatcgtgccgctgcactccagcctgggcaataagagcgaaactccgtctccaaaaaaaagaaaaaaaaagaacttaagttttccattagatttagtatagtgcagagaggaaatacagcagagtgctatattccatatatagcaatatagcattagaacaatatattccaatacagcagagtgctatattcagataccaactagtggacttgctatttgtaagatggcaataatagtatctacatcaaatagggctgttgtgaagactaaatgaataagtctataaatagtttagaacagtgtctggacaggtacagtggctcatgcctgaatcttagcactttgggaggctgagacaggtggatagcttgagctcaggcattaaagaccaacctgggtaacatggtaaaaccctgtttctacaaaaaaatacacacattagccaggtgtggtggcacatgctaatagtaccagctactcaggaggctgaggtgggagaatcacttgagcctgggagatggaggttgcagtgaggtgagcttgcaccactgcgctccagtctgggcaacggagtcagaccctgtttggaaaaaaaaaaaaaagtgtccaacccatagtaagaaatgcagatgtgtttgacattgtaagaaaaagcaacaccaaaagtctgatttttgccttcactcaagaactcttatgataattaaactccgaagtccttggcaatatatatagttggtctgttatgtggatcgcctctactaaagatttttgtgaacaaatgaaagtttaagtagtaagttcctacatcgtgacttaaattgccagtgtgcccacataaataccctgtcaacatttgcccttagccacttgactctttagctatattggtaatgcagtaaagcttgcgatgcgccagagttgcataatgctgtttgccatgacaccaagagccttggtaatgaaaccattgaaattggtttgcctatactgaggctgaagaggtatcttggctctctaattttaaggcaacctttttggctgtgtaggtttctctttagcttgtttctcaccacctggggctgtggcttaggtccgttgtcctaacctgtggcttaggttctgtttttgttgcttgtacttgctcccccttttttcagccattcctgttttctttcttttgtagaggatgccatcttaaatcatcttcagccagtggtagcattttattttttctggtctgcaaacttaaaaacctcatcacttattttgctaatatctttgtcttctgttctttttgatggtccttggttttgcagtctactttaaaggtttttatttttttatgggtacatagtagacgtattattcatagggtctgtgagatatttagataaaggcatataatgtgtaataatcacattagggtaaatggggtatccatcaccatcatcattcatcatttctttgtgtaatgaacgttgcaattgtactccctcagttattctaaaaagtacaacaaattaatgctgactgtagtcaccctgctttgttgtcaaatactagatcttattcattctttatttaactttttaaattttaaacttattttatttatttatttttagacggagtctcactctgtcgccaggctggagtgcggtggcgcagtctcaactcactgcaacctccgcctccagggttcaagtgattctcctgcctcagcctcctgactagctggaactacaggcacgtgccaccacgcccagctaatttttgtatttttagtagagacggggtttcactatgttggctgggatggtcttgatctcttgaccttgtgatccggctgccacagcctcccaaagtgctggggttgcaggcgtgagccaccgtgcccggcctttaaaattattttaaatcattttaatatctttttcatttctgcctccggtcctgcagagttcttattcgttctttctaaattttctttgcacccactaatcacctcatttcccttcttctccccattacccttcccaacttctggtaaccattctgctatctccatgtgttcaattgtttttatttttagtgcctgcaaacgagtaagaatatgcaaagtttatctttctgtccctggcttattttacttaacataatgtcctccagtgccatctacattgctgcaaatgacaggatctcattcttttttatggctgaatggtaatctattgtgtatatataccacattttctttctccatttgtctgtcagtggacacgtaggttgattccaaatcttggctgttgtgtatatagtgccgtagtaaacatgggagtgcagatattccttcaataaactgatttcctttctgagtatatacctagcagtgcaattgctggatcatatggtagctctatttttagttttttgaggaatttccatactgttctccatagtggttttaccaatttacatgtccaccaacagtgtgtgaaggttcccctttatccacatcgttaccagcatttgttattgcctgtcttttggataaaagccattttaactggggtgagatgatatcttgttgtagttttaatttccatttttctggtgatcagtagtattgaatacctttcatatacctgtttgccattcataaataacgatgaggtcttgctgtttggcccaggctggtctcgaactcctgggctcaagcaatcctcccaccttggcttcccaaaatgctgaaattatagttgtgagccactgcacctggccttgtatgtcttccttttttttttgttttgttttgtttttgagacagagtctcactttgttgcccaggctggagcgtagtggtgtgatcttggctcactgcgccctacacctcccggattcaagcaattctcctgcctcctgccaccatgtctgcctaatttttgtatttttagtagagacgggatttctccttgttgcccaggctggtcttgaactcctaacctcaggtgatttacctgcctcagcctcccaaagtgctaggattacaggcgtgagctgctgcgcccagcctgtatgtcgtcttttgagaaatgtctattcagatcttttgcccatttttaattgagttactaaaattttccctatggagttgcttgagtgccttttatattctggttattgatcccttgtcagatgagtagtttgcaaatattttctcccattctgtgggctgtctcttcactttgttgatggtttcctttgctgtgcagaagctttttaacttgatgtgatcccatttgtccatctttgctttggttgcctgtacttttggggtattactcaagaaatctttgcccagagtaatgtccctgggagtttaatgttttcttttagtagtttcatagtttgaggtcttagatttaaatctttagtccattttgatttgatttttttttaatatggtgggacacaggggtctggtttcattcttctgcatatggatatccagttttcccagcaccatttattgaagagactgtcctttccccagtgtatgttcatggcttctttgtggaaaatgagttcacttagacgtatggattcatttctgagttctctgttctgtttcattgatctatatctttttttatgccagtaccatgccattttggttacaataatttgaagtcagataatgattcctcccgttttgttcattttgctcagtatggcttttgctcttttgggccttttgtggttccctacaaattttagaattatttttgtctacttctgtgaggaatgtcattggtattttgatagggattgcactgaatctgtagattgctttgagtattatcaacattttagcaatattaattcttctaatccataaacatggaatctcttttcatgttttttctgtgtcatcaatttcagtgttttaaagttgtcattatagaaatcttttactcatttggttaagtttattcctaagtattttattatatttgtagctattgtaaatgggattgcgtttaaaaaatttttcagattgtttgctgttaaatataaaaatgctcctgatttttgtgtgttgatttttgtatcctgcaattttactgaatttgtttgtcagttctaataggtttttcttttttggagtctaggtttttccaaatgtaagatcatattatctgcaaacaaggataatttgacttcttccattccagtgtggatgctttttatttctttctgttgtctgattgctccaattaggacttccgagtattatgttgaataacaatggtgaaagtgggcatccttgtcttgttccagatcttagaggaaagcctttcagtttttcccttttcagtatggtactagttatgggtctgtcatatatggcttctgttttgttgaggtatattccttctatacccagttctttggggtttttttgtttgtttgtttttgagatggagtctcactctgtcacccaggctggagtgcagtggcgcaatgttggctcactgcaagctccacctcctgggttcatgccgttctcctgcctcagcctcccgagtagctgggactacaggtgtccgctaacacgcccggctaattttttgtatttttagtagagacggggtttcaccgtgttagccaggatggtctcgaactcctgacctcatgatctgcccgtctcagcctcccaaagtgctgggattacaggcgtgagccaccacgcccggccaagggttttaatcataaggggatgtggcattttatgtgatataaattatatatttatatcatgtgatatatatttatatcatacacagtataaataatatatatatatattttttagtctttgtcttttattctgttaagatgtaccatgtttattgatttgcgtatgtcgaaccatccttgcatccctgggatgaatcccacttagtcatgatgaatgatctttttaatgtgttactgaattcggtttgctagtattatattgaggatttttgcataatgttcttcagagacactggcttctagttttccctttttgatgtgtcctttggttttgtatagggtaatagtggccttgtagaatgagtttagaagtattccctcttcctgtattgtgttggaatagtttgagtaggattggtattagttcttctttaaaggtttagtagaattcagcagtgaagccatcaggtccatggcttttctttgctgggagactatttcttatagctttgatctcgttacttgttattggtctcgttacttgttattgtatttgggttttggatttctttgtggttcagtcttggtaggttgtatgtgtctaggaatttatccatttcttcaaggttttccaatgtatcagcatatagatgctcatagtagtctctaatgatcctttgaatttcggtggtaacaattataatgtctcctttttcatctctcattttattatttgggttttctcttttttttctgagtctggctaaaggtttgtcagttttgtttatctcttcaaaacaatttactgttttattgatcttttgtattttcttcatttcaattttatttatttctgctttgattttttttatttcttctactgattttaggttttgtccttgcttttctagttctttaggatgtattggcagatgaagtttttccacttttttgatgtaggcacttactgctgtaaacattcctcttattgttgcttttactgtatcctataggttttgataagctgtgtttccattttcatttgtttcaaggaattttccagttttcttcttaatttcttcatggacccactggtcattcaggagcatattgcttaattttcatgtatttgtatactttccaaagttcctcttgttatctagtgttattttattttatttttatttttgtttttttgagatggagtctcgctctgtcacccatgctggagtgtagtggcgcgatctcggcttactgcaacctctgcctccccagttcaagtgattcttctgcctcagcctcctgagtagctgggattacaggcatgtaccaccactcctggctaatttttttttgtatttttagtagagagggggtttcaccatgttggtcaagctgatctcgaactcctgacctcagatgatccacccaccttggcctcctaaagtgctggaattacaggcatgagccaccgtgcccggcctctagtgttatcttattgtgatcagagaagatagttgatatgattttaacttttttgaatttttatttatttatttgtttgtttgtttgtttgtttgtaacagagtctcactctgttacccaggctggagtacatgtcatgatcttggctcacctgcaacctccgccttcctggctcaagcaatcctcccaccttagccttccaagtagctgggactacaggcacatgccgtcacatatggctgatatttttggattttttttttttttgtagagatggggctttgcgatgtgtcccagggttgtttcgaactcctgagctcaagcaatccacctatttcggcctcccaaggtgctgggattacagacatgagccactgtgccacgtcaaatctttagacttgttttgtggcttaacatagggtctatctttgagagcaatccatatgttgaggagaagaatgtgtattctatagctgttggacacaatgttctgtaaatatgtattgggcctatttggtctatagagcaaattaggtctaatgtttctttgttgattttctgtctgaatgatctgtccattgctgagagtggggtgttgaagtttccgactgttactgaggtctgtttctcttttttgctctaataatgtttgctttatatatctggatgctccagtattggttgcatatgtatttatacttgttataacctcttgccgaattgatccctttatcattatacaataatcttctttgtctgtttttatagactttgtctcaaaatctattttatctaagcatagctactcctgttcttttctggtttccatttgcatggaatattgttttccagctcttcaattttagtctatgtgtgattttataggtaaagtgtgtttcttgtaggcaatggatctttggtttttttttttttttttttgagacagagttttgctattgttgcccaggctggagggcaatggcgctatctcagctcactgcaacctccgcctcctgagttcaagcgattctcctgcctcagcctcccaagtagctgggattacaggcgcctgccaccaagcccagctaaattttttgtattttcagtagagatggggtttcagtatgttcgtcaggctgttcttgaactcctaacctcaggtgatttgcctgccttggcctcccaaagtcctgggattacaggcgtgagccaccgcacccagccttttttttaaatccatttagccactctgtatcttttgattggagagtttagtcgatttacattcagtgttgttactgattagtgaggacttaactactaccattttgttacttattatctggttgttttgtagtcctactccctcccttcccccttcttttttacttcctcttcgctccttttttccctccctcccttccttgttttgaaagtgattttctctggtggtatgttttaatttcctgctttatattttttgtgtatctgttgtaggtgtttttgatttaagatcaccatgacagctgggtgcagtggttcacacctgtaatcccagcactttgggaggccgaggtgggtggatcaagaggtcaggagattgagaccagcctggctaacatggtgaaaccccatctctactaaaaatacaaaacttagccaggcgtggaggcacgtgcctgtaatctcagatactcaggaggctgaggcaggagaattgcttgaacccaggaggcagaggttgcagtgagtcaatattgtgccactgcaccccagcctgggcgacagagtgagactccgtctcaaaaaaaaaaaaaaaaaagagatcacataagggttgcaaataacattttataacccattattttaaaccaatgacaacttgaaactttgattgcaaaaacaagcaagcaaagagaaaactaataaaaactctacacttcatctgcccgctttttaacttttgttgtttttatttatatctttattatactatgtcttaaaaaactgtagttataagccaggcgcagtggttcacgtgtgtaatcccagcactttgggaggctgaggtgggcggatcacctaaggtcaggagttcgagaccagcctagccaatatggcaaaaccccctctctactaaaaatagaaaaattagccggacatggtggcgggtgcctgtaatcccagctactcggaggctgaggcaggagaatcacttgaacccaggaggcccaggttgcagtgagccgagagtgcgccactgcactccagtctgggcaacagagtaagactgtctcaaaaaacaatacaaaacaaaacaaaaccctggcctagtggctcacgcctaatcccagcactttggaaggcaaaggtggggcgaatcacaaggttaggagttcgagaccagcctgaccaacgtggtgaaactctgtctctactaaaaatacaaaaattagccaggcgtggtggcacgcacctgtaatcctagctactcaggaggctgaggcaggagaatcgcttgaacctgggaggcggaggttgcagttagccgagatcgcgccactgccgtccagcctgggcagcagagcaagactctgtctcacaaaaaaaaaaaaaattgtagttcttatttttgaaaggttcattttttattcttcctgctcaaaatatgagtagtagtttatacaccacaattacagtgttacaatattctgtatttttctgtgtacttgttaccagtgagtttttgcaccttcaggtgatttattattgtttgttaacatccttttcttgcagattgaagaactttttttttttttttttttttttgagacagagtcatgctctgttaccagcctggagtgcagtggtgccatcttggctcactacaacctccaactcccaggttcaagcgattcttctgcctcagcctcccaagtagctgggattacaagcatgtgccaccacgcccagctactttttgtatttttagtaaagacggggttttgccatatttgccaggctggtcttgagctcctgacctcagggtgatccgcccgccttggcatcctaaagtgctaggattataagcgtgagtcatcgtgcccaacttggttgtttattttcaaatagcctgaattcaagctcactaatgttttctgctgcttgatacatttctgctattgagagactgatgcatttttcagtttgtcaattgaatttttccactttgggatttctgcttgattctttttactaataattattgcagtctcttttttaaatttataggattctgaatttgttctctgtattatcttggatttcgttgaactttctcaaagcattcagcttgaattctgtctgaaagttcacatatctcttatcacttgggaattggtcactggtgtcctttatttttagttcatttggtgaggtcatgttttctcagatggccttgatgcttgtggatgttcatcagtgtctgggcattgaagagttgggtattctgttctttgtagtctggttttgtttgtacgcattcttttttttttttttctgtttttgagacagagtctcgctctgtcgcccaggctggagtgcagtggcacagtctttgctcaccgcaacctccgtctcccggattcaagcaattctcctgcctcagcctcctgagtagctgggattacaggtgcgtgccaccacgcctggctaatttttgtatttttagtaaatatggtgtttcaccatgttggtcaggctggtctcgaactcctaacctcgtgatctgtccgccttggcctctcagagtgttgggattacaggcgttagccactgcatccggctcccattcttcttgagaaggtttttcaagtattcaaagggaattaagtgttgtcatctaagtcttcgctcactgcagccatacatgcattagagggcaccccaagactagtaatgttgtgactctgtagaggtatcaccttggtagtcttggggaagatctgggagaattccctgtattaccaggcagtctcttgtcctcttacatttctccaaacaaatggagtctctctttgtgctgagctgcttggagtttggggaagggtgacacaagcactgccatggccaccgtcactggaactgtacttggtctcacccaaggcctgtggcagctattttctggccaccactgatgttaatttaaggcccaagggtgctttagtcagtaggtgaagaatcctgcaagaactgggtctttactttcagtgcagcaggttcccttctggcccagggtgtgtctagaaatgctgcccaggagccagggcctgggatcgggagctttaggaatctgctttattgtactggggctgagctggcacccacttgcaagataaagtcctttttactcttctctcacctcaagcaggtgggtctccccatggacaccacagctgtgaatgtgcggggtcatatctgaagctggcacaatacgacatggcaccttgttttttattcaaggcacaagggctctttagtcagctggtggtgaatcctactaggactaggtatttcccttcaaggcaatgggttcccttctggtccagaatatgtctagaaatgtcatctgggagctatggcctagaattgaggcttcagaactatgcttggtgctttattttactgtggctgaactagtatccacattgcaagacaaagtcctccctactcttccctctcctcccagagctgtgagctgtggtacctggagttgggggaaggctggcacaagcactcccttggccaccctagctggtgtctcagtgggtcacatgtaccccaagtccactgactatgagcccagcacagtaccatgacttgtccaggaattgcagtccttctggtctagactgcctttcaagtttatttaggaccccagaggactttacccacggtggtggggcttaccaaaattaagattcttttggttttttttggcagagtttcgctcttattgcccaggctggagtatagtgacgcaatctcagctcaccacaacctccgcctcccgggttcaaataattctcctacctcagcctcctgagtagctgggattaccggcatgcgctaccacctctggctaatttttttgttttttagtagagatgaggtttctccatgttggtcaggctggtcttgaactcccgacctcaggttatccgtccgcctcggcctcccaaagtgctgggattacagaccatagtgcccagcccgaaattcagattctaatcactgggatggacaattcccctctgactagggctagtctaaatactccctctgtgggtgctggctgaattctgtcctatgctgctttccactgtgacagggcagcactgagtttcaatgcaaaatcccacagtcatttctctctctctcccccgagcacacagattctttctccaccccacactgcattgtgggggaatgtcaggggtgttggaggggcagttcaagactatcttccttatcttttttggtgtctttttccttgataggatgtcaaaactgggtactgtgatcgcttacctaatttttggttcttatgaaggtgctttcttgtgtggatagttgttcaatttggtgctccttgttggggatgatcactggaaggttctgtttggccaccatgctctgtctcttctcccctgccatctcctttttttacttaggggtttagaatgtctaactgaccaatatgtacagtcaggtctcattctgaattcacctacttaatgaccttccaagctgactaggcccagcgcttagtccagcctccatgacggtccctccacatcctaattagcctccctccagttcatttcacacaaagctgctgtgttcacctttctgaactataaatctgcccagtactctaccctacttaaaattccgtatagactgcccatttgccctgagaattaaaagccaaagtcctaaacgtagctttttaaaacttttttttttttttttttaatttttagatggagtcttgctctgtcacccaggctggagtgcagtggtgtgatcttggctcactgcaacctccgcctcctgggttcaagcaattctcatatgtcagcctcccaagtagctgggatttacacgtgtgccatcacgcctggctaattttttttttttatctttagtagagacggagtttcaccatgttggccagtctggtcttaaactcctgacctcaagtgatccacctgccttggcttcccaaagtgctaggatgataggtgttagccactgcacgcagccctgaacatagcttttaagttcctttattgtcatattccttttgacgagtctatcattttctgactcacttgtacatgtgtgtctcacccttggtccagccattggtgcttttctttacttctttatttttgttattttattttattttattattattttttaaatgagacagggtatcactatgttgcccaggctggtcttgaactcctgagcttaagcagtctgcttgtctcagcctcccaaagggctggaattacagtgatgagctactgtgcccagctcattggtgctatctttttttttttttttgagacggagtctcgctctgtcacccaggctggagtgcagtggcgtgatcttggctcactgcagctccacttcccaggttcacaccattctcctacctcagcctcccgagtagcagggactataggcgcctgccaccatgcctggctaatttttgtatttttagtagagatggggtttcagcgtgtgagccaagatggtctcgatctcctgacctcgtgatccgcctgccttggcctcccaaagtgctgggattacaggcgtgagccaccgtgcccggcccccattggtgctattgttttatgtgatagagccagcttctcccttttctttggatttttaaacatactcttccttttacttagactattctccatcccaacacctttcctaaacttctttcacaccttagactagctgacactttactgagaaacctttcttttttataggttgctttttctatagactctcttagcatttactcattttattgtgaagtgtctgatcttatttaaatgacaagtataagaggatagaaactatttcatatttttctcacccagcaggcacaatttctgacatgtggtaagcactcagtaaatattgaactttagaggctaggacatttgagtgctttggtgactgtggttgtgctatataggtactctgttattgttagtttatagtaaaagcattactcttaaagtatgaaaaaagccttattcagaacatttcatgcgtatagttaatattacgtagcttgtgctcatggcaaaaatgtattactaaagttatttaagatatttaagtataattgtttcctttatttagttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagtgagtaaacctggaacttagaccatcctgttactcaattaacttttttttttttaaaggcatttaggtccttccaactgtgaagaatccatctggacttttagactactttatacattgcccttagtttacaaacagctagtccaaacaaatgacatcttaagtaaatgaggttattgcaccctgtgctactcttctgttcttccccttttttgtaccccagggctagaaaaacaaggcataaattaagaaaagtttttctgtaaatgaacaggagttgaaaaattatcaattcaggggacctatctttactggattccactcattagtcaccctcactgtgctgctaggttgaaaaactgccactgtcaaggagagaagcatgcggtgcttctacttggaattcaaaatatttttcatcagaaactgtgttttagttaatgtttagatttgttaagatagacttaattctgcacattcagtatattaattaaatggacttttaggggctaacctcagaacttaactaccattgacttaggtgtttgggtaccaaacaatccagttaaagctgaagttttggaatgcagcttattgataaattggggactgcttattcttgatttgaggcaatttttttttacagccatgactttttccaggtatgtcatgtaaaatatcttctcacataagaattactgcatgctagaatattggtatgttgactggtagctcatacctataatcccagcactctgggaggtccaagcaggtagattacttgaggttaggagttgaagaccagcctggccaacatgtgaaaccctgtctgtactaaaaatacaaaaattagccaggcatggtggtaggtgcctgtatcccagctactcgggaggctgaggcaggagaattgcttgaacccagaaggtggaggctgcagtgagccgagatcatgccactgcactccagcctgggtgacagagcgagactctgtctcaaaaataaataaataaataaataaaaggatactgttatgttaagaattgcttttaaggatatttcataagtagctactgtcttttcagctcaagtgtttgttgattggccaggcgtggtagctcatacctgtaatcccagcactttgggaggctgagtcaggcagatcacttaaggtcagcgtggccaaaatggtgaaaccccatctttactaaaaataaatattaaaaaaaattagctgggcgtggtggcagtctcctgtaatcccagctaatcaggaggctaaggcaagagaatggcttaaactcgggaggcagaggttgcagtgagccaagattgcactgctgcactccaacctgagcaacagagtgggactctgtgaaggaaaaaaaaaaagtattttttgattgcctttgagaggaacggttgtatattactcagatttttaaaaaattgttcttttatggctgtattctttaagggattaaggaatgggcaatataagtgtatatgtttcaataaaaacgattagtgatcttctagtgagaacagtttaaatctatatttagcaattttttttaaattgtcaggtatggaagattttagagcaacgtaaagtccatgtagatttcactggcctttatattttttttaggcaagttaggaaatcaacattgaatcccaatgcaaaggagttcaacccacgttccttctctcaggtaggtttattactttctttgaggttatctagtcccaaaaaaagaaaaattattagtaatagtccttcttccatacctgccatctgaattttgttttagtgtgctgaaccaaccttctttcttttttttacatggccattaatgaatactttttaaacattaaaaaaaggtctttgttttgtcatcaattagatgtgatcttgggcaaatctttgaatttctctgacccagaatttgacgatggttggctagctaggctgtcaggtttatagatacgtcctctgcacctgagggttttgcatcactggattcaaccaaccatggatcaaaaacatagttaggataatctatactgaacacatgcagacgtttccttgtcattattccaaaacaatacagtaaagcatttaccttgttttaggtattataaataatctagagatgatgtaaagtatataggaggatatgcataggttgtatgcgaatactacatgattttatgtaagggacttgagcattccaagactttggtatcttcacagggtactgtaaccaatcccccacagatactaagagatgactgtactattgttattattcgactgagatcataagaagatatatttatttttaatttttaaaaacacttccatcagtttcttaaaaatagctgccactgtttttaatattttttaattgacaaagttttaagttcctactgaaacattttttcttttattgaaatgtgaaaatttatgtgctgtgtttttgttttcaataaaagggacatagttaaagcaagtaaaattagaaagactgggaaaatccgtctttaaattgcaataatagttcatctgttaccttgagataattgaatttattgttgtttttgtagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaagtaagtcatagaatttgatgttcacttagcctccccaattgtttgtatctgacaccaagcactctttaggttttcagtgacttgagggtgtgatggttatgcatatgcatttgaaacagacaggcatgcagagattcagtgtgttgttaagtatgaggacctaaatctgagaatgttttctgtgaaaaagatggtttagatttactgtagtttggggtttgttccttttagctgtgggtatgatctaattttttaatgactaatggagaatcaggaaaccttctcatgcctagctctctagcaatataaaactaagagtgacagaataccttgttattatcataggtgcctaatgttaattttttttttaattctctcaagcctttatacccaatacctatgacgcccatgccagtgaatcaagccaagacatatagagcaggtaaaggtgagaataatcctgcctgtgtttgcttgtagtttgcatgctgcatgaattgagtaactaagtttataatgaataaatagttgtagtttagctctgactttttgatgaggctatgcattggcttttgatgaacaacattacatagatattcacatggattttatgaagaaaaacaggggagaaaaaatgcccatcagttgtgattatatagtatcctcttcaaaaagagtaattggaggcctggtgtgatggctcacacctgtaattttagcactttgggaggccaaggcaggaggattgcttgagctcaggagcccaagatcagcctggacaacagagactttgtctctactaaaattcaaaaaaattagctgggcatggtggcatatgcctgtagccccagctgtttgggggactgaggcgagaggatcacttgagcccaggaagtagaggctgcagtgagctgtgattatgccactgccctccagcctgggcgacagagtgagaccccgtctcaaacataaatactggctgggcatggtggcttatgcctgtaatcccagcactttgggaggccgaggtgggtgtatcacctgaggtcagtagtttgagaccagcctggccaacatggcgaaaccccatctctactaaaatacaaaaattagccggacatggtggcacctgccgcctgtaatcccagctactaggtggggctgaggcaggagaattgcttgaacccgggaggcaggggttgcagtgagccaagatcgtgccactgcacttcagcctgggcaacagagtgagactccatctcaaaacaaacaaacaaacaaaaaacaaacaaacaaaaaaaccagactaattggctggacacagtggctccatgcctgatatcccagctggaggatgacttgaacccatgagttcgagagcagcatgggcaatatagtgagaccctatctcaaaaaaaaaaaaaaaagttaattccaaagctttttgatctgaaatctgatttaaatctgaacttaaatttgaagaagagggtttgctagattaatttactagattgctaaccttgctttatatatacctacagttatttccccaaagccagaatttcttttgaagcagaggggcaactaacttcaaccaatgttaagatcctattagaaggatgtttcggctaggcttggtggctcacgtgtaattccagcactttgagaggctgaggtgggcagatcacatgaccgggagttttaagaccagcctgggcaacatggcaaaaacctatctctgcaaaaaaaaaatagaaatcttagccagccgtcatggtgtgctcctgtagtcctagctacttgggagactgaggtgggaggatcaattgaaaccagaaggtccaggctgcagggaactgtgactgcaccactgggctccagcttgggtgaaagagcgaaaccctgcctcaaaaagaaaaataagatggatgtttctgcattaaaattagggagttgtcgtataatgtagttgcataaactagtattctgtgcttgtgtggttaaagagccttcgtagaaaaaatcccacatttttcttaaaaggaaatcttttggccaggtgtggtggctcacatctgtaagcccaacactctgggaagccgaggtgggcagatcacttgaggtcaggagtacaaaaccatcctggccaacatggtgaaaacccgtctctactaaaaatacaaagatcagctgggcatggtggtgcgtgcctgggtgacagagcgagactccgtccaaaaaaaaaaaaaaaaaaaaagagttcttttaatgttggaaaatgctaaagggttttttttttgccaaccagttaatttagagtgattaactgctatcagttgagaaactatagaaagtagaataatttatacagaaaagacatttctcagtgcccaataattgcctttctgacataaagttttcatttttcctgaattaataagatttcctcaatgtgtttttttgggtgttttgtgtgtgtgtgtgtgtgtgtgtgtgtgtgtgtgtgtgtgtgtgtatgtgtttgatacagggtcttgctttgctgctgaggctggaacgcagtggcgctatcatggctcaatgcagccttgacctcctgggctcaagcgatcctcccttctcagtcccctggatagcgggggctacaggtgcacaccaccacacctagctaatttttgtattttttgtagagatgggttttgccatgttgcctaggctggtctcaaactcctgggctcaagcgatctgcctggctctgcttcccaaagtgcctgcgcccagccaattttctccatgtttgacctaattgtgatttcatagatgttaactaaaactcttaattttcgttttctcagtatgctattttttttttttttagccttggaacatatgaacctgttgaaagaactctgcctgaaataatgtaatcaaattatagagtttaatcttattttgagggcctttagaaattctgagaagaaagtgggtttttttttttactgccattttaatgtagtgttaaggtgttcatgtatcaccagcaggtgtagctgttttcaatgattacttaaaacaatgcaatgggaactttttgttgtcattaaaatataaaaggttactgtagtaagagcaagcatgacagtttggctatctgatgggagagtcacattctaacttcaggaggtactgtctttttaatagaaatgatatactcagagtctgggcacggtggctcacgcctgtaatccagcactttgggaggccgaggtgggcagatcacgaggtcaggagatcaagaccatcctggctaatacagtgaaaccgtgtctctactaaaaatacaaacaattagctgagcgtggtggcaggtgcctatagtcccagctactcgggaggctgaggcaggagaatggcatgaacctgggaggcagagctggcagtgagctgagatggtgccactgcactccagcctgggtgacagagcgagactccgtctcaaaaaaaaaaaaaaaaaaaaaatagtagagaaagggctttgccatgttggccgggctggtcttgaactcctggcctcaagtgatccacctccctcggcctcccaaagtgctgggattacaggtgtgagccactgctcctggcctgaatataccacttttacctatcatcagttgatgaacatttggattatttcctttttctggcaatgagtaatgcttttgtggattttcatgtacaaattttcatatgaggctgggagcagtggctcatgcctataatcccagcagtctgggaggctgaggtgggcagatgacttgaggtcaggagtttgagaccagcctggccaacatggtgaaatcccatctctactaaaaatacaaaaattacactggcatggtagcgtgcacctataatcccagctattcaggaggctgaggcaggagcatcagaatcgcttgaacctgggaggcggaggctgcagtgagctgagatcacaccactgcaccccagcctgagtgaaagagtgagtctcaaaaaataaaaaataaaattttttttcatgtggccttagattttcatttctcctaaagtagaaatgctgtgatggaactgccaaacttttccaaagcagctgcatcattttgtatttctaccagtaatgtacaagtgttccagtttctccacatcctcataaataaccgatatgtctttggtttgggttatgtccattctagtggttatgaagtgtcattgtggttttttgtttttttgtattgttttgagatcgtgcccaggctggagcacagtggcacaatctcggctcactgcagccttcgcttcctgggttcaagcaattctcctgcctcaccctcccagatagctggggctgcaggcatacgccaccacaccaggctaatttttatattttttgtagagatggagcttctccgtgcttcccaggctggtctcgaattcctgagctcaagcgatccccctgcgtcagcctccagagtagctggggttataggcgtgcaccaccgcgctcggcccatttttgtatttttagtagagatggaatttcaccatgttggccaggctggtcttgaactcctgacctcaaatgatccgcctgcctcaccttcccaaagtgctgagattttagacgcgaaccaccatgccctgactataggttatctttttacttgcttgatggtgttctttgtaacacagtttttaattttgatgaagttcaatttatctgtttgttttttcttttgttgctgttgctcctgatgtcatatcagacaaagcattgcctaactcaaggccacagagatttactcctatgaaacgcctataaaactcctatgatttttatagtttagctcttaacatttaagtctacaatctcttttgagttaatttttgtgtatgagatgagagtagtggtccaggtttttccttttgcttgtggatatccgttgtccccacctcatttgttgaaaagactattctttcctcttaaattgtttgtttgtttatttatttttgagatggagtgtcgctctgatggagtggcgctaacttagcttcactgcaacctccgcctctcagattcaagcgattcccctgcctcagcctcctgagtagctggaattacaggggtgcgccaccacacccagctaatttttgtatttttagtagagacggggttttaccgtgttggtcaggctggtctcgaactcctgatctcgtgatctgcctgtctcctggcaccctgggaggctgagaggctgaggtgggaggatcacttgagctcaggagtttgagaccagcctgtaccattatgcctggctaattttagaatttatcttaaagtataaaatgtgaatccaatttatcttgttctaaatgactatccaaaatgttttaaccagttttattagtctgtaatttacatacaagaaaatgctcatctttttatgtttacattttaatgagttttgacaaatatatttgctcatgtaactacttgcttcatcagtgaagatggaaaacattgtgcctgttcctcttctctgtccaactgtactttattaccactagctccagttaaccagtaatctgccttcttttactatagattagatttatcctctttagatttctttttctttttttttttttgattaggtttttttttttctttttttacgtaaaaaaatctttttttggagacgtctcattatattgcccaggttggtctcgaactcttgagctcacctcagcctcccagagtgctaggattacagatgtgagccacctcagccagcccctagattttttttttttttttaataaatggaatcaaacagcgtgtaacagaggtgttcaatcttttggcttccctgggtcatattggaagaagaattgtgttgggccacacataaaatacagtaacactaatgatagctgatgaacaaaacaaaaaaaaatagcaaaacttataatgttttaagaaagtttatgaatttgtgttgggccacattcaaagccgtcccaggacgcaagttggacaagcttggtatataatttcatatgtgtgtcctaaacagtgtagtaatttgaatttcatgttagtatcagcttattcctttttgtttgtttgtttgtttttgagatggagtcttgttctgtgtcccagaattggtctgcaattccactgcctcagcctcccaagtagctgggattacaggcacgtgccaccacacctggctaatttttgtctctctctcttttttttttttttttttttttttttagcagagacgggatttcaccatgttggccaggctggtctcaaactcctgaccccaaatgatccacctgccttggcctcccaaagtgctgggattacaggtgtgagtcaccgtgcccagccagcttattcctttttattgctgggtagcatttcattttatgattataccacagttaatttacccattactagtcgatgggcatttgagttattgccagcttttggctattatgaatgaagctgctgtgagcatttgtgtacaagtgtttgtgtttttatttcttttagttaaatacctagaattggaattgctgaggtatggtaagtgcatatttcatttttttaaaaaatttattttattttttatttatttattttttttgagatgaagtctcactctgttgcccaggctggagttcagtggcgtgatttcagctcatggcaacctccctgtcccgggttcaagcaattctcccgcctcagcctcccaagtagctgggattacaggcgcgcaccaccatgcctggctaatttttttgtatttttagtagagacggggtttcaccacgttggccaggctggtctcgaactcctgaccacaagtgatccacccgccccagcctcccaaagtgttgggattacagatgtgagccaccacacactgcctggtaaatacatatttcaattaataagaaactagcaatcttctaaagtgattgtgtcattttacattccaactgatcaggtacatgtgtaggttccatgtgttctgcatccttgccaacacttggtattgtgttatctttttaatttcaacaggtctaatgggtgtcttatggtatctcattgtgatcttaaatgtacatttctctgatgatgactgatccaggagcacctcatcatgtgtgtgtttgttttcagctgtcaacctttttttagtaaatggttcaaatcttttttccattttatttatttatttatttatttgatggaatctcactctattgcccaggctggaacgcagtggtgccatcttggctcactgcaacctccgcctcccaggttcaagcaattcttacgccttagcctcccaagtagctgggattacaggcatgcgccaccatgcctggctaattttgtatttttagtgtaggtggggtttcaccatgttggtcatgctggtctctaactcctgacctcaggtgatctacctgcctcggcctcccaaagtgctgagattacaggtgtgagccactgcgcctgtcctaataatttctttttgtctcaatgtttctgcctgggtgcactggctcacgcctgtaattccagcactttgggaggccaacctggatggatcatttgagccaacagtttgagaccagcctgaggaacatgacaaaaccctgtctttgcaaaaaaaaaaaagaaaaaagaaaaattagccaggcacagaagcgcattcctatggtcccagctacttggggggctgaggtgggacaatcgcttgagcgaggttgcgggggtttggagggcgatggaggggtgatcgaggttgcagtgagctgagattgcactactgcactccagcctgggcaatagagccagaccctgtctcacaaaaaaaagaaaaaaaagtcatgtttcttttcttactgtgaaaataaagttactacttttagtaaattattttaagttatttatatattctggttacaagtcctttctcagaatattgtgaatattttctcccagtctgcggttttttttgaagagccagtattgttaattttaatgaagccttatttatcaagcttttctcttaaggttcatgcttttttgtatcataataagaaatcttttacgtaccctaggttatgaatgtttttatggttaggtatatggttgatttcaggttaggttttgtgtagggtgtgatgtaaaggtctagcttcattttctccaccataaatatttactcggtttctctggcaccagcctctgttttccattggtggctttattttttttctgttcttgaaacaagagtctcgatcttgttacccaggctggagtgcagtagtgtgaccttggctcactgcaacctccacttcccagggtcaagcgattctgcctcagcctctcgagtagctaggattacaggtgcccgccactacacccagctaatttgtattttttttttttttttttttagtagagacagggtctcaccatgttggccaggctagtctcgaactcctgacctcaggtgatctgctcatctcagcctcccaaagttctgggattacaggcatgagccactgcgcccagccatagtagctttattgaattcagttgactgtattgtatgtgtgtctatttgtgaactgttttgttgtattgatctttgtatatatccttatgccaattctctctttattgctgttactttgtaaccaacctttaagttcatatgagtctcccagttttattctcgtcaaaattactcttattctgcgttctttgaatttgcaaataaattttagaatcagcttgggattgtgcactgaatctttatatcagttctgggagaaatatcttaacaatatggaatcttcattgaggtcatcatatactgctccatttatttaagtcttaagtttcaccagtgttttctagttttctttgtatcagttttgtgcctgctttcttaaatttatcccttaatatttcatctgttttgtgctgttgtgagttatattttaaaaactttcaacgtttgtttattcgtaaatagagatgcacttgatttttgaatattgaccttgtgtcttgatgtgttggtaaacccactgtttctggcagccctttaagacttaaacatacaatcatgatctaatcaccatgttggtgtttttgggttttttttttttgtcttattgtactggtgcattactgaaaaaggcatgagattttgccatgctcccatttttaggggtgagacattgtctttcactattaagcatacagttaggtgttacttcagttcctaatttgcagaggtgggtttgttttctttttaatcatgaatggttgttggattatgttcaaatacttatcatctactaagtatatcatattgaccaggaacagtggctcatacctgtaacctcagagctttgggaggccaaggcaggaggatcgcttgaggccaggagttcaagaccaacctgggtgatgtaggaaaaccccatatctacaaaacaatttaaaaatttgctgggtgtggtggcacacacctgtagtcctaactacttgagaggctgaggaaggagaattgcttgagcccagtagtttaaagcagcagtgagctgtgattgtaccactgtactccagcctgggtgacagaaggagaccctgtatttaaagtgtgtgtgtatgcgtgcgcatagatggatagataataatgtaattccattatggtcatacaaactgatatgaaatgccattttatcatataacaagtgtctttttgtggttgaatttgtttctggatttttcactctgcttcactaatctaataggactaccttctcatccactcactgccaacattgattttttttttcagattaccttgaattttctgtttatttttccatatgaactctataattaacttactactaaaaaaatcagttgcctttttaaaaccaactgatctttaaaatatatcttggctgggcccggtggcaggcacctgtaattctagctacttgggagactgaggcagaagaattgcttgaacccaggaggcggaagttgtagttgagttgagattgcgcacctgtactccagcctgggtgacagagcaagattccctcttaaaaaaaaaaaaaaaaaaaagaaacagaaaagataaatctttttacaataatttgttccaattagggtccaagtcaggcttgcaatttggatttgtttatatgttgaagtctttttttttttttaattgtttcatattgtggtaactttttttttttttttttgagatggaatcttggctctgtcacctaggctggagtacagtggcacaatctcaactcactgcaacctccccctctggggttcaagcaattctcctgcctcagcctcccaagtagcccagccttttttttttgagacagagtctcgctctgttgcccaggctggagtgcagtgatgcgatctcggctcactgcaagctccgcctcttgggttcatgccattctcctgcctcagcctcctgagtagctgggactacattcgcccgccaccacacccggctaatttttttgtatttttagtagagacagggtttcaccgtgttagccaggatggtatcgatctcctgacctcgtgatccgcccgcctcggcctcccaaagtgctgggattacaggtgtgagccactgcgcccggccttgtatttttaatagagatggggtttcaccatgttggccagcccggtcttgaactcctgacctcaaatgatccacccgcctcggcctcccaaagtgctgggattacaggtgtgagccatcgctctcagccttgcggtaactttttattacgaatgtattgagacattaataacctaggccagtcatgtttcatccctacccattgtctcttaaaagctttgagtccactggattattctgaagcaaattctagacattgcatcagtttatccaccaacattttagtgtgtatctttaagttggttttggttttgttttttgtttttgagatggggtctggctttgttgcccaggcttggagtgcagtagtgcaatcatagctcactgctgctgcgaattcctggtctcaaaggatcctccctcctcagcctctcaagtaactgtgactacaggcacatgccaccttgccagcttttcttttcttgtcttgtctttcttcttctttgtttttttgtttgttttttgtttttttttgagacagagtctcaccatctttctatcttgcccaggctagtcctaaattccagggcttaagttatctttctacctcagcctcctaaagtgctaggattacaggccagcactttaggaggtgctggatgagccatcacacccagccaagtcataggtttttttgtttgtttgttttttgagacagtgtctaactctgtcacccaagctggagtgcagtggcatgatttcagctcagtgcagtctctaccaattgggcttaggtggtcctcccacctcaacctcccaagtagctgggactaaaggtgcgcgccaccatacctggctaatttttgtattttttgtagagacagggtttcgaattcctgagctcaagcagtctgcctgccttgactcccaaggtgccaggattacaggcatgagccactgcactcagccctcacagttttaattacagtttttcccttagtttttgtcttgttcatatccagcttgtcttgtatttttttcccacgatctgaattttgctgactgtatccctgtgttgatatttaaagtagacttctgtcccctgtaatctttgtaaactgatagtaaataatgaaggcttgatcagattgggttttttttttttttccccaatgtttcacagatgtgtgtactttcagtgaggagtcatgtaatcagtctttttcctgataggagtagtcagtgagttcctagatgttttatctatccaggagataatatgtccctttagcgccttaatttttttggtgtgttttttagcagccattgatgataattgtctagcccaagatcagttatttccttaggggttgtaaaatggtgacattcttttcctttcatcccttcttcaattattgcctggaatatttctataaagaaaaactttcccatatccagctgtttggttaccctgaggtatagctttcttaggaaaagtaatttaaaatgttaatcatttccctttttaaggcagtcttcaaaataatgagttggttttctgttatcctccaaaggtaaccagtgaggtggtttttttgtcgttggttcttactatcagtataaacttctggaattttttttttttttttaattttttggagacaaggtctggctctgttacctaggctggagtgcagtgggatgatctgggcatactgcagcctcaacttcccgagctaaggcaatccccccacctcagcctcccaagtagctgggactacaggcaagcaccaccgtgcctggcttaatttttgtatattttgcagagacagggtttcaccatgttgcccaggctggtgtcgaactcctgagctcaagcagtctgcctgtgtcagcctcacaaagtggtgggactacaggcatgagccaccatggcaggccagaatcacaataaacttataaattaacttgagaagaaatgattgatgtcttcatgatgttgagtcttcctgttcaagaacaaagtataccttcaatagcatattaaagtttatccttggctggatgcagtggctgacgcctgtaatcccacctctttgggaggcagaggtgggcagatcacctgaggtctggagttcgagaccagcctggccaacatggtgaaaccccgtctctactaaaaatattttaaaaaaagtattagctgggtgtggtgtgcacctgtagtcccagctactctggaggctgaggtaggagaatcgcttgaacccaggaggcagagagtgcagtgagtcaagattgcaccactgcactccagcttgggcaaccgagcgacactctgtctcaaagaaaataaataaataaaaataaagtttatctttaaggttttgtacatttttttcagtgtatgccttaggtaggttcttttttaatgttagtgtaacccagggacttctcttccattgcatcttctaagtaattacttatgaagtaccatatatgaaggctattgctgtttatatgttagtttttaccctgctcctttactaaattccaatcctttgaggtattggataaaaatatttttagcatttttcaaataacaggcagagtcaagggcttggtttcttttcttcccctcctgtcccctaccctccccttttttgagacagggtctcacttcttcgccgaggctggagtgcagtggtgcagttacggcttaccgcggcatctgcctccctggctgaaaagttcctcccacctcagcctcctgagtagctgggaccatagatgcacagcaccgcagctggctaatatttttgtattttttgtggaggcagtgtctccccatgttgcccagggtggtcccaaactcatgagctcaagcagtccgctcgccctggcctcctaaagtgtagggattataagcgtgagccactgcgcctggcctggggatcatgttttaacatgagaattagtggagacaaacacatgatatctaaataatagcaccatagtatacttgactagctttttaattattttttaaatatacaggaaggtaataagtaacaaagtaataatagtgaatagtttaagctcagttagcataatcgggcaaactttcatttgataaaagtgataagtagttttcagtggcttttttgtttaccagaaggaggtggtttttaaatacgtgcatccaagataaaatataaaaaaatgttcaggtttgctttcctacatagataaaataatatgtaactagctctcccaaatttcagcaacagttagtgaatgtttagccacaaatttgcagttaattatataatcagttcttaggattttatgaacaagttctatattctttgtgccttatacctagttgtaagcagtcattccacaattattttcctgaagtggcttggttaatgccacaccagaaacaggtcacagacaatagtgctgtaagaaatgtgtgaggaaagaggcacatgggaagtagctagctcgtgctggaggaactggaaaaaaacctcacatgggagatgacagttgagctgaattcttaactagagttgtaacagggcgaggcccttacatgcagaccacctgtgtggattaagataagacataaagtaatcttttaaaagaactattatttagaaacctggtatatgctacatggtgctgtgttatactgggtttgagaaagaatgggaagtgttacaaggattcagtggttggaaattaaggaagatagaaagttagtgttggatctgttttggctctttggtcatgcctttgtttttctcaaaatgaatgcagtgcccgtcccagaaaataccatatgagaagcgatttcataatgctgtgagagtctgttacagggacttgatcaagtctgagggccatgagagaaagtccctctgaggaagttgctttcaagctgacacctgaaggatgaagcagaattatcccagctgggatttgggaactggtgtttgaggctgaggactagcatgcatgataggaaaataacccagagtggcagaagtgggagtggtatgagatggcatcagagacgcagattcagggtcaaatcattcagagcctcctagaccatgtgaacacatgtattatgctgtggagatactgtttaataggcagtctgcttttttttctgcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcaggtaaggctggtaaggcctaactcttaatttttgtaccatataaaaaaacttttaatatggtaaagggattttcctttataatttttgcttttgtgtgatggtagggtagatagctaaggacttggggacccttttcaatatatattcgaaggttactgatgattgtaagaggttcagaggaaacagccaagaaagatttgagagtttacagctgtttctggaaatctggaaaccatggagttaaaaatcttaactaaagtctgcttggctctatttgcagtgttaatgtgctttctttattttttgtttgaacacagcatcctcatgtctatagtcctgtaatacagggtaatgctagaatgatggcaccaccaacacacgcccagcctggtttagtatcttcttcagcaactcagtacggggctcatgagcagacgcatgcgatgtatggtaggaagcactttgtttgtctcttccagtgtgtgtgactcttcttaatttaagtttctgaaaacatactctatctaagaataacctgaccttttatgacattgagggtcaagaatctgaaggaaaagatgaacccatttctttgcctgacttgctttataacttttggcaaatagtttctacttctgtacctggtcttcagatctctttcctgctttaactaaaatgtaatgatgtatataatggcaaagcatctttgtggagaaaggtacctttctcctcttcctcatcaatattatgctttggtatatcctgcctacgacatgcaagagaattttataataataaaagcataaaggtgttctccagcatgaaaacattttgcttcactacttgatctgagggtcactggcattacatattttttttgctgtttgttataatgataatactatgtttctacatcatgctgtattttaatggttgaatattatgtcatattagatatattttagacatgagtcacactttaaatataaccaatgtgaacagaatgctgaaatgaaaatgagaagtattttatgtaaaactaagcagtatttatatgtgagaataataagcaaaaaaacccatcttcgttttgtgactaaacagagaaatttgtgtagatcaacttagcagctgtctaaagtaccaaaataatagatttttcactgttgataatttaaaataaaatgtccatttgtatatcttatgatacagaattaatggattgcttcaaatgtttttcagaatatgtttttaaatagtactgatttcattaagatgttttgttctgaatatttctgagaactaccgtagtgtcgtttagttttcctatttgcgtttttggttgtttggagtaggggataattttggtttattcatacagttgaaaagtgtactgctatgagaatgagattatggttacatgtaactacatgggcatttcatttttaaagcctctttgaactttttgaaatactaagaatataaaatttttattttttaagtttagatgtcctgaacgagtatgtttaggcaaaattgagttatttaagaatttataggctgggcgcagtggctcacgcctgtaatcccagcactttgggaggccaaggctggcggatcatgaggtcaggagatcgagaccagcctggccaacatggtgaaaccccatctctactaaaaatccaaaaaattggccgggtgtggtggcatgtgcctgtagtcccggctacttcggaggctgaggcaacagaattgcttgaacccgggaggcagaggttgcagtgagccgagatcgcgccactacactctagcctgagcgacagagtgagactccatctccaaaaaaaaaaaaaaaaaaaaagaatttacagatttctggcaaaccttcttcttgagacattactacttttcataccacctctgtcctttttgaagaataaaagttttaacattccgtaggttaatgagaataggacttgggcagcagcaatcatccttcctgtcacctgtaacccacagcttatgctttcttcctggaggttcttgtctgccacaaaggctcactgctgataggaatttgtatatgatcaaaggtgtttagttttataaaacagttaagtccagtcttaattttccacattatcactttcaattttgtattgtggattacgcattttaaataaaaaattgtgtgattgctacattttggaaaacatttttttcaagaggcccatccgtaatttaattgtaaaagatactgacaaactaacttggtttattattttggttatgaccccgtcatttgacttgtctttagttgtcttaacggggactgaatatgcgtgcaaaggcacgattgatttatcatgctggcttttatgcaacttgtatatattttaacaattttcctgtttgctaaaggcttaggttaaaagttcattatgattgtttatacatttctggtgaatacatcatgatttaacaagtggaaagaacatctctttccttccattttctggcatactccccttggaatcagatctgaaacttttaagctaaaatttccattgcatttggagagtagttatttgtgtatgcatgcttttgagacattgtagcaataatactgtaatgttgagccgaatctttctcctcattgtgttcattcactgccaacatctggcttcatcttttggatgaatgttcattggttttgaaacagcctatagggtaaatactgtgtttgaggtacagatgattttcataactacttcctagaacatgtccatttgaagagcagtggggccttagaccccaaagtccatttatgtgtgggcaaataggaaatgttgcaaacaaaacaaagcactagatctaatgtccagtgaaatctggaatgaactagtcattagagccggttctttcatgccaggaaaaagttactcagccaaatctgaactactctcctgcagtttacacaggtggtatttaattgctgtctgtatggaggcaggctaggagcaaggctgtggacttgttgtgattgtcactagttaatcaagattccctttgtggtgcttaagaccctaaaaaggacactaggagctgggcatggtggctgacacctgtaatccaagaacttggggaggctgaagtggaggatcgcttagcccaggtgttcaagaccagtctaggcaagatggcgagatcccatctctaccaaaaaaaaaaaaaaaaaaaaaaaaaaaaagcccagtcatggtggcacatgcctgtagtcccacctacacaggaagctgagatgggaggatcacttgagtccaggactttgaggctacagtgagctatcatggcaccactgtaatccagcctgggtgacagagcaagaccctgtctctatttaaaaaaaagaaaacataagaaagaattgttttgttctatgccatcataagccataatttaatctgcttaagcatgttcttcattaaatctgcagtgatttatttgaattattagactttcaaagccttattatatcaaatataaacaaaatttgaagtacattcttataaactacaacaaacttacatagaagtgttaattttatactcatcttccctgaacaatttatattttataaatatattaaatatattgtaataaattttctcaaaggaaccaaatactttgagtatgaattgtgcttttctttttaagctacatcatatctaggtttttaaaacatttaatgcaaacagaagaacatgcacccagatgttggtgacaattttatgtcaccttttctcattcattaattgttatagccatagccaaaggcattgaaaacataggaccactaatgactgcaaaatgaaatcctgattattgtttttaaatttttagtatgtttaatacacatatgctaacattactgaacagttaaatgataaaataggataattattttattctaaaaaagtattgaccttgacctctttctagctatcttagaaagggcttttgtcaaaaaccttatctctttgatgtctctttttttgagatggagtctctccctgtcgcccaggctggagtgcagtggcgtgatctcagctcactgcacgctccgcctcctgcgttcacgccattctcctacctcagcctcccgagtagctaggactacaggcgcccgccaccatgcccggctaattttttgtattttgtttagtagagatggggtttcactgtgttagccaggatggtcttgatctcctgacctcgtgatccgcctgcctcagcctcccaaagtgctgggattacaggcgtgagccactgtgcccagcctctttttttttttttattttttatttattttttattttttttttaatttttgagaaggagtctccctctgccacccaggctggagtgcagtggcgcgatctcagctccctgcaaactccgcctcctgggttcaagcagttctcctgcctcagcctcctgagtagctgggactacaggtgcccgccaccacacctggctaatttttgtgtttttagtagagacagggtttcaccatgttggtcaggctggtcttgaattcccgacctcaggtgatccacccacctcagcctcccaaagtgctgggattacaggcgtgagccactgccccggcctctttgatgtctcttaatctaacttccatcattgcctctaccccatcccttctaagaagttactttaattttttttcctctcacatctactctttttttttttttttttttttttttgaggtagtctcactctgtcacccattctgaagtgcagcggtgcgatctcagctcactgcaacatctgcctcccaggttcaagcggtttttctgcctcagcctcccgagtaggtgggactacaggtgtgcgccaccacgaccggccaatttttgtatttttagtagagacggggtttcaccgtcttggccaggctgatctcgaacttctgaccttgtgatttgtctgcctaggcctcccaaagtgctgggattacagatgtgagccaccacgcccagcctcacatctactcttctaatccatctaattttgttttatggtgatgcttttacctttcagaaacagtaataatacaacttttccgactaactagagccattaggaagaattagatccagaatccttttttgatttgtttttggtagtttaatgcagataagtaagaaaatatagttaagttaaaaaaaaaaaaaatgaaaagcatccataatccctccacctgacaactgccttttaacattttgatgtgtatccttccaggtgtatttaaatacactcaaataccctacccctttatgtagacatgttttaataagaaataatattcatgtttatattcttgctatgatcctaaatttttggatccattactagataatctttcaggataatgacatttccattagtaatgtttttgcaaaattgtgtgtctattgaattaaacttgtaaaatagttttattttggtacatgatttatatcaaggttgttcagtagaatgccatgttggtgtttttattagataatgattttattccttttacttttaagcaagtcagcatgacaacttgacacctaagtacagaagaacagtgtcttccggtttagtcctttcttttaaaattctgtagcagtgtttaaagtgcttgtcatctcttatgaaaatgaattatgcatgaatacaaaaagaaattactaatatgtcaacctttccagaaaatttggaaaatgcacacctcaaaaggctaatttacctttctatttcccaaattcagcatgtcccaaattaccatacaacaaggagacaagcccttctttctactttgccagtgagttgggttttttatactaatttttaattgtacagtaaaacactttttaaaggatacatgttaagggagtagacttgttgaacaatattttccttgtgccagtcaaattattgaaagtacttatatatataaataattcagtttttaaaatggaaatacccaatttaagaaggctggagttaatgaaaaatggagttgtttcagaaatcaatttttgcataccaagcaaatgtgactgggaaatgcctaatattttccttgttagagaaacttcctaaacagctttatacacacacacacacacacacacacacacacacacaaacacacacacccaagccacaagcttggtataaatttaaaatgtttatttatacacacacacacacacacacacacacacacacacaccccaagccacaagcttggtataaacttaaaatgtttatttatattctgataagatgaaatttatgcctaccaggatttttaattgaataggattgatgaaatactaagggaaaaacttttcagtcctgtgcatggctaaaggtttaaaatactcaggaagggccaggcacggtggctcacacctgtaatcccagtgctttgggaggctgaggcgggtggatcatctgaggtcagcagttcaagaccagcctagccaacatggtaaaactccatctctactaaaaaatacaaaaatcagccatgcatgctggcatgcgcctataatctcagctactagggaggctgagacaggagaattgcttgaacttgggaggcagaggttgcagtgagccgaagtcgtgccactccactccagcctgggtggcagagcgaaattctgtctcaaaaaataaaatattcaggaagcagacccctcaggatatcttgagcttaagcaagagatcatgacctctcaggtcattatcttggacagcacaggtcccctctccccacctggcaaaaagtacagaaatagttgctccttcatggagaaagtctgggcagagctttcttctggaaatgaacttttaaggtacatttttcctatttgtagggcaatttgtaaaaataagggccggacgtggtggctcacgcctgtaatcccagtactttgggaggccgaggtgggtggattgcttgaggccaggagttcgagaacagcctggccaacatggtgaaaccctatctctaccaaagcatggtggcacgcacctgtagtcccagctacttgggaggcggaggcacaagagttccatgaaccctggaggtggaggttgcagtgagctgagattgtaccactgcactcaggcctgggcaacagagagagactctgtctcaaaataaaaaataaaaataaggctagtcttggactttggtatttaaataggaaggagtactaatatttgtagaaatcctttagaaatttgtgccattaatattgtcaccttgtatgaaatgttgtgttctagaggatattaaggattcaaattttatgttaggcacattttgagttattttggggtgactcaatgtctgactctactaaatgccatattagcatttaaaatgcatttgaccttaaatctttgttaattatgccatgacttggtatccaaaaataagctgatacatacatacatacatatatgtgtgtgtgtgtgtgtgtgtgtgtatatatatatatatgtatgtgtgtatatataatttatttggtgctaggaaatgttaaatttaatcctttaatagatgctctttaaaaaggagtcttgctgtatgtatatactattaaaggggaaactatgtctgtgattgtagtgtgtaaaagatagtaggtgattttattatgtactcaatttgaggtctcaaatgtagttatcctcaccatcttactgtctctgttagtagtttggtgttgttttcctggtaagtagctaaggtccttaatcattaacacctaagccttaattgccttagcacaacttcccctaaaagggagtatcagtactttttaaaagaaactaacagttgggctgctaatttaatctgctgcttcatttccccctgttctaagccattttatgatggtttggtcaagttgccttttattccccttttagagttttcaactttccttcacttccctttttctgaatttaacatcagatttacaagttggaagattttgttttgttttataagttttgcaatgctggtgatctcttatgacttgtgcatccaaagtcaaaatgacaaaacctagttacaaattaaacacacagctttctgtacttaatttgcttcagtgagatcacagctgaggaaactagttctggaatgtggttagtgttattaaggatttttgactgatcatatgtttagaatcttaaatatttatgtcaaggaacactgagtgggaaacttctggactaggtctggaccaaagaagcatatgtctttgattatctttaatctaaaagattttatgaagactaaagttttataaatagaagtttaactgatgaataaatcagtattacaaataaaattaactttatttttaacctctctgggatctttagccagaatgagcatatataacaaaagcagtgaaataatatgtgtgggtcagaacccactgcccttcccactccactctccttttccctgattctcctgtgttttttccttctttaccttatcttggttcctttttttttttttttcttttgagatggagtctcactctgtcgtccaggctggagtgcagtggtgcgatctcggctcactgcaacctccgcctcctaggttcaagcaattctctgcctcagcttccagagtagctgggattacaggcgcctgctgccacacccagctaattttttttgtatttttagtagagacagggtttcaccatcttggccaggctggtcttgaactcctgacctcgtgatcacctacctcggcccctggttccttttttgtctctcttgtcttccaagctatttttttccttggcttttaaattttcttcctaccctgctttgtgtcactgtcacttaactggcctatcaaggaaccgaactgtatttttgttactagtattgatttaaagtataagtttcacatttctcccaatttattattattatttatttatttatttgtttattttattttttgagacggagtttcgctcttgttgcccaagctggagtgcaatggtgtgatgtcggttcactgcaacctccacctcccgggttcaagctattctccttccccactctccctagtagctgtgattacaggtgcctgccaccacgcccagctaatttttgtatttttagtagagacagggtttcgccgtgttggccaagctggtctcgaactcctaacctcaggtgatccgcccgcctcggcctcccaaaatgctgggattacaagcgtgagccaccgtgcccggctccatttctcccaatttcaaattcaaggaggaaaagaattcctgattaaggtacttctttcagatcttttgagctagaacaaaaaaacaaagggaaatatttctaattaactctttttaaattttgtttacaacgtatgatacatattttacacatcctttgtggtttttgttcgtcttgtttttaatcaatgccttgcaagtttaccggtatttaggtagggaaaggattttgtttttgtttttttaaacaaagcctatgtacattcactcagcttgggtatttgtgctatgcatgcaaattagctatagattagaaaaccgtattatagtctttaaatactggtaaacttaaattgcagagatgccttttaaaaatgcatagtaaaaatatttcatctttacttttctcttcaaatgattttaagatttttacatttttccagttgatgaataacttaaattatgagatttcatgggcataattattttctatatttattgttactttttaatattcttaatactttgcttagaaggtatttaaaagtgaaatttcaaactttttagtacaaaatttcttgaataaataaagttacaaaaaaaaaacaaaaacctctgagattccgtactgtatctttatgaacctccatgaacagaatttgggatttgggaattgcttttccttagacagatttagattgttacaaatgacatttttaagaggctggggtggcggtaggggttagtgctaatggtttaacagtaggggaccatggacaactgtagacatcactatccagtagaacattttgtggctgggcgcggtggctcacgcctgtagtcccagcactttgggaggccaagacaagtggatcacctgaggtcaggagttcaagaccagccagaccaacatggtgaaaccctgtctctactaaaaatacaaaaaagttagccaggcgcgcctgtagtcctagctactcaggaggctgacacaggagaatcgcttgaacccgggaggcagaggttgcggtgagctgatatcacgccactgcactccaccctgggcaacagagcgagactccgtctcaaaacaacaacaaaactgcactgtccaccgtattagctacttagctacatgtggcttttttattattcaaaaataaatttttaggccgggtgcagttgctcacacctgtaatcccaacactttgggaggccgagatggacggatcacttgaggccaggagtttgagaccagcctggccaacatggtgaaaccccgtctctactaaaaatacaaaaattagccaggtaatcccagctactcagaggctgaagcaggagtatcactttaacccaggaggcggaggctgcagtgagccgagatcgctccactgcactccagcctgggtgacagcaagactgggtctcaaaaataaacaaacatggccgggcgcagtggctcatgcctgtaatcccagcactttgggaggccgaggcggatggatcacttgaggccagtagttcgagaccagcctggccaacatggtgaaacccgtctctactaaaaatacaaaaatcagccaggcatggtgatgcttgcctatagttccagctactcggcaggctgaggcaggagaatcgcttgaacccgggaggcggaggttgcagtgagccgagatggtgcccctgcactccagcctgggcaacagagcgagactctgtcaaaaattaaacaaataaatacatttttaaaatgaacgtaagatttttacaagtacaacaaactcaggttcgaaatttacatcaaatcttttagaccaagtcagtgcctatacaacttggaggagctggaagtaaacttaatgagtatgatgatgatggagggcctgttaataagccaccaagttagaaaaaaaggactgtcttatagacttatgggactgtgaagctcaggaaggcttcatcgtttgtacatcatttgttctagctcccagaagacgttcactactcttaaaaacattcagagactatgttgccacagttttcttgttaaaatattctggcatatgttaattcctacagtctggaaaattttcccagtgtataaacaaagctgctgtatccagtctaaactggatatgaaggaatattaatgccagctgtggcattggcagtggatgcacaggtgatcctagaactggctctttgccttgccctttcccctgctaagagatagctttgcagctggagacgtaactgttagggctggagagttggtggcccttagccctacaacacctaggattatagaactgctccatgtgcctagcctaaccctctgcacaccatttacgtggaatatacccagagccgtctatgctggtgactcggcagccttgcctaccagactgctggaactagggtgcctcttcccaaagctgtgcttgcttctctcaccaatcagtcctgcatatgtctgtgtttgctaacacgttatatgaagaatgtggggaactattttggaatcatttctgtgtatgggcttattatcttgagggattttaggatttgtttctcaagagagggctgggaactataccttgctagagttgtcttgagaacgctctattctcagctcattgcctcgtggaggttagttttttatcatcggtgtgctgtccatagtcactggaagcagtgaacacatcctactctgcttctgattctcaacttactgtttttgaagcacatgaacaggccaggcacggtggctcacgtctgtaatcccagcactttgggaggctgaagtgggcggatcatttgaggtcaggagtttgagatcagcctggccagcatggcgaaaccccatctctactaaaaatacaaaaattagctgggcgtggtggcacatgcctgtaatctcagctactcgggaggctgaggcaggagaattgcttgaacctgggaggcagaggttgcagtgagcctgggcaacagagtgagtgagacttatatctcaaaaaaaaacaaaaaacaaaaaactgaaagacatgaagaaatggtttttgtaccaaggtttggcccacgctgagattcacaaagaactggctttcagttcttatctttattttgatttaaactggcccatcatgttgtcctttgaagttagtctagtaaatttctttccaaagggctggggcactcagaagggagtttacttttctatatttatttcataaagcaaagatgggagatcctccattagggcttgggaaagtaaactgagtggcagaagggctcctgtgattagctgagagagactgtggtccttcggccctgatgatagatccctggccttgccacataccatacacagtgcccgcacccccatcccccaccacacccaatatagtctgtgccctcaggacattgctccagggcagtagcatggtgaggttagcctgatgatggccttgagctaaagagtgtgcacctaaaatgcacttgtttgagtagtttctgcctatgccttcaagttgcctttttgggaaaacctagtgaccgttaagagtaaatgcaaactaatttgattttaatatcatatgtagagctgtattatatgaaccaaatgctagtctgttaagcaatagctacacttattttttcaagacaatggatggtttaaatggagtcatctatagaaattggtagtggcgtgagttatgcattgtaaccatcaagaaagttcagttgatgaagtgtagaggagcgatggaggttgtcagacatcggttgtgtacatgctcctttttctttcactttagtttccacgggctcccttgctcagcagtatgcgcaccctaacgctaccctgcacccacatactccacaccctcagccttcagctacccccactggacagcagcaaagccaacatggtggaagtcatcctgcacccagtcctgttcaggtaagggcaactcagaggtctgcatggagtggcttctttatcctagtatctgagtgctttcttcaggtgccaggtatcgcatcgtcagaacacatggcatgtccaccctcgtgaagatggatacagctgtgcccctggggtggtggttttaagaatcacatttaaaggctgggcgcagtggctcacgcctgtaatcccaccactttgggaggccgaggcgggtggatcacgaggtcaggagattgagaccatcctggcgaacactgtgaaactccgtctctaataaaaatacaaaaaaattagccgggcgtggtggtgggcgcctgtagtcccagcttctcgggaggctgaggaaggagaatggcgtgaacccgggaggcggagcttgcagtgagcagagatcgcgccactgcactccagcttggacaacagcgagactctgtctcaaaaaaataaaaaattaaaaaaaatcacatttaagatacatgttgataataaggtgattggataagctctggaaacttgcagtaatgaaaaatcaaatttaacataaagttcataaggcaaattcctatttgcttgggactttttaatttctaaggtttatgtgatgaggttattttcctatgagcttcttgaattatgtttgctaatggaggcagttaaagatgtctttgatatctatcagttccctggggcagtagtcttttttgactttagtatgtatgctcagaagtttctaactgccagactgagaatcaggcttctgtaccctagaaaggagttgtccagatgggaggcacctccagccttgctcttaccaccctgtacattctcctgtactttccagtgaccctcatcataggcccaagtgtgcaaagcttagctttgtgggtatcccttggctgcttttcattaaagaagttttcctctcaattctttcctgtcgctttgcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggtaataccagctttagccaactttctgtgaaggccaagtagaatgtgaaggttatcagtaagcagctagaggctctcccagctaggaaaccctgtgtgtcatgccatttgcctgtctccctttccctctcaaatacacgtgatctggccctaagggaatgtttgtgtggttttgtcatgggatcagtgaaggtgctgattggtcagtcctttagttttccaactgagaccttaaaaatatctttgactctggaatgcaacccagtccttctttcctttctgtgtctgctttgctatgtctatatagcctcactactatatatatgtgtacatatatattcccctacacacttaccttggaagccaggcagggatgatggccttcacagagtctcagctctccgaagtgactaccggggcctgtcaacttgattgttactcacatgagttccagacacatctctccaattgttttccctggttatccatatatctgctttgaccataagttgtactcttgagagggcttggccttggacattggtgcagtgtaactagaagctggaagcacccaggtggtcccatttttctttaagagcagccctggaagcactttggagctcacctccagtgtaagctgctacaggtgaaaggtgtgcttgccatctcagtggttgctgtctgcatcagctgctgacaaaggtccctgcactccagggcccaggggattgtcttaatgaggagaaggagctgcactgaagttgggctctaacgctggccttgaggccctccctggggctgttacgggtgaattggctgtattagatgtctctgctactttcataacagaactctctgaggcgggtctaagtgagacctgccacaatgaattccatttcctgttaaatagtgcgccagtgaggctctggcaaggtgtgggctagagatgcgactcagttggatctatctctcagaaggctaccttgtaagtagagttccacagctctgggaagtttgggcgtcctcaccctgcaaagtttaggttctgtggtgtagcgcactgcagttgatttgctttttgatagtggggagggaagccggtttggtccgtgtgggccagcgtggtttggtggagtcagcttcataagagctggggtcctgtaggtgtctaccagaggctggtggctaagtaggcatgtgaacttacatgtaagtcagggatccctaaaacctcactctgtttttgtgctgaaagggcaaaaaggttaacacagggaagctcaaatttgccatgtgcccgtttgaatatgtgagagtaaaaacggcatttcatccaaggcttatcgtagtctagaacagtgcacagtgtgggaaaaaggaaacaagggctcttcctggccctgccaaccccctgcagagctggaatccagctgtttgggctgactaaaatcacctttccaacttgacagtgagtgagaccaggttgaacttggtacagagacgctgggctggcccagatgacttcaggttactcctttccatctcactggagccattaaaaactccaactcctcctcctcctcctgctccatcagcatatctctgagagagtcacgggggcctaagagtctcttttcactgcctggtgagcagaccagaagcagagggagagaggcaaatgaacagaggtccaagtaattcacatacttgactgtgacagtctctgcttattaatgtaatctgttttcctatttgaaagggatgttatctgcaaaactacctcaggccccacatggcagcctgattctgaagcatcattgaatcttgtatgatattaagttgagaaagctgcccttggatccagtgtctaatctttgtgaagatcttaccccatacatagaatacaatgatcagaaatgtcaagggttaggacagcacagccctgacttctacccaggctcacttgttgcctgctccctgacccttgcaggatctgcccaaaggtgaagcgcgtcttcaggtcaatagataatctactagagattgtccccagagaacagaactgggccctgaggcccaccgttgccctttcctgagagtcccagcccagtgaaaggaacacagttgacatgttgttgaagccggagatgttgcctgtatgcgtaaaagagctctctgtttcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcaggtgaggcgtgtgtgtgcaggggccgccggggcaccccaaagcattctgctcgcacaggtggaatggcaggcagggccagtgcttcaagccccgcatttgagaactagcaagacccgtccaggagtgtgcacaggagggactgtgacgatcagttcagcatcagggcctgaggcttccgggagccgagtctgtgtgtgttctgatggtatacaggatttggcttgatgagaagcagcagcagcagcaacagcagcctgatgcatgcctaggactcagttggccttccttgttatgacaggctggacagggcagtgttttccttcctgagtcccaaaagtctgacatgtggggggttattaccatggcagagtttgattgtagctctggagaagatactgctgagaaagcgctgtggatggactggctttgagtgtagcgttagccccagcccctgaacaggggagagcgccctgtgattgtgctctactacttgatggctgccatggcgatacttcacagtctgacctgttattctgaaagcaatactggtgcttggctaatatttggggagggggtttgttaaggcctttttttctaccccatgaacaagtcttctgggagttttatctgaagtggttttacgtctgactggtttgtttctacccacccacccaaccctccccactttggtgcagatgggagggggaaaagcgaattcaattttgagttttgttcagctagcacgaggatagtttacaatcatgtgctgcagagacactaggctgatgtgtggtgttgccagttttctgtttcaatgttcgcttttctttttacagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagcaaaagaagtaacaagagtgattcttgctgctattactgctaaaaaaaaaaaaaaaaaaaaatcaagacttggaacgcccttttactaaacttgacaaagtttcagtaaattcttaccgtcaaactgacggattattatttataaatcaagtttgatgaggtgatcactgtctacagtggttcaacttttaagttaagggaaaaacttttactttgtagataatataaaataaaaacttaaaaaaaatttaaaaaataaaaaaagttttaaaaactgatcaagttagtgtgtgtctgtataagctacttctttgtaggatacttaatatcaaagcaggtgtgctaagggtgcattttgaatatcccggaaggtagctgtgaaatgatttttcttcttcacccttagttctggttcaaggtatctctagaaaaagacaagactgagctattctctttggtggattagagatctgcttcaggaggaggaaggttggccagagttgggcagcactgaaattccacatcctcgggctgacaccgattctgtaagcttcctttttaatatctcctgaaccaaaatgagtgtcattagctggaagttcccaattcgggcatttttctactttaccagtagggggcaggagacactcagaaaaaaattgcaataaagaaatccagagggcatgaaggctgaaaagatacaaagatgtacaaagctgcttattgacatggatggactcataagcatttgttagtattcccagattgcaacggggaggacaaagggaagagcgagtatttgggcagggcaaggattttgtagagacaccatggtcttaatagagcctttaaatattatgacagcaaatcaagattctgaaaactttttaattcacatatagcaatttgtacattatagcaaaatttgcattattcaagaataagttacttgtacagtacataaaacaatacataaaaatttgccaaataccttctgcctataatgatacaagatgaatccactttatgttatcacaatgtgctgtatattctaaccaaacacaggatgtcagatgtgtccttgttaatatactcgcaagttcctctagcttgtgggagatgttagagctaacacatttgcagtaagggacttagtcctgaatagaaagcatgaaggaatctcaggcaaccctcagggaagagtccaaggccttgactttaggttaagaaactgttatgtaaaaatagtgttctctggcccaagattttaatgattgctattcctttttcctacggtccagaaatgatcaaaggcagaagatttataccagataaagccatatggattgctggtctaaaattcaaggcaggttagttgacttaattctttggtgctggtgactgttagtttgtaaaagttcaataagtcagatgaaggaagggatggtgccgggagctgtcaagctgtactggtggggtctgtaattagagctaactggagggatcatgatgtctactgtccagtttggtgttgagccatggctctcggtagaagttgccggctggggcctggtcaggactggaaggagagtggtgggatgtgctgtgcctatggtgggctagctgcagccagtggggtgcctgccccacactgctgcccaccccttcatcagctgattctgctcccacataaagaaaggtgttggcttagtgtcacttcttcctagagccatgggagttttctgtcagcatgtttttgagctgtcctggtaacttggacgggaagcagtctggaggtgggtgccttccaaatctctgccacagaa

SEQ ID NO: 3 - RNA, 863 nt, Homo sapiens
  source 1..863: mol_type = mRNA
gtctgtcggggctctctccccgccccctccggatcctgggnaagnacggnggacggggtggagacaagtgggccttggcccccgcacccctctgcgttcgtgtccgaggcggcggcgggggctcccgaactcccctgaaatcgtggggctccatgtggcctccggcagcgttccaccctcccccacctggggaagggaaggggtggggagtgcccggccccgtcccggccttcctccttcccccgccagacctctccggcgcgcgggtggtggccgatccgcattgctgttcgaggccgcagtggagaaggcgcctgtggaacatcgaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggaccctgcagcccctggatgcaggtgaactcacagccaatgagggaactggaggctttgnaaaatgacgtatctaatggatggaacccaaagatatgtttcgtttaatgaaaaaaattatggcgcaggggccaccgttgaaagcagtttatttcggatac

SEQ ID NO: 4 - skipped

SEQ ID NO: 5 - DNA, 27 bp, Synthetic construct
  source 1..27: mol_type = other DNA
accaaagagtagttaatggaggtgttc

SEQ ID NO: 6 - DNA, 19 bp, Synthetic construct
  source 1..19: mol_type = other DNA
agaaggtgggcgagaggaa

SEQ ID NO: 7 - DNA, 19 bp, Synthetic construct
  source 1..19: mol_type = other DNA
ctggccatcgccttgccca

SEQ ID NO: 8-10 - skipped (3 entries)

SEQ ID NO: 11 - DNA, 20 bp, Synthetic construct
  source 1..20: mol_type = other DNA
ccggctcgcacgccgggcgg

SEQ ID NO: 12 - DNA, 20 bp, Synthetic construct
  source 1..20: mol_type = other DNA
catacaccggctcgcacgcc

SEQ ID NO: 13 - DNA, 20 bp, Synthetic construct
  source 1..20: mol_type = other DNA
ggcttcagcgacatggtgag

SEQ ID NO: 14 - DNA, 20 bp, Synthetic construct
  source 1..20: mol_type = other DNA
cgacctctgcccaggccggg

SEQ ID NO: 15 - DNA, 20 bp, Synthetic construct
  source 1..20: mol_type = other DNA
tgcatagattccatcaaaag

SEQ ID NO: 16 - DNA, 20 bp, Synthetic construct
  source 1..20: mol_type = other DNA
aagtatatgaaccatcctca

SEQ ID NO: 17 - DNA, 20 bp, Synthetic construct
  source 1..20: mol_type = other DNA
ttcacttgtacttcacattt

SEQ ID NO: 18 - DNA, 20 bp, Synthetic construct
  source 1..20: mol_type = other DNA
tctgtacttttctcatgtgc

SEQ ID NO: 19 - DNA, 20 bp, Synthetic construct
  source 1..20: mol_type = other DNA
ctggattctgtacttttctc

SEQ ID NO: 20 - DNA, 20 bp, Synthetic construct
  source 1..20: mol_type = other DNA
ctctccattatttcttcacg

SEQ ID NO: 21 - DNA, 20 bp, Synthetic construct
  source 1..20: mol_type = other DNA
tctttaaactgtaccacaac

SEQ ID NO: 22 - DNA, 20 bp, Synthetic construct
  source 1..20: mol_type = other DNA
gagtcagtaaaagcatctct

SEQ ID NO: 23 - DNA, 20 bp, Synthetic construct
  source 1..20: mol_type = other DNA
cagggctccaggtccttctc

SEQ ID NO: 24 - DNA, 20 bp, Synthetic construct
  source 1..20: mol_type = other DNA
gcatcccagggctccaggtc

SEQ ID NO: 25 - DNA, 20 bp, Synthetic construct
  source 1..20: mol_type = other DNA
tcttcattatatcgaaacat

SEQ ID NO: 26 - DNA, 20 bp, Synthetic construct
  source 1..20: mol_type = other DNA
gctaactggtttgcccttgc

SEQ ID NO: 27 - DNA, 20 bp, Synthetic construct
  source 1..20: mol_type = other DNA
gtatttttcttcctcactcc

SEQ ID NO: 28 - DNA, 20 bp, Synthetic construct
  source 1..20: mol_type = other DNA
tgctgtgtatttttcttcct

SEQ ID NO: 29 - DNA, 20 bp, Synthetic construct
  source 1..20: mol_type = other DNA
gaaatctgaagtgtgagaag

SEQ ID NO: 30 - DNA, 20 bp, Synthetic construct
  source 1..20: mol_type = other DNA
cctccattaactactctttg

SEQ ID NO: 31 - DNA, 20 bp, Synthetic construct
  source 1..20: mol_type = other DNA
ggaacacctccattaactac

SEQ ID NO: 32 - DNA, 20 bp, Synthetic construct
  source 1..20: mol_type = other DNA
ggcgatggccagggaacacc

SEQ ID NO: 33 - DNA, 20 bp, Synthetic construct
  source 1..20: mol_type = other DNA
gtagcgagaaggtgggcgag

SEQ ID NO: 34 - DNA, 20 bp, Synthetic construct
  source 1..20: mol_type = other DNA
agagttgggacctgactggt

SEQ ID NO: 35 - DNA, 20 bp, Synthetic construct
  source 1..20: mol_type = other DNA
tggaagagagttgggacctg

SEQ ID NO: 36 - DNA, 20 bp, Synthetic construct
  source 1..20: mol_type = other DNA
ggagctggagaaccatgagc

SEQ ID NO: 37 - DNA, 20 bp, Synthetic construct
  source 1..20: mol_type = other DNA
gagacaggagctggagaacc

SEQ ID NO: 38 - DNA, 20 bp, Synthetic construct
  source 1..20: mol_type = other DNA
ttgtgggatacaaattctag

SEQ ID NO: 39 - DNA, 20 bp, Synthetic construct
  source 1..20: mol_type = other DNA
ggaaccccactgaccactga

SEQ ID NO: 40 - DNA, 20 bp, Synthetic construct
  source 1..20: mol_type = other DNA
tcttgaagcctggaatcttt

SEQ ID NO: 41 - DNA, 20 bp, Synthetic construct
  source 1..20: mol_type = other DNA
aacctaaaatcattcttaaa

SEQ ID NO: 42 - DNA, 20 bp, Synthetic construct
  source 1..20: mol_type = other DNA
agttgatccatagattcaga

SEQ ID NO: 43 - DNA, 20 bp, Synthetic construct
  source 1..20: mol_type = other DNA
ctggtacagttgctgctgct

SEQ ID NO: 44 - DNA, 20 bp, Synthetic construct
  source 1..20: mol_type = other DNA
ctgccactggtacagttgct

SEQ ID NO: 45 - DNA, 20 bp, Synthetic construct
  source 1..20: mol_type = other DNA
tttgcattgggattcaatgt

SEQ ID NO: 46 - DNA, 20 bp, Synthetic construct
  source 1..20: mol_type = other DNA
gaaggctttggctgagagaa

SEQ ID NO: 47 - DNA, 20 bp, Synthetic construct
  source 1..20: mol_type = other DNA
gtagtagaaggctttggctg

SEQ ID NO: 48 - DNA, 20 bp, Synthetic construct
  source 1..20: mol_type = other DNA
tgacccaccatagatgggct

SEQ ID NO: 49 - DNA, 20 bp, Synthetic construct
  source 1..20: mol_type = other DNA
ggtattgggtataaaggttg

SEQ ID NO: 50 - DNA, 20 bp, Synthetic construct
  source 1..20: mol_type = other DNA
gtcataggtattgggtataa

SEQ ID NO: 51 - DNA, 20 bp, Synthetic construct
  source 1..20: mol_type = other DNA
ggatgctgagactgataatg

SEQ ID NO: 52 - DNA, 20 bp, Synthetic construct
  source 1..20: mol_type = other DNA
acatgaggatgctgagactg

SEQ ID NO: 53 - DNA, 20 bp, Synthetic construct
  source 1..20: mol_type = other DNA
aatttgggacatgcatacat

SEQ ID NO: 54 - DNA, 20 bp, Synthetic construct
  source 1..20: mol_type = other DNA
gtctccttgttgtatggtaa

SEQ ID NO: 55 - DNA, 20 bp, Synthetic construct
  source 1..20: mol_type = other DNA
tgaacaggactgggtgcagg

SEQ ID NO: 56 - DNA, 20 bp, Synthetic construct
  source 1..20: mol_type = other DNA
gactgctgctgtggactggc

SEQ ID NO: 57 - DNA, 20 bp, Synthetic construct
  source 1..20: mol_type = other DNA
ctgactgtacatgagcctga

SEQ ID NO: 58 - DNA, 20 bp, Synthetic construct
  source 1..20: mol_type = other DNA
ccattcctgactgtacatga

SEQ ID NO: 59 - DNA, 20 bp, Synthetic construct
  source 1..20: mol_type = other DNA
cagttggatgagaaggaacc

SEQ ID NO: 60 - DNA, 20 bp, Synthetic construct
  source 1..20: mol_type = other DNA
catgggcagttggatgagaa

SEQ ID NO: 61 - DNA, 20 bp, Synthetic construct
  source 1..20: mol_type = other DNA
accgccgggtggctgtgtcg

SEQ ID NO: 62 - DNA, 20 bp, Synthetic construct
  source 1..20: mol_type = other DNA
tttgagcgagggcggcctgg

SEQ ID NO: 63 - DNA, 20 bp, Synthetic construct
  source 1..20: mol_type = other DNA
gctgtagtgcactttgagcg

SEQ ID NO: 64 - DNA, 20 bp, Synthetic construct
  source 1..20: mol_type = other DNA
agactggaatgggctgtagt

SEQ ID NO: 65 - DNA, 20 bp, Synthetic construct
  source 1..20: mol_type = other DNA
gcgctgttgtcgagactgga

SEQ ID NO: 66 - DNA, 20 bp, Synthetic construct
  source 1..20: mol_type = other DNA
ggaaatgcgctgttgtcgag

SEQ ID NO: 67 - DNA, 20 bp, Synthetic construct
  source 1..20: mol_type = other DNA
ggcttgtactgaagggtgcg

SEQ ID NO: 68 - DNA, 20 bp, Synthetic construct
  source 1..20: mol_type = other DNA
gtggtgggcttgtactgaag

SEQ ID NO: 69 - DNA, 20 bp, Synthetic construct
  source 1..20: mol_type = other DNA
ctgttggtggtgggcttgta

SEQ ID NO: 70 - DNA, 20 bp, Synthetic construct
  source 1..20: mol_type = other DNA
caactgctgttggtggtggg

SEQ ID NO: 71 - DNA, 20 bp, Synthetic construct
  source 1..20: mol_type = other DNA
gccttacaactgctgttggt

SEQ ID NO: 72 - DNA, 20 bp, Synthetic construct
  source 1..20: mol_type = other DNA
ttcggttcctccagggcagc

SEQ ID NO: 73 - DNA, 20 bp, Synthetic construct
  source 1..20: mol_type = other DNA
ttctagttttctgtgcttcc

SEQ ID NO: 74 - DNA, 20 bp, Synthetic construct
  source 1..20: mol_type = other DNA
aataaataacttccagtttc

SEQ ID NO: 75 - DNA, 20 bp, Synthetic construct
  source 1..20: mol_type = other DNA
gaatcactcttgttacttct

SEQ ID NO: 76 - DNA, 20 bp, Synthetic construct
  source 1..20: mol_type = other DNA
cagcaagaatcactcttgtt

SEQ ID NO: 77 - DNA, 20 bp, Synthetic construct
  source 1..20: mol_type = other DNA
tttataaataataatccgtc

SEQ ID NO: 78 - DNA, 20 bp, Synthetic construct
  source 1..20: mol_type = other DNA
aagttgaaccactgtagaca

SEQ ID NO: 79 - DNA, 20 bp, Synthetic construct
  source 1..20: mol_type = other DNA
atcggccaccacccgcgcgc

SEQ ID NO: 80 - DNA, 20 bp, Synthetic construct
  source 1..20: mol_type = other DNA
caaagggttaattaggatct

SEQ ID NO: 81 - DNA, 20 bp, Synthetic construct
  source 1..20: mol_type = other DNA
cccaaagggttaattaggat

SEQ ID NO: 82 - DNA, 20 bp, Synthetic construct
  source 1..20: mol_type = other DNA
aggacagtcatttgatttgt

SEQ ID NO: 83 - DNA, 20 bp, Synthetic construct
  source 1..20: mol_type = other DNA
ctttgaggacagtcatttga

SEQ ID NO: 84 - DNA, 20 bp, Synthetic construct
  source 1..20: mol_type = other DNA
ctgacagaacaaatgatatg

SEQ ID NO: 85 - DNA, 20 bp, Synthetic construct
  source 1..20: mol_type = other DNA
tattgggtataaaggcttga

SEQ ID NO: 86 - DNA, 20 bp, Synthetic construct
  source 1..20: mol_type = other DNA
ggtattgggtataaaggctt

SEQ ID NO: 87 - DNA, 20 bp, Synthetic construct
  source 1..20: mol_type = other DNA
ctcttttacgcatacaggca

SEQ ID NO: 88 - DNA, 20 bp, Synthetic construct
  source 1..20: mol_type = other DNA
aggaaggccaactgagtcct

SEQ ID NO: 89 - DNA, 20 bp, Synthetic construct
  source 1..20: mol_type = other DNA
ggtcagacggaagcagaacg

SEQ ID NO: 90 - DNA, 20 bp, Synthetic construct
  source 1..20: mol_type = other DNA
ccacctggctgcggcgaagc

SEQ ID NO: 91 - DNA, 20 bp, Synthetic construct
  source 1..20: mol_type = other DNA
gccgttgccgttgctaccaa

SEQ ID NO: 92 - DNA, 20 bp, Synthetic construct
  source 1..20: mol_type = other DNA
ggcccatacaccggctcgca

SEQ ID NO: 93 - DNA, 20 bp, Synthetic construct
  source 1..20: mol_type = other DNA
gcttcagcgacatggtgagg

SEQ ID NO: 94 - DNA, 20 bp, Synthetic construct
  source 1..20: mol_type = other DNA
ggacattggcagccgcgggc

SEQ ID NO: 95 - DNA, 20 bp, Synthetic construct
  source 1..20: mol_type = other DNA
gattccatcaaaagaaatcg

SEQ ID NO: 96 - DNA, 20 bp, Synthetic construct
  source 1..20: mol_type = other DNA
caactgatgtaagtatatga

SEQ ID NO: 97 - DNA, 20 bp, Synthetic construct
  source 1..20: mol_type = other DNA
ccaaatcacacttcggactg

SEQ ID NO: 98 - DNA, 20 bp, Synthetic construct
  source 1..20: mol_type = other DNA
ctcatgtgcggcatcaagta

SEQ ID NO: 99 - DNA, 20 bp, Synthetic construct
  source 1..20: mol_type = other DNA
catttgaacaaaatactctc

SEQ ID NO: 100 - DNA, 20 bp, Synthetic construct
  source 1..20: mol_type = other DNA
ctgatagcagagtcagtaaa

SEQ ID NO: 101 - DNA, 20 bp, Synthetic construct
  source 1..20: mol_type = other DNA
gggccactcgagctttgtac

SEQ ID NO: 102 - DNA, 20 bp, Synthetic construct
  source 1..20: mol_type = other DNA
aggaatatatttattttccc

SEQ ID NO: 103 - DNA, 20 bp, Synthetic construct
  source 1..20: mol_type = other DNA
cccatacgcggtgaattctg

SEQ ID NO: 104 - DNA, 20 bp, Synthetic construct
  source 1..20: mol_type = other DNA
tggagcccgatccaggctgg

SEQ ID NO: 105 - DNA, 20 bp, Synthetic construct
  source 1..20: mol_type = other DNA
agaagtggatcttgatggca

SEQ ID NO: 106 - DNA, 20 bp, Synthetic construct
  source 1..20: mol_type = other DNA
ggagaaccatgagcagaggg

SEQ ID NO: 107 - DNA, 20 bp, Synthetic construct
  source 1..20: mol_type = other DNA
ggcccttctgaagacatgcg

SEQ ID NO: 108 - DNA, 20 bp, Synthetic construct
  source 1..20: mol_type = other DNA
cactggatatggaacccctc

SEQ ID NO: 109 - DNA, 20 bp, Synthetic construct
  source 1..20: mol_type = other DNA
gtgggatacaaattctaggc

SEQ ID NO: 110 - DNA, 20 bp, Synthetic construct
  source 1..20: mol_type = other DNA
actgaccactgatgaccacg

SEQ ID NO: 111 - DNA, 20 bp, Synthetic construct
  source 1..20: mol_type = other DNA
ctgggtctatgagttttagg

SEQ ID NO: 112 - DNA, 20 bp, Synthetic construct
  source 1..20: mol_type = other DNA
tggaataataccagcttggg

SEQ ID NO: 113 - DNA, 20 bp, Synthetic construct
  source 1..20: mol_type = other DNA
ggcatggcaacagcttcagt

SEQ ID NO: 114 - DNA, 20 bp, Synthetic construct
  source 1..20: mol_type = other DNA
taggagatgcagctggaata

SEQ ID NO: 115 - DNA, 20 bp, Synthetic construct
  source 1..20: mol_type = other DNA
gaagcctggaatctttagcc

SEQ ID NO: 116 - DNA, 20 bp, Synthetic construct
  source 1..20: mol_type = other DNA
ccctgcaggagagttctgcc

SEQ ID NO: 117 - DNA, 20 bp, Synthetic construct
  source 1..20: mol_type = other DNA
ttcagaagtagaacttggct

SEQ ID NO: 118 - DNA, 20 bp, Synthetic construct
  source 1..20: mol_type = other DNA
caattttgtctttgatcaaa

SEQ ID NO: 119 - DNA, 20 bp, Synthetic construct
  source 1..20: mol_type = other DNA
tgttactaagtattgaaggg

SEQ ID NO: 120 - DNA, 20 bp, Synthetic construct
  source 1..20: mol_type = other DNA
aagtgacctcaggtcccctc

SEQ ID NO: 121 - DNA, 20 bp, Synthetic construct
  source 1..20: mol_type = other DNA
atgttgatttcctaacttgc

SEQ ID NO: 122 - DNA, 20 bp, Synthetic construct
  source 1..20: mol_type = other DNA
gtataaactggagttggctg

SEQ ID NO: 123 - DNA, 20 bp, Synthetic construct
  source 1..20: mol_type = other DNA
gtgcaaaacaaacaggctga

SEQ ID NO: 124 - DNA, 20 bp, Synthetic construct
  source 1..20: mol_type = other DNA
gactggatacatcatatttg

SEQ ID NO: 125 - DNA, 20 bp, Synthetic construct
  source 1..20: mol_type = other DNA
ggttgcacgcctgggctcac

SEQ ID NO: 126 - DNA, 20 bp, Synthetic construct
  source 1..20: mol_type = other DNA
tcataggtattgggtataaa

SEQ ID NO: 127 - DNA, 20 bp, Synthetic construct
  source 1..20: mol_type = other DNA
ttgattcactggcatgggcg

SEQ ID NO: 128 - DNA, 20 bp, Synthetic construct
  source 1..20: mol_type = other DNA
gatgatgctggtcttgccgc

SEQ ID NO: 129 - DNA, 20 bp, Synthetic construct
  source 1..20: mol_type = other DNA
atcattctagcattaccctg

SEQ ID NO: 130 - DNA, 20 bp, Synthetic construct
  source 1..20: mol_type = other DNA
atactaaaccaggctgggcg

SEQ ID NO: 131 - DNA, 20 bp, Synthetic construct
  source 1..20: mol_type = other DNA
acatgcatacatcgcatgcg

SEQ ID NO: 132 - DNA, 20 bp, Synthetic construct
  source 1..20: mol_type = other DNA
tagaaagaagggcttgtctc

SEQ ID NO: 133 - DNA, 20 bp, Synthetic construct
  source 1..20: mol_type = other DNA
cgcatactgctgagcaaggg

SEQ ID NO: 134 - DNA, 20 bp, Synthetic construct
  source 1..20: mol_type = other DNA
tagctgaaggctgagggtgt

SEQ ID NO: 135 - DNA, 20 bp, Synthetic construct
  source 1..20: mol_type = other DNA
caccatgttggctttgctgc

SEQ ID NO: 136 - DNA, 20 bp, Synthetic construct
  source 1..20: mol_type = other DNA
actgggtgcaggatgacttc

SEQ ID NO: 137 - DNA, 20 bp, Synthetic construct
  source 1..20: mol_type = other DNA
cgtggtaaatggctgactgc

SEQ ID NO: 138 - DNA, 20 bp, Synthetic construct
  source 1..20: mol_type = other DNA
ttggaggcaggtgtcatgga

SEQ ID NO: 139 - DNA, 20 bp, Synthetic construct
  source 1..20: mol_type = other DNA
tggcgcatgggcagttggat

SEQ ID NO: 140 - DNA, 20 bp, Synthetic construct
  source 1..20: mol_type = other DNA
ctttgagcgagggcggcctg

SEQ ID NO: 141 - DNA, 20 bp, Synthetic construct
  source 1..20: mol_type = other DNA
gtcgagactggaatgggctg

SEQ ID NO: 142 - DNA, 20 bp, Synthetic construct
  source 1..20: mol_type = other DNA
attcctattggatgttacaa

SEQ ID NO: 143 - DNA, 20 bp, Synthetic construct
  source 1..20: mol_type = other DNA
atcttccactgcaagtgaac

SEQ ID NO: 144 - DNA, 20 bp, Synthetic construct
  source 1..20: mol_type = other DNA
tatggaattatggaatagcc

SEQ ID NO: 145 - DNA, 20 bp, Synthetic construct
  source 1..20: mol_type = other DNA
gcaagaatcactcttgttac

SEQ ID NO: 146 - DNA, 20 bp, Synthetic construct
  source 1..20: mol_type = other DNA
tgtagacagtgatcacctca

SEQ ID NO: 147 - DNA, 20 bp, Synthetic construct
  source 1..20: mol_type = other DNA
ggccaaggcccacttgtctc

SEQ ID NO: 148 - DNA, 20 bp, Synthetic construct
  source 1..20: mol_type = other DNA
cactgcggcctcgaacagca

SEQ ID NO: 149 - DNA, 20 bp, Synthetic construct
  source 1..20: mol_type = other DNA
aaattcctcattttcttttc

SEQ ID NO: 150 - DNA, 20 bp, Synthetic construct
  source 1..20: mol_type = other DNA
gttatagtaatctgtaatca

SEQ ID NO: 151 - DNA, 20 bp, Synthetic construct
  source 1..20: mol_type = other DNA
aggattgtaaaatgatacag

SEQ ID NO: 152 - DNA, 20 bp, Synthetic construct
  source 1..20: mol_type = other DNA
gtaggattgtaaaatgatac

SEQ ID NO: 153 - DNA, 20 bp, Synthetic construct
  source 1..20: mol_type = other DNA
ttatatatgtaaattatatc

SEQ ID NO: 154 - DNA, 20 bp, Synthetic construct
  source 1..20: mol_type = other DNA
aaccactgatttatacactt

SEQ ID NO: 155 - DNA, 20 bp, Synthetic construct
  source 1..20: mol_type = other DNA
ttaaaaaccactgatttata

SEQ ID NO: 156 - DNA, 20 bp, Synthetic construct
  source 1..20: mol_type = other DNA
atatagcactctgctgtatt

SEQ ID NO: 157 - DNA, 20 bp, Synthetic construct
  source 1..20: mol_type = other DNA
taccaagcttgtggcttggg

SEQ ID NO: 158 - DNA, 20 bp, Synthetic construct
  source 1..20: mol_type = other DNA
ttataccaagcttgtggctt

SEQ ID NO: 159 - DNA, 20 bp, Synthetic construct
  source 1..20: mol_type = other DNA
cctcgatgttccacaggcgc

SEQ ID NO: 160 - DNA, 20 bp, Synthetic construct
  source 1..20: mol_type = other DNA
gagttcacctgcatccaggg

SEQ ID NO: 161 - DNA, 20 bp, Synthetic construct
  source 1..20: mol_type = other DNA
tccagttccctcattggctg

SEQ ID NO: 162 - DNA, 20 bp, Synthetic construct
  source 1..20: mol_type = other DNA
ggttccatccattagatacg

SEQ ID NO: 163 - DNA, 20 bp, Synthetic construct
  source 1..20: mol_type = other DNA
ttaaacgaaacatatctttg

SEQ ID NO: 164 - DNA, 20 bp, Synthetic construct
  source 1..20: mol_type = other DNA
gcccctgcgccataattttt

SEQ ID NO: 165 - DNA, 20 bp, Synthetic construct
  source 1..20: mol_type = other DNA
ataaactgctttcaacggtg